(19) United States
(12) Patent Application Publication
(10) Pub. No.: US 2006/0246005 A1
(43) Pub. Date: Nov. 2, 2006

(54) Poly(peptide) as a chelator: methods of manufacture and uses

(75) Inventors: David J. Yang, Sugar Land, TX (US); Dong-Fang Yu, Houston, TX (US); Chang Sok Oh, Houston, TX (US); Saady Kohanim, Sugar Land, TX (US); E. Edmund Kim, Houston, TX (US); Ali Azhdarinia, Houston, TX (US)

Correspondence Address: FULBRIGHT & JAWORSKI L.L.P., 600 CONGRESS AVE., SUITE 2400, AUSTIN, TX, 78701, US

(73) Assignee: THE BOARD OF REGENTS OF THE UNIVERSITY OF TEXAS SYSTEM

(21) Appl. No.: 11/394,664

(22) Filed: Mar. 31, 2006

Related U.S. Application Data
(60) Provisional application No. 60/667,815, filed on Apr. 1, 2005.

(51) Int. Cl. A61K 51/00

(52) U.S. Cl. 424/1.69; 424/178.1

(57) ABSTRACT

Novel compositions for imaging that include (a) a polypeptide that includes two or more consecutive amino acids that will function to non-covalently bind valent metal ions and (2) a valent metal ion chelated to at least one of the two consecutive amino acids, are disclosed. Also disclosed are methods of imaging using these novel compositions, such as methods of imaging a tumor within a subject. Methods of synthesizing an imaging agent and kits for preparing an imaging agent are also disclosed. Methods for determining the effectiveness of a candidate substance as an imaging agent that involve conjugating or chelating the candidate substance with a polypeptide that includes two or more consecutive amino acids that will function to non-covalently bind valent metal ions.

The present application is related to U.S. Provisional Patent Application 60/667,815, filed on Apr. 1, 2005, hereby incorporated by reference in its entirety.

### BACKGROUND OF THE INVENTION

1. Field of the Invention

The present invention relates generally to the fields of imaging, radiotherapy, labeling, chemotherapy, and chemical synthesis. More particularly, the invention concerns compositions of: (a) a polypeptide that includes two or more consecutive amino acids that will function to non-covalently bind valent metal ions, and (b) one or more valent metal ions non-covalently attached to at least one of the two consecutive amino acids. A second moiety, such as an imaging moiety, a therapeutic moiety, or a tissue targeting moiety, may be bound to the polypeptide. Other embodiments include compositions that includes (a) a polypeptide comprising within its sequence a tissue targeting amino acid sequence, a diagnostic amino acid sequence, and/or a therapeutic amino acid sequence, and (b) one or more valent metal ions non-covalently attached to the polypeptide. Methods of imaging using the aforementioned imaging agents, methods of synthesizing the aforementioned imaging agents, kits for preparing these imaging agents, and methods for determining the effectiveness of a candidate substance as an imaging agent that involve conjugating or chelating the candidate substance to a polypeptide that includes two or more consecutive amino acids that will function to bind valent metal ions are also disclosed. Methods of treating a hyperproliferative disease in a subject using the aforementioned compositions are also disclosed.

2. Description of Related Art

Biomedical imaging includes various modalities that are widely used by physicians and researchers to assist with not only the diagnosis of disease in a subject, but also to gain a greater understanding of normal structure and function of the body. Exemplary imaging modalities include PET, SPECT, gamma camera imaging, CT, MRI, ultrasound, and optical imaging.

In many instances, optimal imaging of a particular site within a subject requires the administration of a particular agent to the subject. Inorganic metals such as technetium (⁹⁹ mTc), iron, gadolinium, rhenium, manganese, cobolt, indium, platinum, copper, gallium or rhodium have proved to be a valuable component of many imaging agents.

Labeling molecules with inorganic metals can be achieved by chelating the metal to combinations of oxygen, sulfur and nitrogen atoms of particular compounds. Chelators such as sulfur colloid, diethylenetriaminepentaacetic acid (DTPA, O₄), ethylenediaminetetraacetic acid (EDTA, O₄), and DOTA (N₄) have been used for this purpose. However, inorganic metals that are chelated in this manner are of limited usefulness for imaging because of their fast clearance from the body.

In recent years, several amino acids have been labelled with either gamma radiation-emitting radionuclides (I-123, I-131) or positron-emitting radionuclides (C-11, N-13, F-18) (Layerman et al., 2002; Vaalburg et al., 1992). While it has been shown that some amino acids can be used to measure protein synthesis rate, others are used with the sole aim of measuring the rate of uptake of the agent into a cell (Layerman et al., 2002; Vaalburg et al., 1992).

The excitatory amino acid glutamate (Glu) is a potent neurotransmitter in the central nervous system and exerts its action via a variety of glutamate receptors (GluRs) (Chenu et al. 1998). Cyclotron-produced L-(N-13) glutamate has been used to visualize malignant intracranial tumors in patients (Reiman et al., 1982). PET (N-13)glutamate is rapidly taken up by a majority of brain tumors following the administration of L-(N-13)glutamate, and N-13 uptake is correlated with breakdown of the blood-brain barrier, as demonstrated by contrast CT or pertechnetate (Tc-99m) studies (Reiman et al., 1982).

L-(N-13)-glutamate has also been used to image an osteogenic sarcoma (Gelbard et al., 1979) and embryonal rhabdomyosarcoma (Sordillo et al., 1982). In these studies, serial quantitative measurements of the amount of N-13 taken up by the primary tumor showed a decrease of 40% after 10 wk of chemotherapy. The N-13 label appears to concentrate in the soft-tissue portion of the sarcoma, whereas ^{99m}Tc diphosphonate uptake was greatest in the regions where calcification was occurring (Gelbard et al., 1979; Sordillo et al., 1982).

One of the major limitations of [13N]amino acids is that their half life is considered to be too short for general clinical application. Further, no metabolic compartmental model has been investigated for [13N]amino acids. For routine application, reliable production of the radiopharmaceutical is essential, and no such reliable method for production has been identified for [13N]amino acids. For PET amino acid formulations, the main problems in production include complex multistep synthesis, low radiochemical yields, and complex purification methods. Thus, factors such as higher cost, availability and in some cases increased radiation exposure limit the clinical availability and usefulness of [13N]amino acids.

The preferred radioactive label for imaging agents is technetium (^{99m}Tc) due to its favorable half life (6 hrs), ease of production, wide availability, low energy (140 keV) and low cost. However, attaching ^{99m}Tc to drugs for imaging purposes is often a challenge. The longer half-life of isotopes such as ^{99m}Tc facilitates shipping of the radiolabelled amino acids to hospitals without an on-site cyclotron or dedicated radiochemistry laboratory.

Due to the success in tumor imaging with [13N] glutamate, there is the need for imaging agents and radiotherapeutic agents that can be produced more efficiently and less expensively than [13N] glutamate. There is also the need for agents that are more stable than [13N]glutamate, which can be efficiently and easily produced, and which can be made more readily available for clinical application, such as in the form of an kit. Furthermore, there is the need for imaging agents that are not rapidly cleared from the body so that the targeting potential of the imaging agent or radiotherapeutic agent with the site of interest can be prolonged to improve image quality.

¹⁸⁸Re has good characteristics for imaging and for potential therapeutic use because of its high β energy (2.1 MeV), short physical half-life (16.9 hr) and 155 keV gamma-ray emission for dosimetric and imaging purposes. The short physical half-life of ¹⁸⁸Re allows for higher doses compared with long-lived radionuclides. Furthermore, the short half-life reduces the problems of radioactive waste handing and storage. In particular, ¹⁸⁸Re is available from an in-house generator system similar to a ^{99m}Tc generator. ¹⁸⁸Re can be obtained from a ¹⁸⁸W/¹⁸⁸Re generator, which makes it very convenient for clinical use. Both ^{99m}Tc and ¹⁸⁸Re emit gamma rays, so the dosimetry generated based on ^{99m}Tc images is expected to be more accurate than that produced using the current standard radioisotope, Y-90.

Regarding imaging using positron emission tomography (PET), PET radiosynthesis must be rapid because the radioisotope will decay during lengthy chemical synthesis and higher risk of radiation exposure may occur during radiosynthesis. Cyclotron-based tracers are constrained by the availability of local cyclotron and its high cost. The Food and Drug Administration (FDA) permits radiopharmaceutical production in central commercial facilities under well-controlled conditions, and distributes these to local clinics where they are administered. Similarly, radionuclide generator systems that can be produced in a well-controlled facility are embraced by current FDA procedures and have a long history of successful clinical application. A generator uses a parent-daughter nuclide pair wherein a relatively long-lived parent isotope decays to a short-lived daughter isotope that is used for imaging. The parent isotope, which is produced at a cyclotron facility, can be shipped to a clinical site and from which the daughter isotope may be eluted on site for clinical use.

⁶⁸Ga has a high positron emitting quantity (89% of its total decay), therefore the main consideration is its spatial resolution, which depends on the positron range (energy), the non-colinearity of annihilating photons, intrinsic properties, size and geometry of the detector and the selection of the reconstruction algorithm. Aspects of the detector design, physical properties and their influence on system spatial resolution have been extensively addressed by many authors, leading to a continuous optimization of hardware. Although the maximum positron energy of ⁶⁸Ga (max=1.90 MeV, mean=0.89 MeV) is higher than that of ¹⁸F (max=0.63 MeV, mean=0.25 MeV), a study using Monte Carlo analysis on spatial resolution revealed that under the assumption of 3 mm spatial resolution of PET detectors, the conventional full width at half maximum (FWHM) of ¹⁸F and ⁶⁸Ga are indistinguishable in soft tissue (3.01 mm vs. 3.09 mm). It implies that with the spatial resolution at 5 to 7 mm of current clinical scanners, the imaging quality using ⁶⁸Ga-based tracers can be as good as that of ¹⁸F-based agents and have stimulated others to investigate potential ⁶⁸Ga-based imaging agents. Further, ⁶⁸Ga-based PET agents possess significant commercial potential because the isotope can be produced from a ⁶⁸Ge generator (275-day half-life) on site and serve as a convenient alternative to cyclotron-based PET isotopes, such as ¹⁸F or ¹³N.

### SUMMARY OF THE INVENTION

The present inventors have discovered certain novel imaging and radiotherapeutic agents that include a polypeptide that functions as a carrier as well as a chelator for a valent metal ion. Compared to DTPA-drug conjugates, these agents have a prolonged targeting potential with a site of interest in a subject. In certain embodiments, the polypeptide is a poly(glutamate) (GAP) or poly(aspartate) (AAP) peptide containing 5-60 acid moiety. In some embodiments, the polypeptides include four acid moieties that are reserved for ^{99m}Tc chelation. The present inventors have also discovered that it is possible to bind a second moiety to the polypeptide, such as a tissue targeting moiety, a therapeutic moiety, or an imaging moiety, such that the agent is suitable for multimodality imaging or radiochemotherapy. Such conjugation reactions could be conducted, for example, in aqueous (wet) or solvent (dry) conditions. The complexing of a metal ion to the polypeptide improves water solubility of the agent, and allows for use of the agent in contrast enhancement targeted imaging.

Certain embodiments of the present invention generally pertain to compositions of a polypeptide that includes within its sequence two or more consecutive amino acids that will function to non-covalently bind valent metal ions, and one or more valent metal ions non-covalently bound to at least one of the two consecutive amino acids. A “valent metal ion,” as discussed in greater detail in the specification below, includes any metal ion that is capable of forming a bond, such as a non-covalent bond, with another atom or a molecule. The other atom or molecule is often negatively charged.

Any amino acid, whether naturally occurring or synthetic, that can function to non-covalently bind a valent metal ion is contemplated for inclusion in the polypeptides of the present invention. Thus, the amino acid that can function to non-covalently bind a valent metal ion must be capable of being an electron donor. Such amino acids are discussed in greater detail in the specification below. For example, the amino acid may include a carboxyl moiety that can function to non-covalently bind a valent metal ion. In certain embodiments, the two or more consecutive amino acids that will function to non-covalently bind valent metal ions are selected from the group consisting of aspartate, glutamate, an analog of aspartate, an analog of glutamate, cysteine, lysine, arginine, glutamine, asparagine, glycine, ornithine, and a non-naturally occuring amino acid that includes two more more carboxyl groups.

In certain particular embodiments, the two or more consecutive amino acids are glutamate residues. In further embodiments, the two or more consecutive amino acids are aspartate residues. In other embodiments, the polypeptide includes both glutamate and aspartate residues in any ratio. In these embodiments, the polypeptide may comprise any number of consecutive glutamate and/or aspartate residues. For example, in some embodiments, the polypeptide includes at least 2 consecutive glutamate and/or aspartate residues. In further embodiments, the polypeptide includes at least 5 consecutive glutamate and/or aspartate residues. In more particular embodiments, the polypeptide includes at least 10 consecutive glutamate and/or aspartate residues. In still more particular embodiments, the polypeptide includes at least 20 consecutive glutamate and/or aspartate residues, In further embodiments, the polypeptide includes at least 50 consecutive glutamate and/or aspartate residues. The consecutive amino acid residues may be identical (e.g., all glutamate), or a combination of different types of amino acid residues (e.g., a mixture of glutamate and aspartate residues).

The polypeptide may be of any molecular weight. For example, in some embodiments the polypeptide has a molecular weight of 300 to 30,000 daltons. In general, it is contemplated that more particular embodiments of the present invention will be of a lower molecular weight, such as a molecular weight of 750 to 9,000 daltons. A molecular weight of 750 to 9,000 daltons contemplates a polypeptide of about 5 to about 60 consecutive amino acid residues. It is contemplated that the sequence of consecutive amino acids that will function to non-covalently bind valent metal ions set forth herein will be essentially pharmacologically inert, with minimal biological and/or pharmacological activity.

In certain embodiments of the present invention, the polypeptide is capable of chelating three to five valent metal ions through coordination to carboxyl moieties of glutamate, aspartate, an analog of glutamate, or an analog of aspartate. In these embodiments, the polypeptide may chelate any number of valent metal ions. For example, the polypeptide may chelate one to two hundred or more valent metal ions.

The valent metal ion may be any valent metal ion known to those of ordinary skill in the art to be capable non-covalently binding to an amino acid residue. For example, the valent metal ion may be a radionuclide. A radionuclide is an isotope of artificial or natural origin that exhibits radioactivity. In some embodiments, the valent metal ion may be selected from the group consisting of Tc-99m, Cu-60, Cu-61, Cu-62, Cu-67, In-111, Tl-201, Ga-67, Ga-68, As-72, Re-186, Re-188, Ho-166, Y-90, Sm-153, Sr-89, Gd-157, Bi-212, Bi-213, Fe-56, Mn-55, Lu-177, a valent iron ion, a valent manganese ion, a valent cobalt ion, a valent platinum ion, and a valent rhodium ion. In certain particular embodiments, the valent metal ion is Tc-99m, Re-188, or Ga-68.

In certain embodiments of the present invention, the polypeptide includes a second moiety bound to the polypeptide. The second moiety may be bound to the polypeptide in any manner known to those of ordinary skill in the art. For example, in some embodiment, the second moiety is bound in an amide or ester linkage to a carboxyl moiety of the polypeptide.

The second moiety may be any type of moiety. For example, in some embodiments, the second moiety is a tissue targeting moiety, a diagnostic moiety, or a therapeutic moiety. These moieties are discussed in greater detail in the specification below. In some embodiments, the the tissue-targeting moiety is a targeting ligand. For example, the targeting ligand may be a disease cell cycle targeting compound, an antimetabolite, a bioreductive agent, a signal transductive therapeutic agent, a cell cycle specific agent, a tumor angiogenesis targeting ligand, a tumor apoptosis targeting ligand, a disease receptor targeting ligand, a drug-based ligand, an antimicrobial, a tumor hypoxia targeting ligand, an agent that mimics glucose, amifostine, angiostatin, an EGF receptor ligand, monoclonal antibody C225, monoclonal antibody CD31, monoclonal antibody CD40, capecitabine, a COX-2 inhibitor, deoxycytidine, fullerene, herceptin, human serum albumin, lactose, leuteinizing hormone, pyridoxal, quinazoline, thalidomide, transferrin, or trimethyl lysine.

In certain particular embodiments of the present invention, the polypeptide includes 5 to 60 consecutive glutamate residues and a targeting ligand, wherein the targeting ligand is estradiol, galactose, lactose, cyclodextrin, colchicin, methotrexate, paclitaxel, doxorubicin, celebrex, metronidazole, adenosine, penciclovir, carnetin, estradiol (position 3), estradiol (position 17), linolenic acid, glucosamine, tetraacetate mannose, or folate, and wherein the valent metal ion is ^{99m}Tc.

In embodiments of the present invention wherein the polypeptide includes a diagnostic moiety, the diagnostic moiety may be an imaging moiety. Imaging moieties, discussed in greater detail below, may, in certain embodiments, be a contrast media. For example, the contrast media may be a CT contrast media, an MRI contrast media, an optical contrast media, and an ultrasound contrast media. Exemplary CT contrast media include iothalamate, iohexol, diatrizoate, iopamidol, ethiodol, and iopanoate. Exemplary MRI contrast media include gadolinium chelates (e.g., Gd-DOTA), manganese chelates (e.g., Mn-DPDP), chromium chelates (e.g., Cr-DEHIDA), and iron particles. Exemplary optical contrast media include fluorescein, a fluorescein derivative, indocyanine green, Oregon green, a derivative of Oregon green derivative, rhodamine green, a derivative of rhodamine green, an eosin, an erythrosin, Texas red, a derivative of Texas red, malachite green, nanogold sulfosuccinimidyl ester, cascade blue, a coumarin derivative, a naphthalene, a pyridyloxazole derivative, cascade yellow dye, and dapoxyl dye. Exemplary ultrasound contrast media include ultrasound perfluorinated contrast media, such as perfluorine or an analog of perfluorine.

In certain embodiments, the second moiety is a therapeutic moiety. Therapeutic moieties are discussed at length in the specification below. In some embodiments of the present invention, the therapeutic moiety is an anti-cancer moiety. Any anti-cancer agent known to those of ordinary skill in the art is contemplated for use as an anti-cancer moiety in the present invention, and the anti-cancer agent can be bound to the polypeptide of the present invention in any manner known to those of ordinary skill in the art, as addressed at length elsewhere in this specification. Exemplary anti-cancer moieties include a chelator capable of chelating to a therapeutic radiometallic substance, methotrexate, epipodophyllotoxin, vincristine, docetaxel, paclitaxel, daunomycin, doxorubicin, mitoxantrone, topotecan, bleomycin, gemcitabine, fludarabine, and 5-FUDR. In certain particular embodiments, the anti-cancer moiety is methotrexate.

In other embodiments, the anti-cancer moiety is a therapeutic radiometallic substance selected from the group consisting of Re-188, Re-186, Ho-166, Y-90, Sr-89, Sm-153. In further embodiments, the anti-cancer moiety is a substance capable of chelating to a therapeutic metal selected from the group consisting of arsenic, cobolt, copper, selenium, thallium and platinum.

The valent metal ion that is non-covalently attached to the polypeptide can be imaged by any method known to those of ordinary skill in the art. Exemplary methods of imaging are discussed at length in the specification below, and include PET and SPECT.

The present invention also generally pertains to compositions that include a polypeptide that includes within its sequence a tissue targeting amino acid sequence, a diagnostic amino acid sequence, and/or a therapeutic amino acid sequence; and one or more valent metal ions attached to one or more amino acid residues of the polypeptide. In certain of these embodiments, the polypeptide includes two or more consecutive glutamate residues. For example, in particular embodiments, the polypeptide comprises 5 to 60 consecutive glutamate residues. In other embodiments, the polypeptide includes two or more consecutive aspartate residues. For example, in some embodiments, the polypeptide includes 5 to 60 consecutive aspartate residues.

The tissue-targeting amino acid sequence may be a targeting ligand, such as a disease cell cycle targeting compound, an antimetabolite, a bioreductive agent, a signal transductive therapeutic agent, a cell cycle specific agent, a tumor angiogenesis targeting ligand, a tumor apoptosis targeting ligand, a disease receptor targeting ligand, a drug-based ligand, an antimicrobial, a tumor hypoxia targeting ligand, an agent that mimics glucose, amifostine, angiostatin, an EGF receptor ligand, monoclonal antibody C225, monoclonal antibody CD31, monoclonal antibody CD40, capecitabine, a COX-2 inhibitor, deoxycytidine, fullerene, herceptin, human serum albumin, lactose, leuteinizing hormone, pyridoxal, quinazoline, thalidomide, transferrin, or trimethyl lysine. The diagnostic amino acid sequence may be an imaging amino acid sequence, such as a CT contrast media, an MRI contrast media, and optical contrast media, or an ultrasound contrast media. As set forth above, the therapeutic amino acid sequence may be an anti-cancer amino acid sequence. In certain embodiments, the anti-cancer amino acid sequence is capable of chelating to a therapeutic metal selected from the group consisting of arsenic, cobolt, copper, selenium, thallium, or platinum.

The present invention also generally pertains to methods of synthesizing an imaging agent, that include: (1) obtaining a polypeptide comprising within its sequence two or more consecutive amino acids that will function to non-covalently bind valent metal ions; and (2) admixing said polypeptide with one or more valent metal ions and a reducing agent to obtain a valent metal ion-labeled polypeptide, wherein one or more valent metal ions non-covalently attaches to at least one of the two consecutive amino acids. The reducing agent can be any reducing agent known to those of ordinary skill in the art. For example, in certain embodiments, the reducing agent is a dithionite ion, a stannous ion, or a ferrous ion. In the methods of synthesis set forth herein, the polypeptide may be any of the polypeptides set forth above, the discussion of which is herein incorporated into this section.

In some embodiments of the present invention, the method of synthesizing an imaging agent is further defined as a method of synthesizing an agent for imaging and chemotherapy. In further embodiments of the present invention, the method of synthesizing an imaging agent is further defined as a method of synthesizing an agent for dual imaging. The imaging modalities used in these methods can be any imaging modality known to those of ordinary skill in the art. Exemplary methods, discussed at length in other parts of this specification, include PET, SPECT, MRI, CT, and optical imaging.

Further embodiments of the present invention generally pertain to methods of synthesizing an imaging agent, that involve: (1) obtaining a polypeptide that includes within its sequence a tissue targeting amino acid sequence, a diagnostic amino acid sequence, and/or a therapeutic amino acid sequence; and (2) admixing said polypeptide with one or more valent metal ions and a reducing agent to obtain a valent metal ion-labeled polypeptide. The reducing agent, as discussed above, can be any reducing agent known to those of ordinary skill in the art, such as dithionite ion, stannous ion, or ferrous ion. In certain embodiments, the polypeptide includes at least 2 consecutive glutamate residues or aspartate residues. In more particular embodiments, the polypeptide includes at least two consecutive glutamate or aspartate residues. Exemplary valent metal ions include any of those discussed above. In a particular embodiment, the valent metal ion is Tc-99m. The tissue-targeting amino acid sequence may be a tissue targeting ligand, such as any of those tissue-targeting ligands discussed above. Similarly, exemplary diagnostic amino acid sequences, imaging amino acid sequences, and therapeutic amino acid sequences include any of those sequences discussed above.

Further embodiments of the present invention generally pertain to methods of imaging a site within a subject that involve the steps of: (1) administering to the subject a diagnostically effective amount of any of the novel compositions of polypeptides and valent metal ions set forth above; and (2) detecting a signal from the valent metal ion-polypeptide chelate that is localized at the site. Any method known to those of ordinary skill in the art can be used to detect a signal from the valent metal iono-polypeptide chelate that is localized at the site. For example, a signal may be detected using PET, CT, SPECT, MRI, optical imaging, or ultrasound. In some embodiments, the method is further defined as a method of performing dual imaging and radiochemotherapy. Radiochemotherapy refers to therapy using a radiotherapeutic metallic substance, such as any of those substances set forth above. In further embodiments, the method of imaging is further defined as a method of performing dual imaging of a site within a subject. Any imaging modality known to those of ordinary skill in the art, including any of those methods discussed above, can be applied in the present invention.

Still further embodiments of the present invention generally pertain to kits for preparing an imaging agent, wherein the kit includes a sealed container that contains a predetermined quantity of a polypeptide that includes within its sequence two or more consecutive amino acids that will function to non-covalently bind valent metal ions; and a sufficient amount of a reducing agent to non-covalently bind a valent metal ion to at least one of the two consecutive amino acids. Any of the polypeptides set forth above that include two or more consecutive amino acids are contemplated for inclusion in these embodiments.

Additional embodiments of the present invention pertain to kits for preparing an imaging agent, wherein the kit includes a sealed container that contains a predetermined quantity of a polypeptide that includes within its sequence a tissue targeting amino acid sequence, a diagnostic amino acid sequence, and/or a therapeutic amino acid sequence; and a sufficient amount of a reducing agent to attach one or more valent metal ions to the polypeptide. Any of the polypeptides set forth above that include a tissue targeting amino acid sequence, a diagnostic amino acid sequence, and/or a therapeutic amino acid sequence, are contemplated for inclusion in the present invention. The polypeptide may include one or more such sequences, and may include any combination of such sequences. As set forth above, the polypeptide may include any number of consecutive amino acid residues. In certain embodiments, the polypeptide includes at least two consecutive glutamate or aspartate residues. In further embodiments, the polypeptide includes at least 5, at least 10, at least 20, or at least 50 consecutive glutamate or aspartate residues. In some particular embodiments, the polypeptide includes five to 60 consecutive aspartate or glutamate residuesresidues.

The present invention also generally pertains to methods of determining the effectiveness of a candidate substance as an imaging agent, wherein the method includes: (1) obtaining a candidate substance; (2) conjugating or chelating the candidate substance to a polypeptide that includes within its sequence two or more consecutive amino acids that will function to non-covalently bind valent metal ions; (3) introducing the candidate substance-polypeptide conjugate to a subject; and (4) detecting a signal from the candidate substance-polypeptide conjugate to determine the effectiveness of the candidate substance as an imaging agent. Any method known to those of ordinary skill in the art can be used to identify candidate substances. Exemplary methods are set forth in the specification below. Any method of conjugating or chelating the candidate substance to the polypeptide is contemplated by the present invention, and exemplary methods are set forth in the specification below. As discussed above, any method of detecting a signal from the candidate substance-polypeptide conjugate is contemplated by the present invention, and includes any of the methods for detecting a signal discussed above and elsewhere in this specification.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Synthetic scheme of GAP-3-EDL.

FIG. 2. 1H-NMR of GAP-EDL

FIG. 3. Cellular uptake in breast cancer cells (RBA CRL-1747, 4 μCi/50,0000 cells/well.

FIG. 4. Cellular uptake in human breast cancer cells (4 μCi/200,000/well) at 3 h:

FIG. 5. Cellular uptake in human ovarian cancer cells (4 μCi/50,000/well) at 3 h.

FIG. 6. 100,000 rat mammary tumor cells were incubated with ⁶⁸Ga-GAP-EDL at 30-240 min incubation. There was marked higher uptake in ⁶⁸Ga-GAP-EDL group compared to ⁶⁸Ga-GAP (*p<0.005, **p<0.0005).

FIG. 7. 100,000 rat mammary tumor cells were incubated with ⁶⁸Ga-GAP-EDL (0.1 mg/well) in the presence of unlabeled estrone. Cells were harvested at 90 min incubation. Results expressed as % uptake relative to control group. *p<0.005 compared to control group. There was a decreased uptake in the cells treated with estrone indicating the cellular uptake was via a ER-mediated process.

FIG. 8. ^{99m}Tc-GAP-EDL count density ratios in breast tumor-bearing rats. In vivo tumor (uterine)-to-tissue count density ratios of 99 mTc-GAP-EDL.

FIG. 9A-9B. A. Planar images of breast tumor-bearing rats after administration of ^{99m}Tc-GAP-EDL and ^{99m}Tc-DTPA showed that tumor could be visualized from 0.5-4 hours post-injection. B. A selected image at 55 min post-injection.

FIG. 10. Synthesis of GAP-EDL (position 17).

FIG. 11A-11B. A. Cellular uptake in breast cancer cells (RBA CRL-1747, 4 μCi/well). B. 30, 60, 120, and 180 min planar scintigraphy of Tc-99m-GAP and Tc-99m-GAP-Estradiol¹⁷ in breast tumor cell line bearing rats after 300 μCi/rat, i.v. injection, acquired 500,000 count to compare tumor to muscle visualization.

FIG. 12. Synthetic scheme of GAP-COXi.

FIG. 13. Proton NMR of GAP-COXi.

FIG. 14. Nuclear Imaging of ^{99m}Tc-GAP-COX-2 (COX2 inhibitor). Breast tumor-bearing rats were imaged with 99 mTc-GAP-COX-2 (300 uCi, i.v.) pre- and post-cisplatin treatment (4 mg/kg, i.v.). Selected planar images of ^{99m}Tc-GAP-COX-2 are presented at 0.5-2 hrs post-injection.

FIG. 15. Synthesis of GAP-DOX

FIG. 16. Cellular Uptake of ^{99m}Tc GAP agents. Cellular uptake in breast cancer cells (RBA CRL-1747, 4 μCi/50,000 cells/well FIG. 17. Synthesis of GAP-DG.

FIG. 18. Synthesis of GAP-GAL.

FIG. 19. Cellular uptake in breast cancer cells (RBA CRL-1747, 4 μCi/well)

FIG. 20. Uptake of ^{99m}Tc-GAP-DGAC in breast tumor bearing rats (n=3, 27.5 μCi/rat, i.v.).

FIG. 21. Tumor-to-tissues count density ratios of ^{99m}Tc-GAP-DGAC in breast tumor bearing rats.

FIG. 22. T/blood & b/muscle count density ratios of ^{99m}Tc-GAP-DGAC in breast tumor bearing rats.

FIG. 23. ^{99m}Tc-GAP-DGAC imaging in rabbits. Planar scinitgraphy of ^{99m}Tc-GAP-DGAC in VX2 tumor-bearing rabbits (1 mCi/rabbit, i.v. injection) demonstrated that tumor could be well-visualized. Tumor-to-nontumor ratios are shown. T=tumor.

FIG. 24. ^{99m}Tc-GAP-DGAC imaging in rabbits. Planar scinitgraphy of ^{99m}Tc-GAP-DGAC in VX2 tumor-bearing rabbits (1 mCi/rabbit, i.v. injection) demonstrated that tumor could be well-visualized. Tumor-to-nontumor ratios are shown. T=tumor.

FIG. 25. Synthetic scheme of GAP-LAS.

FIG. 26. Cellular uptake in human ovarian cancer cells (6 μCi/60,000/well) at 2 h.

FIG. 27. Cellular uptake in breast cancer cells (RBA CRL-1747, 3 μCi/50,000 cells/well) FIG. 28. Cellular uptake in human ovarian cancer cells (3 μCi/60,000/well) at 2 h.

FIG. 29. Cellular uptake in human cisplatin resistant ovarian cancer cells (2.4 [μCi/well).

FIG. 30. Tumoriblood ratio of compounds in breast tumor bearing rats (n=3/time interval, 20 μCi/rat, IV).

FIG. 31. Tumor to muscle ratio in breast tumor-bearing rats (n=3/time interval, 20 μCi/rat, IV).

FIG. 32. Synthesis of GAP-FOL.

FIG. 33. Synthesis of GAP-MN.

FIG. 34. Synthesis of GAP-MTX.

FIG. 35. 30, 60, 120, and 180 min imaging of ^{99m}Tc-GAP-TML in tumor bearing rats. 30, 60, 120, min planar scintigraphy of ^{99m}Tc-GAP-TLM in breast tumor cell line bearing rats after 300 μCi/rat, i.v. injection, acquired 500,000 count to demonstrate tumor to muscle and heart to muscle visualization.

FIG. 36. Tumor-to-muscle count density ratios of ^{99m}Tc-GAP, ^{99m}-Tc-GAP-adenosine, ^{99m}Tc-GAP-EDL¹⁷, and ^{99m}Tc-GAP-TML compounds in mammary tumor-bearing rats.

FIG. 37. Synthesis of GAP-ADN.

### DESCRIPTION OF ILLUSTRATIVE EMBODIMENTS

The present inventors have discovered certain novel imaging and radiotherapeutic agents that include a polypeptide as a carrier as well as a chelator for a metal complex. Exemplary polypeptide carriers include a poly(glutamate) (GAP) or poly(aspartate) (AAP) containing 5-60 amino acid residues. Glutamate (GAP) and aspartate (AAP) bind to glutamate/aspartate or folate receptors. The present inventors have also discovered that a second moiety such as a tissue targeting agent can be attached to the polypeptide. These imaging agents can be produced more efficiently and less expensively than agents such as [13N]glutamate, and are not as rapidly cleared from the body as [13N] so that the targeting potential of the agent with the site of interest in the body of a subject can be prolonged to improve image quality.

A. Polypeptides and Amino Acids

In certain embodiments, the present invention concerns novel compositions comprising (a) a polypeptide that includes within its sequence two or more consecutive amino acids that will function to non-covalently bind valent metal ions; and (b) one or more valent metal ions non-covalently attached to at least one of the two consecutive amino acids. In further embodiments, the present invention concerns (a) a polypeptide that includes within its sequence a tissue targeting amino acid sequence, a diagnostic amino acid sequence, and/or a therapeutic amino acid sequence; and (b) one or more valent metal attached to the polypeptide.

As used herein, a “polypeptide” refers to a consecutive series of two or more amino acids. The amino acids can be in L form, D form, or a racemic mixture of L and D form. In some embodiments, for example, the polypeptides of the present invention include a consecutive series of at least two amino acids. In further embodiments, the polypeptide includes a consecutive series of at least five amino acids. In further embodiments, the polypeptide includes a consecutive series of at least 10 amino acids. In still further embodiments, the polypeptide includes a consecutive series of at least 20 amino acids. In some particular embodiments, the polypeptide includes a consecutive series of 2-200 amino acids. In more particular embodiments, the polypeptide includes a consecutive series of 5 to 60 amino acids.

As discussed above, certain embodiments of the present invention include a polypeptide that includes within its sequence two or more consecutive amino acids that will function to non-covalently bind valent metal ion. Exemplary valent metal ions include Ga (+3), Re (+5), Tc-99m (+5), and Gd (+3). Any amino acid, whether naturally-occurring or non-naturally occurring, that is capable of binding a valent metal ion is contemplated for inclusion in the polypeptides of the present invention. Exemplary amino acids that are capable of binding valent metal ions include aspartate, glutamate, an analog of aspartate, an analog of glutamate, cysteine, lysine, arginine, glutamide, asparagine, glycine, ornithine, and any synthetic or non-naturally occurring amino acid that includes two or more carboxyl groups. For example, in some embodiments of the present invention, the polypeptide may include from two to about one thousand or more consecutive amino acid that are capable of binding valent metal ions.

As used herein, the term “glutamate” refers not only to glutamate, but also to glutamic acid. Included within this definition are salts of glutamate, such as the magnesium salt, calcium salt, potassium salt, zinc salt, and combinations thereof. The glutamate residue can be in either the D form or the L form.

As used herein, an “analog of glutamate” includes a glutamate residue that is radiolabeled at any position. For example, the aspartate may be radiolabeled with a positron-emitting radionuclide (e.g., C-11, N-13, F-18) or a gamma-emitting radionuclide (e.g., I-123, I-131). A radiolabeled glutamate residue can be produced by any method known to those of ordinary skill in the art, such as with a cyclotron (see, e.g., Reiman et al., 1982, pertaining to N-13-labeled L-glutamate, which is herein specifically incorporated by reference). Also included in the definition of “analog of glutamate” is a glutamate molecule wherein a hydrogen atom is replaced by a halogen atom, such as a fluorinated glutamate molecule (see, e.g., Layerman et al., 2002, which addresses fluorinated amino acids for tumor imaging with PET, herein specifically incorporated by reference).

As used herein, the term “aspartate” refers not only to aspartate, but also to aspartic acid. The aspartate residue can be in either the D form or the L form. Included within this definition are salts of aspartate, including the magnesium salt, calcium salt, potassium salt, zinc salt, and combinations thereof.

As used herein, an “analog of aspartate” includes an aspartate residue that is radiolabeled at any position. For example, the aspartate may be radiolabeled with a positron-emitting radionuclide (e.g., C-11, N-13, F-18) or a gamma-emitting radionuclide (e.g., I-123, I-131). A radiolabeled aspartate residue can be produced by any method known to those of ordinary skill in the art, such as with a cyclotron. Also included in the definition of “analog of aspartate” is an aspartate molecule wherein a hydrogen atom is replaced by a halogen atom, such as a fluorinated aspartate molecule (see, e.g., Layerman et al., 2002, herein specifically incorporated by reference).

A non-naturally occurring amino acid that includes two or more carboxyl groups is defined herein to refer to an amino acid of the following chemical structure: 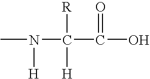
wherein R is any moiety that includes one or more carboxyl groups. For example, R may be an alkyl group, an alkenyl group, an alkynyl group, an alkoxy group an aryl group, an alkylaryl group, a carbocyclic aryl group, a heterocyclic aryl group, an amide group, a thioamide group, an ester group, an amine group, a thioether group, a sulfonyl group, or any other group known to those of skill in the art, as long as the group includes one or more carboxyl substituents. In addition to the one or more carboxyl groups, the R group may include additional substituents, such as one or more hydroxyl, cyano, alkoxy, halogen, ═O, ═S, NO₂, N(CH₃)₂, amino, or SH groups.

An “alkyl” group refers to a saturated aliphatic hydrocarbon, including straight-chain, branched chain, and cyclic alkyl groups. Preferably, the alkyl group has 1 to 12 carbons.

An “alkenyl” group refers to an unsaturated hydrocarbon group containing at least one carbon-carbon double bond, including straight-chain, branched-chain, and cyclic groups. Preferably, the alkenyl group has 1 to 12 carbons.

An “alkynyl” group refers to an unsaturated hydrocarbon group containing at least one carbon-carbon triple bond, including straight-chain, branched chain, and cyclic groups. Preferably, the alkynyl group has 1 to 12 carbons. More perferably it is a lower alkynyl of from 1 to 7 carbons, more preferably 1 to 4 carbons.

An “alkoxy” group refers to an “—O-alkyl” group, where “alkyl” is defined above.

An “aryl” group refers to an aromatic group which has at least one ring having a conjugated pi electron system, and includes carbocyclic aryl, heterocyclic aryl, and biaryl groups, all of which may be optionally substituted. Preferrably, the aryl is a substituted or unsubstituted phenyl or pyridyl. Preferred aryl substituent(s) are halogen, trihalomethyl, hydroxyl, SH, OH, NO₂, amine, an ester (e.g., COOH), thioether, cyano, alkoxy, alkyl, and amino groups.

An “alkylaryl” group refers to an alkyl (as described above), covalently joined to an aryl group (as described above). Preferably, the alkyl is a lower alkyl.

“Carbocyclic aryl” groups are groups wherein the ring atoms on the aromatic ring are all carbon atoms. The carbon atoms are optionally substituted with preferred groups as described for aryl groups above.

“Heterocyclic aryl” groups are groups having from 1 to 3 heteroatoms as ring atoms in the aromatic ring and the remainder of the ring atoms are carbon atoms. Siutable heteroatoms include oxygen, sulfur, and nitrogen, and include furanyl, thienyl, pyridyl, pyrrolyl, N-lower alkyl pyrrolo, pyrimidyl, pyrazinyl, imidazoyl, and the like, all optionally substituted.

An “amide” refers to a —C(O)—NH—R¹, where R¹ is either alkyl, aryl, alkylaryl, or hydrogen.

A “thioamide” refers to a —C(S)—NH—R¹, where R¹ is either alkyl, aryl, alkylaryl, or hydrogen.

An “ester” refers to a —C(O)—OR′, where R′ is either alkyl, aryl, alkylaryl, or hydrogen.

An “amine” refers to a —N(R″)R′″, where R″ and R′″ is each independently either hydrogen, alkyl, aryl, or alkylaryl, provided that R″ and R′″ are not both hydrogen.

A “thioether” refers to —S—R², where R² is either alkyl, aryl, or alkylaryl.

A “sulfonyl” refers to —S(O)₂—R³, where R³ is aryl, C(CN)═C-aryl, CH₂—CN, alkylaryl, NH-alkyl, NH-alkylaryl, or NH-aryl.

In addition, the polypeptides of the compositions of the present invention may include any number of amino acids other than amino acids that function to non-covalently bind valent metal ions. These additional amino acids may be at either the C-terminal end or N-terminal end of a sequence of two or more amino acids that can function to bind valent metal ions. Alternatively, the additional amino acids may be interposed within a sequence of consecutive amino acids that can function to bind valent metal ions. Furthermore, in some embodiments, the polypeptides may be branched. Thus, the polypeptides of the compositions of the present invention may include a total of 2 to about 1000 or more total amino acid residues, so long as it includes at least two consecutive amino that will function to bind valent metal ions.

In certain embodiments, the amino acid residues of the polypeptide are sequential, without any non-amino molecule interrupting the sequence of amino molecule residues. In other embodiments, the polypeptide may include one or more non-amino molecule moieties.

As used herein, an “amino acid” refers to any amino acid, amino acid derivative or amino acid mimic as would be known to one of ordinary skill in the art. The amino acid can be in L form or D form. Accordingly, the term “amino acid” encompasses amino molecule sequences comprising at least one of the 20 common amino acids in naturally synthesized proteins, any of the analogs of aspartate or glutamate set forth above, any non-naturally occurring amino acid that includes two or more carboxyl groups as set forth above, or any other modified or unusual amino acid, including but not limited to those shown on Table 1 below.

TABLE 1
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino-propionic acid | AHyl | allo-Hydroxylysine |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In certain embodiments, the polypeptide-containing compositions of the present invention comprises a biocompatible protein, polypeptide or peptide. As used herein, the term “biocompatible” refers to a substance which produces no significant untoward effects when applied to, or administered to, a given subject according to the methods and amounts described herein. Subjects include, but are not limited to, mammals such as laboratory animals (e.g., rats, mice, rabbits), and humans. Such untoward or undesirable effects are those such as significant toxicity or adverse immunological reactions. In certain embodiments, the polypeptide-containing compositions will be synthetic polypeptides that are essentially free from toxins, pathogens and harmful immunogens.

The polypeptides included in the compositions of the present invention may be made by any technique known to those of skill in the art, including standard molecular biological techniques, isolation from natural sources, or chemical synthesis.

In certain embodiments, the polypeptide may be purified. Generally, “purified” will refer to a specific polypeptide composition that has been subjected to fractionation to remove various other amino acid sequences, and which composition substantially retains its activity, as may be assessed, for example, by the protein assays, as would be known to one of ordinary skill in the art.

B. Valent Metal Ions

As set forth above, certain embodiments of the present invention pertain to compositions that include a polypeptides that includes within its sequence two or more consecutive amino acids that will function to non-covalently bind valent metal ions. A “valent metal ion” is defined herein to refer to a metal ion that is capable of forming a bond, such as a non-covalent bond, with another atom or a molecule. The other atom or molecule may be negatively charged. Any valent metal ion known to those of ordinary skill in the art is contemplated for inclusion in the compositions of the present invention. One of ordinary skill in the art would be familiar with the valent metal ions and their application. In certain particular embodiments of the compositions of the present invention, the valent metal ion is a radionuclide. Examples of valent metal ions to be employed in the compositions of the present invention include Tc-99m, Cu-60, Cu-61, Cu-62, Cu-67, In-111, Tl-201, Ga-67, Ga-68, As-72, Re-186, Re-188, Ho-166, Y-90, Sm-153, Sr-89, Gd-157, Bi-212, Bi-213

Due to better imaging characteristics and lower price, attempts have been made to replace the ¹²³I, ¹³¹I, ⁶⁷Ga and ¹¹¹In labeled compounds with corresponding ^{99m}Tc labeled compounds when possible. Due to favorable physical characteristics as well as extremely low price ($0.21 /mCi), ^{99m}Tc has been preferred to label radiopharmaceuticals.

A number of factors must be considered for optimal radioimaging in humans. To maximize the efficiency of detection, a valent metal ion that emits gamma energy in the 100 to 200 keV range is preferred. A “gamma emitter” is herein defined as an agent that emits gamma energy of any range. One of ordinary skill in the art would be familiar with the various valent metal ions that are gamma emitters. To minimize the absorbed radiation dose to the patient, the physical half-life of the radionuclide should be as short as the imaging procedure will allow. To allow for examinations to be performed on any day and at any time of the day, it is advantageous to have a source of the radionuclide always available at the clinical site. ^{99m}Tc is a preferred radionuclide because it emits gamma radiation at 140 keV, it has a physical half-life of 6 hours, and it is readily available on-site using a molybdenum-99/technetium-99m generator. One of ordinary skill in the art would be familiar with methods to determine optimal radioimaging in humans.

The polypeptides of the present invention may include one or more valent metal ions chelated to the polypeptide. The chelation, in particular embodiments, is to a carboxyl moiety of glutamate, aspartate, the analog of glutamate, or the analog of aspartate. In some embodiments, chelation of the valent metal ion is to a second moiety, such as to carboxyl groups of a second moiety. In one embodiment, chelation of the valent metal ion is to a carboxyl group of a glutamate, aspartate, analog of glutamate, or analog of aspartate of the polypeptide, and to one or more carboxyl groups of a second moiety. In further embodiments, the valent metal ion is chelated to three or more glutamate carboxyl moieties of the polypeptide. In other embodiments, the valent metal ion is chelated to three or more aspartate moieties of the polypeptide. These embodiments may include multiple valent metal ions chelated to poly(glutamate) or poly(aspartate) polypeptide.

In certain particular embodiments of the present invention, the valent metal ion is a therapeutic valent metal ion. For example, in some embodiments, the valent metal ion is a therapeutic radionuclide that is a beta-emitter. As herein defined, a beta emitter is any agent that emits beta energy of any range. Examples of beta emitters include Re-188, Re-186, Ho-166, Y-90, Bi-212, Bi-213, and Sn-153. The beta emitters may or may not also be gamma emitters. One of ordinary skill in the art would be familiar with the use of beta emitters in the treatment of hyperproliferative disease, such as cancer.

In further embodiments of the compositions of the present invention, the valent metal ion is a therapeutic valent metal ion that is not a beta emitter or a gamma emitter. For example, the therapeutic metal ion may be platinum, cobalt, copper, arsenic, selenium or thallium. Compositions including these therapeutic metal ions may be applied in methods directed to the treatment of hyperproliferative disease, such as the treatment of cancer. Methods of performing dual chemotherapy and radiation therapy that involve the compositions of the present invention are discussed in greater detail below.

C. Therapeutic Moieties

In certain embodiments of the compositions of the present invention, a second moiety is bound to the polypeptide. A “moiety” is defined herein to be a part of a molecule. In certain particular embodiments, the second moiety is a therapeutic moiety. A “therapeutic moiety” is defined herein to refer to any therapeutic agent. A “therapeutic agent” is defined herein to include any compound or substance or drug that can be administered to a subject, or contacted with a cell or tissue, for the purpose of treating a disease or disorder, or preventing a disease or disorder, or treating or preventing an alteration or disruption of a normal physiologic process. For example, the therapeutic moiety may be an anti-cancer moiety, such as a chemotherapeutic agent. In certain embodiments of the present invention, the therapeutic moiety is a therapeutic amino acid sequence that is fused or chemically conjugated to the therapeutic amino acid sequence. Such chemical conjugates and fusion proteins are discussed further in other parts of this specification.

Examples of anti-cancer moieties include any chemotherapeutic agent known to those of ordinary skill in the art. Examples of such chemotherapeutic agents include, but are not limited to, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing. In certain particular embodiments, the anti-cancer moiety is methotrexate.

Additional examples of anticancer agents include those drugs of choice for cancer chemotherapy listed in Table 2:

TABLE 2
DRUGS OF CHOICE FOR CANCER CHEMOTHERAPY
The tables that follow list drugs used for treatment of cancer in the USA and
Canada and their major adverse effects. The Drugs of Choice listing based on the
opinions of Medical Letter consultants. Some drugs are listed for indications for which
they have not been approved by the US Food and Drug Administration. Anticancer drugs
and their adverse effects follow. For purposes of the present invention, these lists are
meant to be exemplary and not exhaustive.
DRUGS OF CHOICE

| Cancer | Drugs of Choice | Some alternatives |
|---|---|---|
| Adrenocortical** | Mitotane | Doxorubicin, streptozocin, |
| | Cisplatin | etoposide |
| Bladder* | Local: Instillation of BCG | Instillation of mitomycin, |
| | Systemic: Methotrexate + vinblastine + doxorubicin + claplatin | doxorubicin or thiotape |
| | (MVAC) | Pecitaxel, substitution of |
| | Claplatin + Methotrexate + vinblastine | carboplatin for claplatin in |
| | (CMV) | combinations |
| Brain | | |
| Anaplastic astrocytoma* | Procarbazine + lamuatine + vincristine | Carmustine, Claplatin |
| Anaplastic oligodendro- | Procarbazine + lamustine + vincristine | Carmustine, Claplatin |
| Giloma* | | |
| Gilabiastome** | Carmustine or lamustine | Procarbazine, claplatin |
| Medulloblastoma | Vincristine + carmustine ± mechiorethamine ± methotrexate | Etoposide |
| | Mechiorethamine + vincristine + procarbazine + prednisone | |
| | (MOPP) | |
| | Vincristine + claplatin ± cyclophosphamide | |
| Primary central nervous | Methotrexate (high dose Intravenous and/or | |
| system lymphoma | Intrathecal) ± cytarabine (Intravenous and/or | |
| | Intrathecal) | |
| | Cyclophosphamide + Doxorubicin + vincristine + prednisone | |
| | (CHOP) | |
| Breast | Adjuvant¹: Cyclophosphamide + methotrexate + fluorouracil | |
| | (CMF); | |
| | Cyclophosphamide + Doxorubicin ± fluorouracil | |
| | (AC or CAF); Tamoxifen | |
| | Metastatic: Cyclophosphamide + methotrexate + | Paclitaxel; thiotepa + |
| | fluorouracil | Doxorubicin + vin-blastine; |
| | (CMF) or | mitomycin + vinblastine; |
| | Cyclophosphamide + duxorubicin ± | mitomycin + methotrexate + |
| | fluorouracil | mitoxantrone; |
| | (AC or CAF) for receptor- | fluorouracil by |
| | negative and/or hormone-refractory; | continuous infusion; Bone |
| | Tamoxifen for receptor-positive and/or | marrow transplant³ |
| | hormone-sensitive² | |
| Cervix** | Claplatin | Chlorambucil, vincristine, |
| | Ifosfamide with means | fluorouracil, Doxorubicin, |
| | Bleomycin + ifosfamide with means + claplatin | methotrexate, altretamine |
| Chorlocarcinoma | Methotrexate ± leucovorin | Methotrexate + dactinomycin + |
| | Dactinomycin | cyclophosphamide (MAC) |
| | | Etoposide + methotrexate + |
| | | dactinomycin + |
| | | cyclophosphamide + vincristine |
| Colorectal* | Adjuvant colon⁴: Fluorouracil + levam-isole; | Hepatic metastases: |
| | fluorouracil + leucovorin | Intrahepatic-arterial floxuridine |
| | Metastatic: fluorouracil + leucovorin | Mitomycin |
| Embryonal rhabdomyosar-coma⁵ | Vincristine + dectinomycin ± cyclophasphamide | Same + Doxorubicin |
| | Vincristine + ifosfamide with means + etoposide | |
| Endometrial** | Megastrol or another progestin | fluorouracil, tamoxifen, |
| | Doxorubicin + claplatin ± cyclophosphamide | altretamine |
| Esophageal* | Claplatin + fluorouracil | Doxorubicin, methotraxate, |
| | | mitomycin |
| Ewing's sarcoma⁵ | Cyclophosphamide (or ifosfamide with | CAV + etoposide |
| | means) + Doxorubicin + vincristine (CAV) ± dactinomycin | |
| Gastric** | Fluorouracil ± leucavorin | Claplatin Doxorubicin, |
| | | etoposide, methotrexate + leucovorin, |
| | | mitomycin |
| Head and neck squambus cell*⁶ | Claplatin + fluorouracil | Blomycin, carboplatin, paclitaxel |
| | Methotrexate | |
| Islet cell** | Streptozocin + Doxorubicin | Streptozocin + fluorouracil; |
| | | chlorozotocin^{†}; octreotide |
| Kaposi's sarcoma* (Aids-related) | Etoposide or interferon alfa or vinblastine | Vincristine, Doxorubicin, |
| | Doxorubicin + bleomycin + vincristine or | bleomycin |
| | vinblastine (ABV) | |
| Leukemia | | |
| Acute lymphocytic leukemia | Induction: Vincristine + prednisone + asparaginase ± daunorubicin | Induction: same ± high-dose |
| (ALL)⁷ | CNS prophylaxis: Intrathecal methotrexate ± systemic | methotrexate ± cyterabine; |
| | high-dose methotrexate with | pegaspargase instead of |
| | leutovorin ± Intrathecal cytarabine ± Intrathecal | asparaginese |
| | hydrocortisone | Teniposide or etoposide |
| | Maintenance: Methotrexate + mercaptopurine | High-dose cytarabine |
| | Bone marrow transplant.³⁸ | Maintenance: same + periodic |
| | | vincristine + prednisone |
| Acute myeloid leukemia (AML)⁹ | Induction: Cytsrabine + either daunorubicin | Cytarabine + mitoxentrone |
| | or idarubicin | High-dose cyterabine |
| | Post Induction: High-dose cytarabine ± other | |
| | drugs such as etoposide | |
| | Bone marrow transplant³. | |
| Chronic lymphocytic leukemia | Chlorambucil ± prednisone | Cladribine, cyclophosphamide, |
| (CLL) | Fludarabin | pentostatin, vincristine, |
| | | Doxorubicin |
| Chronic myeloid leukemia | | |
| (CML)¹⁰ | | |
| Chronic phase | Bone marrow transplant³ | Busulfan |
| | Interferon alfa | |
| | Hydroxyures | |
| Accelerated¹¹ | Bone marrow transplant³ | Hydroxyures, busulfen |
| Blast crisis¹¹ | Lymphoid: Vincristine + prednisone + L- | Tretinoln^{†} |
| | separaginess + intrathecal methotrexate (± maintenance | Amsecrine, ^{†}azacitidine |
| | with methotrexate + 8- | Vincristine ± plicamycin |
| | marcaptopurine) | |
| Hairy cell Leukemia | Pentostatin or cladribine | Interferon alfa, chlorambucil, |
| | | fludarabin |
| Liver** | Doxorubicin | Intrahepatic-arterial floxuridine |
| | Fluorouracil | or claplatin |
| Lung, small cell (cat cell) | Claplatin + etoposide (PE) | Ifosfamide with means + |
| | Cyclophosphamide + doxorubicin + vincristine | carboplatin + etoposide (ICE) |
| | (CAV) | Daily oral etoposide |
| | PE alternated with CAV | Etoposide + ifosfamide with |
| | Cyclophosphamide + etoposide + claplatin | means + claplatin (VIP |
| | (CEP) | Paclitaxel |
| | Duxorubicin + cyclophosphamide + etoposide | |
| | (ACE) | |
| Lung | Claplatin + etoposide | Claplatin + fluorouracil + leucovorin |
| (non-small cell)** | Claplatin + Vinblastine ± mitomycin | Carboplatin + paclitaxel |
| | Claplatin + vincrisine | |
| Lymphomas | | |
| Hodgkin's¹² | Doxorubicin + bleomycin + vinblastine + | Mechlorethamine + vincristine + |
| | dacarbazine (ABVD) | procarbazine + prednisone (MOPP) |
| | ABVD alternated | Chlorambusil + vinblastine + |
| | with MOPP | procarbazine + prednisone ± carmustine |
| | Mechlorethamine + vincristine + procarbazine | Etoposide + vinblastine + doxorubicin |
| | (± prednisone) + doxorubicin + bleomycin + vinblastine | Bone marrow transplant³ |
| | (MOP[P]-ABV) | |
| Non-Hodgkin's | | |
| Burkitt's lymphoma | Cyclophosphamide + vincristine + methotrexate | Ifosfamide with means |
| | Cyclophosphamide + high-dose | Cyclophosphamide + doxorubicin + |
| | cytarabine ± methotrexate | vincrletine + prednisone |
| | with leutovorin | (CHOP) |
| | Intrathecal methotrexate or cytarabine | |
| Difuse large-cell lymphoma | Cyclophosphamide + doxorubicin + vincristine + prednisone | Dexamethasone sometimes |
| | (CHOP) | substituted for prednisone |
| | | Other combination regimens, |
| | | which may include methotrexate, |
| | | etoposide, cytarabine, |
| | | bleomycin, procarbazine, |
| | | ifosfamide and mitoxantrone |
| | | Bone marrow transplant³ |
| Follicular lymphoma | Cyclophosphamide or chlorambusil | Same ± vincristine and |
| | | prednisone, ± etoposide |
| | | Interferon alfa, cladribine, |
| | | fludarabin |
| | | Bone marrow transplant³ |
| | | Cyclophosphamide + doxorubicin + |
| | | vincristine + prednisone |
| | | (CHOP) |
| Melanoma** | Interferon Alfa | Carmustine, lomustine, cisplatin |
| | Dacarbazine | Dacarbazine + clapletin + |
| | | carmustine + tamoxifen |
| | | Aldesleukin |
| Mycosis fungoides* | PUVA (psoralen + ultraviolet A) | Isotretinoin, topical carmustine, |
| | Mechlorethamine (topical) | pentosistin, fludarabin, |
| | Interferon alfa | cladribine, photopheresis (extra- |
| | Electron beam radiotherapy | corporeal photochemitherapy), |
| | Methotrexate | chemotherapy as in non- |
| | | Hodgkin's lymphoma |
| Mysloma* | Melphelan (or cyclophosphamide) + prednisons | Interferon alfa |
| | Melphalan ± carmustine + cyclophosphamide + prednisons + vincristine | Bone marrow transplant³ |
| | Dexamethasone + doxorubicin + vincristine | High-dose dexamethasons |
| | (VAD) | |
| | Vincristine + carmustine + doxorubicin + prednisons | |
| | (VBAP) | |
| Neuroblestoma* | Doxorubicin + cyclophosphamide + claplatin + teniposide | Carboplatin, etoposide |
| | or etoposide | Bone marrow transplant³ |
| | doxorubicin + cyclophosphamide | |
| | Claplatin + cyclophosphamide | |
| Osteogenic sarcoma⁵ | Doxorubicin + claplatin ± etopside ± ifosfamide | Ifosfamide with means, |
| | | etoposide, carboplatin, high- |
| | | dose methotrexate with |
| | | leucovorin |
| | | Cyclophosphamide + etoposide |
| Ovary | Claplatin (or carboplatin) + paclitaxel | Ifosfamide with means, |
| | Claplatin (or carboplatin) + cyclophosphamide | paclitaxel, tamoxifen, |
| | (CP) ± doxorubicin | melphalan, altretamine |
| | (CAP) | |
| Pancreatic** | Fluoroutacil ± leucovorin | Gemoltabinet |
| Prostate | Leuprolide (or goserelln) ± flutamide | Estramustine ± vinblastine, |
| | | aminoglutethimide + hydrocortleone, |
| | | estramustine + etoposide, |
| | | diethylstllbestrol, nilutamide |
| Renal** | Aldesleukin | Vinblastine, floxuridine |
| | Inteferon alfa | |
| Retinoblestoma^{5*} | Doxorubicin + cyclophosphamide ± claplatin ± etoposide ± vincristina | Carboplatin, etoposide, |
| | | Ifosfamide with means |
| Sarcomas, soft tissue, adult* | Doxorubicin ± decarbazine ± cyclophosphamide ± Ifosfamide | Mitornyeln + doxorubicin + claplatin |
| | with | Vincristina, etoposide |
| | means | |
| Testicular | Claplatin + etoposide ± bleomycin | Vinblestine (or etoposide) + Ifosfamide |
| | (PEB) | with means + claplatin |
| | | (VIP) |
| | | Bone marrow transplant³ |
| Wilms' tumor⁵ | Dectinomycln + vincriatine ± doxorubicin ± cyclophosphamide | Ifosfamide with means, |
| | | etoposide, carboplatin |

*Chemotherapy has only moderate activity.
**Chemotherapy has only minor activity.
¹Tamoxifen with or without chemotherapy is generally recommended for postmenopausal estrogen-receptor-positive,
mode-positive patients and chemotherapy with or without tamoxlfen for premenopausal mode-positive patients. Adjuvant treatment
with chemotherapy and/or tamoxifen is recommended for mode-negative patients with larger tumors or other adverse prognostic indicators.
²Megastrol and other hormonal agents may be effective in some patients with tamoxifen fails.
³After high-dose chemotherapy (Medical Letter, 34: 79, 1982).
⁴For rectal cancer, postoperative adjuvant treatment with fluoroutacil plus radiation, preceded and followed by treatment
with fluorouracil alone.
⁵Drugs have major activity only when combined with surgical resection, radiotherapy or both.
⁶The vitamin A analog lactratinoln (Acgutana) can control pre-neoplastic lesions (leukoplakla) and decreases the rate of
second primary tumors (Banner et al, 1994).
^{†}Available in the USA only for investigational use.
⁷High-risk patients (e.g., high counts, cytogenetic abnormalities, adults) may require additional drugs for induction,
maintenance and “Intensificiation” (use of
additional drugs after achievement of remission). Additional drugs include
cyclophosphamida, mitoxantrone and thloguanine. The results of one large controlled
trial in the United Kingdom suggest that Intensificiation may improve survival in all
children with ALL (Chasselle et al, 1995).
⁸Patients with a poor prognosis initially or those who relapse after remission.
⁹Some patients with acute promyelocytic leukemia have had complete responses to
tratinoin. Such treatment can cause a toxic syndrome characterized primarily by fever
and respiratory distress (Warrell, Jr et al, 1993).
¹⁰Allogeheic HLA-identical sibling bone marrow transplantation can cure 40% to
70% of patients with CML in chronic phase, 18% to 28% of patients with accelerated
phase CML, and <15% patients in blast crisis. Disease-free survival after bone marrow
transplantations adversely influenced by age >50 years, duration of disease >3 years
from diagnosis, and use of one-antigen-mismatched or matched-unrelated donor marrow.
Interferon also may be curative in patients with chronic phase CML who achieve a
complete cytogenetic response (about 10%); it is the treatment of choice for patents >80
years old with newly diagnosed chronic phase CML and for all patients who are not
candidates for an allgensic bone marrow transplant. Chemotherapy alone is palliative.
¹¹If a second chronic phase is achieved with any of these combinations, allogeneic
bone marrow transplant should be considered. Bone marrow transplant in second chronic
phase may be curative for 30% to 35% of patients with CML.
¹²Limited-stage Hodgkin's disease (stages 1 and 2) is curable by radiotherapy.
Disseminated disease (stages 3b and 4) require chemotherapy. Some intermediate stages
and selected clinical situations may benefit from both.
+ Available in the USA only for investigational use.

ANTICANCER DRUGS AND HORMONES

| Drug | Acute Toxicity‡ | Delayed toxicity‡ |
|---|---|---|
| Aldesleukin (Interleukin-2; | Fever; fluid retention; hypertension; | Neuropsychiatric disorders; |
| Proleukin - Cetus | respiratory distress; rash; anemia; | hypothyrldiam; nephrotic |
| Oncology) | thrombocytophenia; nausea and vomiting; | syndrome; possibly acute |
| | diarrhea; capillary leak syndrome; | leukoencaphalopathy; brachial |
| | naphrotoxlolty; myocardial toxicity; | plexopathy; bowel perforation |
| | hepatotoxicity; erytherna nodosum; | |
| | neutrophil chemotactic defects | |
| Altretamine (hexamethyl- | Nausea and vomiting | Bone marrow depression; CNS |
| melamine; Hexalen - U | | depression; peripheral |
| Bioscience) | | neuropathy; visual |
| | | hallucinations; stexis; tremors, |
| | | alopecia; rash |
| Aminogiutethimide (Cytadren - | Drowsiness; nausea; dizziness; rash | Hypothryroidism (rare); bone |
| Ciba) | | marrow depression; fever; |
| | | hypotension; mascullinization |
| †Amsacrine (m-AMSA; amaidine; | Nausea and vomiting; diarrhea; pain or | Bone marrow depression; |
| AMSP P-D-Parke-Davis, | phlebitis on infuelon; anaphylaxia | hepactic injury; convulsions; |
| Amsidyl-Warner- | | stomatitle; ventricular |
| Lambert) | | fibrillation; alopecia; congestive |
| | | heart failure; renal dysfunction |
| Asparaginase (Elspar-merck; | Nausea and vomiting; fever; chills; headache; | CNS depression or |
| Kidrolase in Canada) | hypersensitivity, anaphylexia; abdominal | hyperexcitability; acute |
| | pain; hyperglycemia leading to coma | hemorrhagic pancreatitis; |
| | | coagulation defects; thromboals; |
| | | renal damage; hepactic damage |
| Cervix** | Claplatin Ifosfamide with means | Chlorambucil, vincristine, |
| | Bleomycin patin | fluoroutacil, doxorubicin, |
| | Ifosfamide with means | methotrexete, altretamine |
| Chorlocarcinoma | Methotrexete ± leucovorin Dactinomyclin | Methotrexete + dectinomycin + |
| | | cyclophosphamide (MAC) |
| | | Etoposide + methotrexate + dactinomycin + |
| | | cyclophosphamide + vincrlatine |
| Colorectal* | Adjuvant colon⁴: Fluoroutacil + lavamleole; | Hepatic metastases: |
| | fluoroutacil + leucovarin Metastatic: | Intrahepactic-arterial floxuridine |
| | Fluoroutacil + leucvarin | Mitomyclin |
| Embryonal rhebdomyosarcoma⁶ | Vincriatine + dectinomycin ± cyclophosphamide | Same + doxorubicin |
| | Vincristine + Ifosfamide with means + etoposide | |
| Endometrial** | Megastrol or another progeetin | Fluoroutacil, tamoxifen, |
| | Doxorubicin + claplatin ± cyclophosphamide | altretamine |
| Cancer | Drugs of Choice | Some alternatives |
| Esophageal* | Claplatin + Fluoroutacil | Doxorubicin, methotrexete, |
| Ewing's sarcoma⁵ | Cyclophosphamide (or ifosfamide with | mitomycin |
| | means) + doxorubicin + vincrietine (CAV) ± dectinomycin | CAV + etoposide |
| Gastric** | Fluoroutacil ± leucovoin | Claplatin, doxorubicin, |
| | | etoposide, methotrexete + leucovorin, |
| | | mitomycin |
| Head and neck squamous cell*⁵ | Claplatin + fluoroutacil | Blaonycin, carboplatin, |
| Islet call** | Methotrexete | paciltaxel |
| | Streptozocin + doxorubicin | Streptozocln + fluoroutacil; |
| | | chlorozotocin; actreatide |
| Kaposal's sercoma* | Etoposide or Interferon alfa or vinbleomycin | Vincristine, doxorubicin, |
| (AIDS-related) | stine | bleomycln |
| | Doxorubicin + bleomycin + vincristine or | |
| | vinbleomycin stine (ABV) | |
| Leukemias | Induction: Vincristine + prednisone + asparaginase ± daunorubieln | Industion: same ± high-dose |
| Acute lymphocytic leukemia | CNS prophylaxia; Intrathecal methotrexete ± systemic | methotrexete ± cyterabine; |
| (ALL)⁷ | high-dose methotrexete with | pegaspargase instead of |
| | leucovorin ± Intrethecal cytarabine ± Intrathecal | aspareginese |
| | hydrocortisone | Teniposide or etoposide |
| | Maintenance: methotrexete ± mercaptopurine | High-dose cytarabine |
| | Bone marrow transplant³ | Maintenance: same + periodic |
| | | vincristine + prednisone |
| Acute myeloid leukemia (AML)⁹ | Induction: Cytarabine + either daunbrublein | Cytarabine + mitoxantrone |
| | or idarubieln | High-dose cytarabine |
| | Post Induction: High-dose cytarabine ± other | |
| | drugs such as etoposide | |
| | Bone marrow transplant³ | |
| Chronic lymophocytic leukemia | Chlorambuell ± prednisone | Claplatin, cyclophosphamide, |
| (CLL) | Fludarabin | pentostatin, vinorlstine, |
| | | doxorubicin |

†Available in the USA only for investigational use.
‡Dose-limiting effects are in bold type. Cutaneous reactions (sometimes severe), hyperpigmentation, and ocular toxicity have
been reported with virtually all nonhormonal anticancer drugs. For adverse interactions with other drugs, see the Medical Letter
Handbook of Adverse Drug Interactions, 1995.
¹Available in the USA only for investigational use.
²Megestrol and other hormonal agents may be effective in some pateients when tamoxifen fails.
³After high-dose chemotherapy (Medical Letter, 34:78, 1992).
⁴For rectal cancer, postoperative adjuvant treatment with fluoroutacil plus radiation, preceded and followed by treatment with
fluoroutacil alone.
⁵Drugs have major activity only when combined with surgical resection, radiotherapy or both.
⁶The vitamin A analog isotretinoin (Accutane) can control pre-neoplastic isions (leukoplaka) and decreases the rats of second
primary tumors (Senner et al., 1994).
⁷High-risk patients (e.g., high counts, cytogenetic abnormalities, adults) may require additional drugs for Induction, maintenance
and “Intensification” (use of additional drugs after achievement of remission). Additional drugs include cyclophosphamide,
mitoxantrone and thioguamine. The results of one large controlled trial in the United Kingdom suggest that intensilibation may
improve survival in all children with ALL Chassella et al., 1995).
⁸Patients with a poor prognosis initially or those who relapse after remission
⁹Some patients with acute promyclocytic leukemia have had complete responses to tretinoin. Such treatment can cuase a toxic
syndrome characterized primarily by fever and respiratory distress (Warrell, Jr et al. 1993).
¹⁰Allogenaic HLA Identical sibling bone marrow transplantation can cure 40% to 70% of patients with CML in chroni phase, 15%
to 25% of patients with accelerated phase CML, and <15% patients in blast crisis. Disease-free survival after bone marrow
transplantation is adversely influenced by age >50 years, duration of disease >3 years from diagnosis, and use of one antigen
mismatched or matched-unrelated donor marrow. Inteferon alfa may be curative in patients with chronic phase CML who
achieve a complete cytogenetic resonse (about 10%); It is the treatment of choices for patients >50 years old with newly
diagnosed chronic phase CML and for all patients who are not candidates for an allogenic bone marrow transplant.
Chemotherapy alone is palliative.

D. Diagnostic Moieties

In certain embodiments of the compositions of the present invention, a diagnostic moiety is bound to the polypeptide. As defined herein, a “diagnostic moiety” is a part of a molecule that is a chemical or compound that can be administered to a subject or contacted with a tissue for the purpose of facilitating diagnosis of a disease or disorder, or condition associated with abnormal cell physiology. Any diagnostic agent known to those of ordinary skill in the art is contemplated as a diagnostic moiety. In certain embodiments, the diagnostic moiety is a diagnostic amino acid sequence that is chemically conjugated or fused to a polypeptide that is capable of binding to a valent metal ion.

One example of a diagnostic moiety would be an imaging moiety. As defined herein, an “imaging moiety” is a part of a molecule that is a agent or compound that can be administered to a subject, contacted with a tissue, or applied to a cell for the purpose of facilitating visualization of particular characteristics or aspects of the subject, tissue, or cell through the use of an imaging modality. Imaging modalities are discussed in greater detail below. Any imaging agent known to those of ordinary skill in the art is contemplated as an imaging moiety of the present invention. Thus, for example, in certain embodiments of the compositions of the present invention, the compositions can be applied in multimodality imaging techniques. Dual imaging and multimodality imaging are discussed in greater detail in the specification below.

In certain embodiments, the imaging moiety is a contrast media. Examples include CT contrast media, MRI contrast media, optical contrast media, ultrasound contrast media, or any other contrast media to be used in any other form of imaging modality known to those of ordinary skill in the art. Examples include diatrizoate (a CT contrast agent), a gadolinium chelate (an MRI contrast agent), and sodium fluorescein (an optical contrast media). Additional examples of contrast media are discussed in greater detail in the specification below. One of ordinary skill in the art would be familiar with the wide range of types of imaging agents that can be employed as imaging moieties in the polypeptides of the present invention.

E. Tissue-Targeting Moieties

In some embodiments of the compositions of the present invention, a second moiety is bound to the polypeptide, wherein the second moiety is a tissue-targeting moiety. A “tissue-targeting moiety” is defined herein to refer to a part of a molecule that can bind or attach to tissue. The binding may be by any mechanism of binding known to those of ordinary skill in the art. Examples include antimetabolites, apoptotic agents, bioreductive agents, signal transductive therapeutic agents, receptor responsiveagents, or cell cycle specific agents. The tissue may be any type of tissue, such as a cell. For example, the cell may be the cell of a subject, such as a cancer cell. In certain embodiments, the tissue targeting moiety is a tissue targeting amino acid sequence that is chemically conjugated or fused to a polypeptide that is capable of binding to a valent metal ion.

In some embodiments the tissue-targeting moiety is a “targeting ligand.” A “targeting ligand” is defined herein to be a molecule or part of a molecule that binds with specificity to another molecule. One of ordinary skill in the art would be familiar with the numerous agents that can be employed as targeting ligands in the context of the present invention.

Examples of targeting ligands include disease cell cycle targeting compounds, tumor angiogenesis targeting ligands, tumor apoptosis targeting ligands, disease receptor targeting ligands, drug-based ligands, antimicrobials, tumor hypoxia targeting ligands, an agent that mimics glucose, amifostine, angiostatin, EGF receptor ligands, capecitabine, COX-2 inhibitors, deoxycytidine, fullerene, herceptin, human serum albumin, lactose, leuteinizing hormone, pyridoxal, quinazoline, thalidomide, transferrin, and trimethyl lysine.

In further embodiments of the present invention, the tissue-targeting moiety is an antibody. Any antibody is contemplated as a tissue-targeting moiety in the context of the present invention. For example, the antibody may be a monoclonal antibody. One of ordinary skill in the art would be familiar with monoclonal antibodies, methods of preparation of monoclonal antibodies, and methods of use of monoclonal antibodies as ligands. In certain embodiments of the present invention, the monoclonal antibody is an antibody directed against a tumor marker. In some embodiments, the monoclonal antibody is monoclonal antibody C225, monoclonal antibody CD31, or monoclonal antibody CD40.

A single tissue-targeting moiety, or more than one such tissue-targeting moiety, may be bound to a polypeptide of the present invention. In these embodiments, any number of tissue-targeting moieties may be bound to the polypeptides set forth herein. Thus, there may be one or more tissue targeting moieties attached to a polypeptide of the present invention. The tissue-targeting moieties can be bound to the polypeptide in any manner. For example, the tissue-targeting moiety may be bound to the polypeptide in an amide linkage, or in an ester linkage. One of ordinary skill in the art would be familiar with the chemistry of these agents, and methods to incorporate these agents as moieties of the polypeptides of the claimed invention. Methods of synthesis of the compounds of the present invention are discussed in detail below.

Information pertaining to tissue targeting moieties and conjugation with compounds are provided in U.S. Pat. No. 6,692,724, U.S. patent application Ser. No. 09/599,152, U.S. patent application Ser. No. 10/627,763, U.S. patent application Ser. No. 10/672,142, U.S. patent application Ser. No. 10/703,405, U.S. patent application Ser. No. 10/732,919, each of which is herein specifically incorporated by reference in their entirety for this section of the specification and all other sections of the specification.

Representative examples of tissue-targeting moieties are discussed below.

1. Disease Cell Cycle Targeting Compounds

Disease cell cycle targeting refers to targeting of agents that are upregulated in proliferating cells. “Disease cell cycle targeting compounds” are compounds that are used to measure agents that are upregulated or downregulated in proliferating cells. For example, the cells may be cancer cells. Compounds used for this purpose can be used to measure various parameters in cells, such as tumor cell DNA content.

Many of these agents are nucleoside analogues. For example, pyrimidine nucleoside (e.g., 2′-fluoro-2′-deoxy-5-iodo-1-β-D-arabinofuranosyluracil [FIAU], 2′-fluoro-2′-deoxy-5-iodo-1-β-D-ribofuranosyl-uracil [FIRU], 2′-fluoro-2′-5-methyl-1-β-D-arabinofuranosyluracil [FMAU], 2′-fluoro-2′-deoxy-5-iodovinyl-1-β-D-ribofuranosyluracil [IVFRU]) and acycloguanosine: 9-[(2-hydroxy-1-(hydroxymethyl)ethoxy)methyl]guanine (GCV) and 9-[4-hydroxy-3-(hydroxy-methyl)butyl]guanine (PCV) (Tjuvajev et al., 2002; Gambhir et al., 1998; Gambhir et al., 1999) and other ¹⁸F-labeled acycloguanosine analogs, such as 8-fluoro-9-[(2-hydroxy-1-(hydroxymethyl)ethoxy)methyl]guanine (FGCV) (Gambhir et al., 1999; Namavari et al., 2000), 8-fluoro-9-[4-hydroxy-3-(hydroxymethyl)butyl]guanine (FPCV) (Gambhir et al., 2000; Iyer et al., 2001), 9-[3-fluoro-1-hydroxy-2-propoxy methyl]guanine (FHPG) (Alauddin et al., 1996; Alauddin et al., 1999), and 9-[4-fluoro-3-(hydroxymethyl)butyl]guanine (FHBG) (Alauddin and Conti, 1998; Yaghoubi et al., 2001) have been developed as reporter substrates for imaging wild-type and mutant (Gambhir et al., 2000) HSV1-tk expression. Particular embodiments of the compounds of the present invention include adenosine and penciclovir (guanine) as the disease cell cycle targeting ligand. One or ordinary skill in the art would be familiar with these and other agents that are used for disease cell cycle targeting.

2. Angiogenesis Targeting Ligands

“Angiogenesis targeting ligands” refers to agents that can bind to neovascularization, such as neovascularization of tumor cells. Agents that are used for this purpose are known to those of ordinary skill in the art for use in performing various tumor measurements, including measurement of the size of a tumor vascular bed, and measurement of tumor volume. Some of these agents bind to the vascular wall. One of ordinary skill in the art would be familiar with the agents that are available for use for this purpose.

Throughout this application, “tumor angiogenesis targeting” refers to the use of an agent to bind to tumor neovascularization and tumor cells. Agents that are used for this purpose are known to those of ordinary skill in the art for use in performing various tumor measurements, including measurement of the size of a tumor vascular bed, and measurement of tumor volume. Some of these agents bind to the vascular wall. One of ordinary skill in the art would be familiar with the agents that are available for use for this purpose. A tumor angiogenesis targeting ligand is a ligand that is used for the purpose of tumor angiogenesis targeting as defined above. Examples include COX-2 inhibitors, anti-EGF receptor ligands, herceptin, angiostatin, C225, and thalidomide. COX-2 inhibitors include, for example, celecoxib, rofecoxib, etoricoxib, and analogs of these agents.

3. Tumor Apoptosis Targeting Ligands

“Tumor apoptosis targeting” refers to use of an agent to bind to a cell that is undergoing apoptosis or at risk of undergoing apoptosis. These agents are generally used to provide an indicator of the extent or risk of apoptosis, or programmed cell death, in a population of cells, such as a tumor. One of ordinary skill in the art would be familiar with agents that are used for this purpose. A “tumor apoptosis targeting ligand” is a ligand that is capable of performing “tumor apoptosis targeting” as defined in this paragraph. The targeting ligand of the present invention may include TRAIL (TNF-related apoptosis inducing ligand) monoclonal antibody. TRAIL is a member of the tumor necrosis factor ligand family that rapidly induces apoptosis in a variety of transformed cell lines. The targeting ligand of the present invention may also comprise a substrate of caspase-3, such as peptide or polypeptide that includes the 4 amino acid sequence aspartic acid-glutamic acid-valine-aspartic acid. caspase-3 substrate (for example, a peptide or polypeptide that includes the amino acid sequence aspartic acid-glutamic acid-valine-aspartic acid), and any member of the Bcl family. Examples of Bcl family members include, for example, Bax, Bc1-xL, Bid, Bad, Bak, and Bc1-2). One of ordinary skill in the art would be familiar with the Bc1 family, and their respective substrates.

Apoptosis suppressors are targets for drug discovery, with the idea of abrogating their cytoprotective functions and restoring apoptosis sensitivity to tumor cells (Reed, 2003).

4. Disease Receptor Targeting Ligands

In “disease receptor targeting,” certain agents are exploited for their ability to bind to certain cellular receptors that are overexpressed in disease states, such as cancer. Examples of such receptors which are targeted include estrogen receptors, androgen receptors, pituitary receptors, transferrin receptors, and progesterone receptors. Examples of agents that can be applied in disease-receptor targeting include androgen, estrogen, somatostatin, progesterone, transferrin, luteinizing hormone, and luteinizing hormone antibody.

The radiolabeled ligands, such as pentetreotide, octreotide, transferrin, and pituitary peptide, bind to cell receptors, some of which are overexpressed on certain cells. Since these ligands are not immunogenic and are cleared quickly from the plasma, receptor imaging would seem to be more promising compared to antibody imaging.

The folate receptor is included herein as another example of a disease receptor. Folate receptors (FRs) are overexposed on many neoplastic cell types (e.g., lung, breast, ovarian, cervical, colorectal, nasopharyngeal, renal adenocarcinomas, malign melanoma and ependymomas), but primarily expressed only several normal differentiated tissues (e.g., choroid plexus, placenta, thyroid and kidney) (Weitman et al., 1992a; Campbell et al., 1991; Weitman et al., 1992b; Holm et al., 1994; Ross et al., 1994; Franklin et al., 1994; Weitman et al., 1994). FRs have been used to deliver folate-conjugated protein toxins, drug/antisense oligonucleotides and liposomes into tumor cells overexpressing the folate receptors (Ginobbi et al., 1997; Leamon and Low, 1991; Leamon and Low, 1992; Leamon et al., 1993; Lee and Low, 1994). Furthermore, bispecific antibodies that contain anti-FR antibodies linked to anti-T cell receptor antibodies have been used to target T cells to FR-positive tumor cells and are currently in clinical trials for ovarian carcinomas (Canevari et al., 1993; Bolhuis et al., 1992; Patrick et al., 1997; Coney et al, 1994; Kranz et al., 1995).

Examples of folate receptor targeting ligands include folic acid and analogs of folic acid. Preferred folate receptor targeting ligands include folate, methotrexate and tomudex. Folic acid as well as antifolates such as methotrexate enter into cells via high affinity folate receptors (glycosylphosphatidylinositol-linked membrane folate-binding protein) in addition to classical reduced-folate carrier system (Westerhof et al., 1991; Orr et al., 1995; Hsuch and Dolnick, 1993).

5. Drug Assessment

Certain drug-based ligands can be applied in measuring the pharmacological response of a subject to a drug. A wide range of parameters can be measured in determining the response of a subject to administration of a drug. One of ordinary skill in the art would be familiar with the types of responses that can be measured. These responses depend in part upon various factors, including the particular drug that is being evaluated, the particular disease or condition for which the subject is being treated, and characteristics of the subject. Examples of drug-based ligands include carnitine and puromycin.

6. Antimicrobials

Any antimicrobial is contemplated for inclusion as a targeting ligand. Preferred antimicrobials include ampicillin, amoxicillin, penicillin, cephalosporin, clidamycin, gentamycin, kanamycin, neomycin, natamycin, nafcillin, rifampin, tetracyclin, vancomycin, bleomycin, and doxycyclin for gram positive and negative bacteria and amphotericin B, amantadine, nystatin, ketoconazole, polymycin, acyclovir, and ganciclovir for fungi. One of ordinary skill in the art would be familiar with the various agents that are considered to be antimicrobials.

7. Agents that Mimic Glucose

Agents that mimic glucose are also contemplated for inclusion as targeting ligands. Preferred agents that mimic glucose, or sugars, include neomycin, kanamycin, gentamycin, paromycin, amikacin, tobramycin, netilmicin, ribostamycin, sisomicin, micromicin, lividomycin, dibekacin, isepamicin, astromicin, aminoglycosides, glucose or glucosamine.

8. Tumor Hypoxia Targeting Ligands

In some embodiments of the present invention, the targeting ligand is a tumor hypoxia targeting ligand. Tumor cells are more sensitive to conventional radiation in the presence of oxygen than in its absence; even a small percentage of hypoxic cells within a tumor could limit the response to radiation (Hall, 1988; Bush et al., 1978; Gray et al., 1958). Hypoxic radioresistance has been demonstrated in many animal tumors but only in few tumor types in humans (Dische, 1991; Gatenby et al., 1988; Nordsmark et al., 1996). The occurrence of hypoxia in human tumors, in most cases, has been inferred from histology findings and from animal tumor studies. In vivo demonstration of hypoxia requires tissue measurements with oxygen electrodes and the invasiveness of these techniques has limited their clinical application.

Misonidazole, an example of a tumor hypoxia targeting ligand, is a hypoxic cell sensitizer, and labeling MISO with different radioisotopes (e.g., ¹⁸F, ¹²³I, ^{99m}Tc) may be useful for differentiating a hypoxic but metabolically active tumor from a well-oxygenated active tumor by PET or planar scintigraphy. [¹⁸F]Fluoromisonidazole (FMISO) has been used with PET to evaluate tumors hypoxia. Recent studies have shown that PET, with its ability to monitor cell oxygen content through [¹⁸F]FMISO, has a high potential to predict tumor response to radiation (Koh et al., 1992; Valk et al., 1992; Martin et al., 1989; Rasey et al., 1989; Rasey et al., 1990; Yang et al., 1995). PET gives higher resolution without collimation, however, the cost of using PET isotopes in a clinical setting is prohibitive.

F. Methods of Synthesis

1. Source of Reagents for the Compositions of the Present Invention

Reagents for preparation of the compositions of the present invention can be obtained from any source. A wide range of sources are known to those of ordinary skill in the art. For example, the reagents can be obtained from commercial sources, from chemical synthesis, or from natural sources. The reagents may be isolated and purified using any technique known to those of ordinary skill in the art. For example, polynucleotides of a particular molecular weight can be isolated using particular dialysis membranes. Examples of valent metal ions to be employed in the compositions of the present invention include valent metal ions obtained from generators (e.g., Tc-99m, Cu-62, Cu-67, Ga-68, Re-188, Bi-212), cyclotrons (e.g., Cu-60, Cu-61, As-72, Re-186) and commercial sources (e.g., In-111, Tl-201, Ga-67, Y-90, Sm-153, Sr-89, Gd-157, Ho-166). The free unbound metal ions can be purified with ion-exchange resin or by adding a transchelator (e.g., glucoheptonate, gluconate, glucarate, and acetylacetonate). One of ordinary skill in the art would be familiar with methods of purification, including use of ion-exchange resins and transchelators.

2. Coordination of a Valent Metal Ion to a Polypeptide

Valent metal ions such as gadolinium, gallium, rhenium, technetium or platinum will be chelated to polypeptide without chelators. The reaction can be carried out in an aqueous medium or a nonaqueous medium. Most preferably, the conjugation is carried out in an aqueous medium.

In some embodiments, the valent metal ion is chelated to one of the acid groups of the two more more consecutive amino acids selected from the group consisting of glutamate, aspartate, an analog of glutamate, an analog of aspartate, and a non-naturally occurring amino acid that includes two more more carboxyl groups. In certain particular embodiments, the valent metal ion is chelated to 4 or 5 carboxyl groups of a series of consecutive glutamate or aspartate residues.

Any method of coordinating a valent metal ion to the polypeptide known to those of ordinary skill in the art can be applied in the present invention. In some embodiments of the present invention, for example, the polypeptide is dissolved in water, and then tin (II) chloride solution added. The valent metal ion (e.g., Na ^{99m}TcO₄ or Na ^{186/188}ReO₄) can then be added. Other metals (gallium chloride, gadolinium chloride, copper chloride, cobolt chloride, platinum) may not require tin (II) chloride solution. Any method known to those of ordinary skill in the art can be used to measure radiochemical purity. For example, it may be measured using thin layer chromatography (TLC) eluted with methanol:ammonium acetate (1:4).

Any method known to those of ordinary skill in the art can be used to isolate the valent metal ion-polypeptide conjugate from solution. For example, in some embodiments, the reaction mixture can be purified by dialysis and evaporated to dryness, and then later reconstituted in water for use.

3. Conjugation of a Second Moiety to a Polypeptide

Any method known to those of ordinary skill in the art can be used to conjugate a diagnostic moiety, therapeutic moiety, or tissue targeting moiety to the polypeptide. For example, the second moiety can be conjugated to a carboxyl group of a glutamate or aspartate residue of the polypeptide to form a carboxylate-metal ion complex.

Any ratio of reagents can be used in the reaction mixture. For example, in some embodiments the ratio of polypeptide to moiety is 1:1 in water. The different ratio may change the solubility and viscosity in aqueous solution. In some embodiments of the present methods, a coupling agent is used to couple a second moiety to a polypeptide. In certain embodiments, the coupling agent used in aqueous condition is 1-ethyl-3-(3-dimethylaminopropyl) carbodiimide-HCl (EDC). Other examples of the coupling agent used in nonaqueous condition is 1,3-dicyclohexylcarbodiimide (DCC). In some embodiments of the present methods, the second moiety can first be dissolved in water. The aqueous solution comprising the second moiety can then be added to an aqueous solution comprising the polypeptpide. The reaction mixture can then be stirred for 25 hours at room temperature. The product can then be isolated from solution by any method known to those of ordinary skill in the art. For example, the product can be dialyzed from solution using a dialysis membrane that has a cut-off at 1,000 daltons. The product can then be used immediately, or freeze-dried and stored.

Conjugation of the second moiety can be to any residue of the polypeptide. In certain preferred embodiments, the conjugation is to an acid group of the polypeptide. The polypeptide can include a single second moiety, or multiple second moieties. In certain particular embodiments of the present invention, each carboxyl group of the polypeptide is either conjugated to a second moiety or coordinated to a valent metal ion.

More than one type of second moiety can be conjugated to a particular polypeptide. For example, in some embodiments, a therapeutic and tissue targeting moiety are conjugated to a single polypeptide. Therapeutic agents, such as methotrexate or doxorubicin, can be conjugated the amino or acid moieties of the polypeptide. Diagnostic agents such as diatrizoic acid, iothalmic acid, and iopanoic acid can be conjugated to amino or acid moieties of the polypeptide. Tissue targeting moieties such as hypoxic markers (metronidazole, misonidazole), glycolysis markers (sugar), amino acids (e.g., tyrosine, lysine), cell cycle markers (e.g., adenosine, guanosine), or receptor markers (e.g., estrogen, folate, androgen) can be conjugated to the amino or acid moieties of the polypeptide. In particular embodiments, conjugation is to acid moieties of the polypeptide. In other embodiments, two or more different therapeutic or diagnostic moieties are conjugated to the same polypeptide. For example, in certain embodiments, a diagnostic agent (e.g., x-ray contrast media or optical contrast media) and a radiometallic substance are conjugated to the same polypeptide. It may be employed for PET/CT, SPECT/CT, or optical/CT applications. In further embodiments, a non-radioactive metallic substance (e.g., gadolinium, iron, or manganse) is coordinated to the polypeptide. It may be employed in any forms of imaging, including PET/MRI, SPECT/MRI, or optical/MRI applications. In still further embodiments, a therapeutic agent and a radiotherapeutic metallic substance are conjugated to the same polypeptide. Such agents may be employed for radiochemotherapy.

4. Generation of Chimeric Polypeptides

Certain embodiments of the present invention generally pertain to compositions that include a polypeptide that includes within its sequence (a) a tissue targeting amino acid sequence, a diagnostic amino acid sequence, and/or a therapeutic amino acid sequence; and (b) one or more valent metal ions non-covalently attached to the polypeptide. A “tissue-targeting amino acid sequence” is defined herein to refer to an amino acid sequence that can bind or attach to tissue. A “diagnostic amino acid sequence” is an amino acid sequence that can be administered to a subject or contacted with a tissue for the purpose of facilitating diagnosis of a disease or disorder, or condition associated with abnormal cell physiology. A “therapeutic amino acid sequence” is defined herein to refer to an amino acid sequence that can be administered to a subject, or contacted with a cell or tissue, for the purpose of treating a disease or disorder, or preventing a disease or disorder, or treating or preventing an alteration or disruption of a normal physiologic process. For example, the therapeutic amino acid sequence may be an anti-cancer amino acid sequence, such as a chemotherapeutic agent. Chemotherapeutic agents are discussed elsewhere in this specification.

The one or more valent metal ions may be non-covalently attached to the tissue targeting amino acid sequence, the diagnostic amino acid sequence, and/or the therapeutic amino acid sequence, or the one or more valent metal ions may be non-covalently attached to the polypeptide at a separate amino acid sequence.

For example, in some embodiments, the valent metal ion is attached to a separate amino acid sequence that includes within its sequence one or more amino acids that function to bind valent metal ions. For example, this sequence may be a poly(glutamate) amino acid sequence or a poly(aspartate amino acid sequence). This amino acid sequence is fused or chemically conjugated with a tissue targeting amino acid sequence, a diagnostic amino acid sequence, or a therapeutic amino acid sequence to produce a chimeric polypeptide.

The chimeric polypeptides of the present invention may be produced by chemical synthetic methods or by chemical linkage between the two moieties. In certain particular embodiments, they are produced by fusion of a coding sequence of a valent metal ion-binding amino acid sequence and a coding sequence of tissue-targeting amino acid sequence, a diagnositic amino acid sequence, or a therapeutic amino acid sequence under the control of a regulatory sequence which directs the expression of the fusion polynucleotide in an appropriate host cell.

The fusion of two full-length coding sequences can be achieved by methods well known in the art of molecular biology. It is preferred that a fusion polynucleotide contain only the AUG translation initiation codon at the 5′ end of the first coding sequence without the initiation codon of the second coding sequence to avoid the production of two separate encoded products. In addition, a leader sequence may be placed at the 5′ end of the polynucleotide in order to target the expressed product to a specific site or compartment within a host cell to facilitate secretion or subsequent purification after gene expression. The two coding sequences can be fused directly without any linker or by using a flexible polylinker, such as one composed of the pentamer Gly-Gly-Gly-Gly-Ser (SEQ ID NO:1) repeated 1 to 3 times (see Huston et al., 1988, which is herein specifically incorporated by reference). Other linkers which may be used include Glu-Gly-Lys-Ser-Ser-Gly-Ser-Gly-Ser-Glu-Ser-Lys-Val-Asp (SEQ ID NO:2) (Chaudhary et al., 1990, herein specifically incorporated by reference) and Lys-Glu-Ser-Gly-Ser-Val-Ser-Ser-Glu-Gln-Leu-Ala-Gln-Phe-Arg-Ser-Leu-Asp (SEQ ID NO:3) (Bird et al., 1988, herein specifically incorporated by reference).

G. Imaging Modalities and Imaging Agents

Certain embodiments of the present invention pertain to methods of imaging a site within a subject that involves (a) administering to the subject a diagnostically effective amount of a composition comprising a valent metal ion—polypeptide chelate of the present invention, and (b) detecting a signal from the valent metal ion-polypeptide chelate that is localized at the site. Furthermore, as set forth above, in certain embodiments a second moiety that is an diagnostic/imaging moiety may be conjugated to the polypeptide-valent metal ion chelate. Any imaging modality known to those of ordinary skill in the art is contemplated as a means to detect a signal from the valent metal ion-polypeptide chelate or imaging moiety-polypeptide-valent metal ion complex that is localized at the site. Examples of imaging modalities are set forth as follows.

1. Examples of Imaging Modalities

a. Gamma Camera Imaging

A variety of nuclear medicine techniques for imaging are known to those of ordinary skill in the art. Any of these techniques can be applied in the context of the imaging methods of the present invention to measure a signal from the reporter. For example, gamma camera imaging is contemplated as a method of imaging that can be utilized for measuring a signal derived from the reporter. One of ordinary skill in the art would be familiar with techniques for application of gamma camera imaging see, e.g., Kundra et al., 2002, herein specifically incorporated by reference). In one embodiment, measuring a signal can involve use of gamma-camera imaging of a 111-In-octreotide-SSRT2A reporter system.

b. PET and SPECT

Radionuclide imaging modalities (positron emission tomography, {PET}; single photon emission computed tomography (SPECT) are diagnostic cross-sectional imaging techniques that map the location and concentration of radionuclide-labeled radiotracers. Although CT and MRI provide considerable anatomic information about the location and the extent of tumors, these imaging modalities cannot adequately differentiate invasive lesions from edema, radiation necrosis, grading or gliosis. PET and SPECT can be used to localize and characterize tumors by measuring metabolic activity.

PET and SPECT provide information pertaining to information at the cellular level, such as cellular viability. In PET, a patient ingests or is injected with a slightly radioactive substance that emits positrons, which can be monitored as the substance moves through the body. In one common application, for instance, patients are given glucose with positron emitters attached, and their brains are monitored as they perform various tasks. Since the brain uses glucose as it works, a PET image shows where brain activity is high.

Closely related to PET is single-photon emission computed tomography, or SPECT. The major difference between the two is that instead of a positron-emitting substance, SPECT uses a radioactive tracer that emits high-energy photons. SPECT is valuable for diagnosing coronary artery disease, and already some 2.5 million SPECT heart studies are done in the United States each year.

PET radiopharmaceuticals for imaging are commonly labeled with positron-emitters such as ¹¹C, ¹³N, ¹⁵O, ¹⁸F, ⁸²Rb, ⁶²Cu, and ⁶⁸Ga. SPECT radiopharmaceuticals are commonly labeled with positron emitters such as ^{99m}Tc, ²⁰¹Tl, and ⁶⁷Ga. Regarding brain imaging, PET and SPECT radiopharmaceuticals are classified according to blood-brain-barrier permeability, cerebral perfusion and metabolism receptor-binding, and antigen-antibody binding (Saha et al., 1994). The blood-brain-barrier SPECT agents, such as ^{99m}TcO4-DTPA, ²⁰¹Tl, and [⁶⁷Ga]citrate are excluded by normal brain cells, but enter into tumor cells because of altered BBB. SPECT perfusion agents such as [¹²³I]IMP, [^{99m}Tc]HMPAO, [^{99m}Tc]ECD are lipophilic agents, and therefore diffuse into the normal brain. Important receptor-binding SPECT radiopharmaceuticals include [¹²³I]QNE, [¹²³I]IBZM, and [¹²³I]iomazenil. These tracers bind to specific receptors, and are of importance in the evaluation of receptor-related diseases.

C. Computerized Tomography (CT)

Computerized tomography (CT) is contemplated as an imaging modality in the context of the present invention. By taking a series of X-rays, sometimes more than a thousand, from various angles and then combining them with a computer, CT made it possible to build up a three-dimensional image of any part of the body. A computer is programmed to display two-dimensional slices from any angle and at any depth.

In CT, intravenous injection of a radiopaque contrast agent can assist in the identification and delineation of soft tissue masses when initial CT scans are not diagnostic. Similarly, contrast agents aid in assessing the vascularity of a soft tissue or bone lesion. For example, the use of contrast agents may aid the delineation of the relationship of a tumor and adjacent vascular structures.

CT contrast agents include, for example, iodinated contrast media. Examples of these agents include iothalamate, iohexol, diatrizoate, iopamidol, ethiodol, and iopanoate. Gadolinium agents have also been reported to be of use as a CT contrast agent (see, e.g., Henson et al., 2004). For example, gadopentate agents has been used as a CT contrast agent (discussed in Strunk and Schild, 2004).

d. Magnetic Resonance Imaging (MRI)

Magnetic resonance imaging (MRI) is an imaging modality that is newer than CT that uses a high-strength magnet and radio-frequency signals to produce images. The most abundant molecular species in biological tissues is water. It is the quantum mechanical “spin” of the water proton nuclei that ultimately gives rise to the signal in imaging experiments. In MRI, the sample to be imaged is placed in a strong static magnetic field (1-12 Tesla) and the spins are excited with a pulse of radio frequency (RF) radiation to produce a net magnetization in the sample. Various magnetic field gradients and other RF pulses then act on the spins to code spatial information into the recorded signals. By collecting and analyzing these signals, it is possible to compute a three-dimensional image which, like a CT image, is normally displayed in two-dimensional slices.

Contrast agents used in MR imaging differ from those used in other imaging techniques. Their purpose is to aid in distinguishing between tissue components with identical signal characteristics and to shorten the relaxation times (which will produce a stronger signal on T1-weighted spin-echo MR images and a less intense signal on T2-weighted images). Examples of MRI contrast agents include gadolinium chelates, manganese chelates, chromium chelates, and iron particles.

Both CT and MRI provide anatomical information that aid in distinguishing tissue boundaries and vascular structure. Compared to CT, the disadvantages of MRI include lower patient tolerance, contraindications in pacemakers and certain other implanted metallic devices, and artifacts related to multiple causes, not the least of which is motion (Alberico et al., 2004). CT, on the other hand, is fast, well tolerated, and readily available but has lower contrast resolution than MRI and requires iodinated contrast and ionizing radiation (Alberico et al., 2004). A disadvantage of both CT and MRI is that neither imaging modality provides functional information at the cellular level. For example, neither modality provides information regarding cellular viability.

e. Optical Imaging

Optical imaging is another imaging modality that has gained widespread acceptance in particular areas of medicine. Examples include optical labelling of cellular components, and angiography such as fluorescein angiography and indocyanine green angiography. Examples of optical imaging agents include, for example, fluorescein, a fluorescein derivative, indocyanine green, Oregon green, a derivative of Oregon green derivative, rhodamine green, a derivative of rhodamine green, an eosin, an erythrosin, Texas red, a derivative of Texas red, malachite green, nanogold sulfosuccinimidyl ester, cascade blue, a coumarin derivative, a naphthalene, a pyridyloxazole derivative, cascade yellow dye, dapoxyl dye.

f. Ultrasound

Another biomedical imaging modality that has gained widespread acceptance is ultrasound. Ultrasound imaging has been used noninvasively to provide realtime cross-sectional and even three-dimensional images of soft tissue structures and blood flow information in the body. High-frequency sound waves and a computer to create images of blood vessels, tissues, and organs.

Ultrasound imaging of blood flow can be limited by a number of factors such as size and depth of the blood vessel. Ultrasonic contrast agents, a relatively recent development, include perfluorine and perfluorine analogs, which are designed to overcome these limitations by helping to enhance grey-scale images and Doppler signals.

2. Procedure for Dual Imaging

Certain embodiments of the present invention pertain to methods of imaging a site within a subject using two imaging modalities that involve measuring a first signal and a second signal from the imaging moiety-polypeptide-valent metal ion complex. The first signal is derived from the valent metal ion and the second signal is derived from the imaging moiety. As set forth above, any imaging modality known to those of ordinary skill in the art can be applied in these embodiments of the present imaging methods.

The imaging modalities are performed at any time during or after administration of the composition comprising the diagnostically effective amount of the composition of the present invention. For example, the imaging studies may be performed during administration of the dual imaging composition of the present invention, or at any time thereafter. In some embodiments, the first imaging modality is performed beginning concurrently with the administration of the dual imaging agent, or about 1 sec, 1 hour, 1 day, or any longer period of time following administration of the dual imaging agent, or at any time in between any of these stated times.

The second imaging modality may be performed concurrently with the first imaging modality, or at any time following the first imaging modality. For example, the second imaging modality may be performed about 1 sec, about 1 hour, about 1 day, or any longer period of time following completion of the first imaging modality, or at any time in between any of these stated times. In certain embodiments of the present invention, the first and second imaging modalities are performed concurrently such that they begin at the same time following administration of the One of ordinary skill in the art would be familiar with performance of the various imaging modalities contemplated by the present invention.

In some embodiments of the present methods of dual imaging, the same imaging device is used to perform a first imaging modality and a second imaging modality. In other embodiments, a different imaging device is used to perform the second imaging modality. One of ordinary skill in the art would be familiar with the imaging devices that are available for performance of a first imaging modality and a second imaging modality, and the skilled artisan would be familiar with use of these devices to generate images.

H. Radiolabeled Agents

As set forth above, certain embodiments of the compositions of the present invention include a valent metal ion chelated to a polypeptide as set forth above, wherein the valent metal ion is a radionuclide. Radiolabeled agents, compounds, and compositions provided by the present invention are provided having a suitable amount of radioactivity. For example, in forming ^{99m}Tc radioactive complexes, it is generally preferred to form radioactive complexes in solutions containing radioactivity at concentrations of from about 0.01 millicurie (mCi) to about 300 mCi per mL.

Radiolabeled imaging agents provided by the present invention can be used for visualizing sites in a mammalian body. In accordance with this invention, the imaging agents are administered by any method known to those of ordinary skill in the art. For example, administration may be in a single unit injectable dose. Any of the common carriers known to those with skill in the art, such as sterile saline solution or plasma, may be utilized after radiolabeling for preparing the compounds of the present invention for injection. Generally, a unit dose to be administered has a radioactivity of about 0.01 mCi to about 300 mCi, preferably 10 mCi to about 200 mCi. The solution to be injected at unit dosage is from about 0.01 mL to about 10 mL.

After intravenous administration of a diagnostically effective amount of a composition of the present invention, imaging can be performed. Imaging of a site within a subject, such as an organ or tumor can take place, if desired, in hours or even longer, after the radiolabeled reagent is introduced into a patient. In most instances, a sufficient amount of the administered dose will accumulate in the area to be imaged within about 0.1 of an hour. As set forth above, imaging may be performed using any method known to those of ordinary skill in the art. Examples include PET, SPECT, and gamma scintigraphy. In gamma scintigraphy, the radiolabel is a gamma-radiation emitting radionuclide and the radiotracer is located using a gamma-radiation detecting camera (this process is often referred to as gamma scintigraphy). The imaged site is detectable because the radiotracer is chosen either to localize at a pathological site (termed positive contrast) or, alternatively, the radiotracer is chosen specifically not to localize at such pathological sites (termed negative contrast).

I. Kits

Certain embodiments of the present invention are generally concerned with kits for preparing an imaging agent, wherein the kit includes a sealed container including a predetermined quantity of a polypeptide comprising two or more consecutive amino acids that will function to non-covalently bind valent metal ions; and a sufficient amount of a reducing agent to chelate a valent metal ion to at least one of the two aforementioned consecutive amino acids. In other embodiments, the kit for preparing an imaging agent includes a sealed container including a predetermined quantity of a polypeptide that includes within its sequence a tissue targeting amino acid sequence, a diagnostic amino acid sequence, and/or a therapeutic amino acid sequence, and a sufficient amount of a reducing agent to attach one or more valent metal ions to the polypeptide.

The kits of the present invention include a sealed vial containing a predetermined quantity of a polypeptide of the present invention and a sufficient amount of reducing agent to label the compound with a valent metal ion. In some embodiments of the present invention, the kit includes a valent metal ion that is a radionuclide. In certain further embodiments, the radionuclide is ^{99m}Tc. In further embodiments of the present invention, the polypeptide is labeled with a second moiety that is a diagnostic moiety, an imaging moiety, or a therapeutic moiety.

The kit may also contain conventional pharmaceutical adjunct materials such as, for example, pharmaceutically acceptable salts to adjust the osmotic pressure, buffers, preservatives, and the like.

In certain embodiments, an antioxidant included in the composition to prevent oxidation of the chelator moiety. In certain embodiments, the antioxidant is vitamin C (ascorbic acid). However, it is contemplated that any other antioxidant known to those of ordinary skill in the art, such as tocopherol, pyridoxine, thiamine, or rutin, may also be used. The components of the kit may be in liquid, frozen or dry form. In a preferred embodiment, kit components are provided in lyophilized form.

The cold instant kit is considered to be a commercial product. The cold instant kit could serve a radiodiagnostic purpose by adding pertechnetate. The technology is known as the “shake and shoot” method. The preparation time of radiopharmaceuticals would be less than 15 min. The same kit could also be chelated with different metals for different imaging applications. For instance, copper-61 (3.3 hrs half life) for PET; gadolinium for MRI. The cold kit itself could be used as a prodrug to treat disease. For example, the kit could be applied in tissue-specific targeted imaging and therapy.

J. Methods of Determining the Effectiveness of a Candidate Substance as an Imaging Agent

The present invention further contemplates methods of determining the effectiveness of a candidate substance as an imaging agent, involving: (a) obtaining a candidate substance; (b) conjugating or chelating the candidate substance to a polypeptide comprising within its sequence two or more consecutive amino acids that will function to bind valent metal ions; (c) introducing the candidate substance-polypeptide conjugate to a subject; and (d) detecting a signal from the candidate substance-polypeptide conjugate to determine the effectiveness of the candidate substance as an imaging agent.

These methods may comprise random screening of large libraries of candidate substances; alternatively, the assays may be used to focus on particular classes of candidate substances selected with an eye towards structural attributes that are believed to make them more likely to function as an imaging agent.

By function, it is meant that one may assay for the ability of a signal derived from the candidate substance to be measured following administration of the aforementioned candidate substance-polypeptide conjugate to a subject.

To identify the effectiveness of a candidate substance as an imaging agent, one generally will determine the ability to measure a signal from the polypeptide in the presence and absence of the candidate substance

Any imaging modality known to those of ordinary skill in the art, such as those imaging modalities set forth above, can be applied in the measurement of a signal from the candidate substance-polypeptpide conjugate.

It will, of course, be understood that all the screening methods of the present invention are useful in themselves notwithstanding the fact that effective imaging agents may not be found. The invention provides methods for screening for such candidates, not solely methods of finding them.

1. Modulators

As used herein the term “candidate substance” refers to any molecule that may potentially have activity as an imaging agent. The candidate substance may be a protein or fragment thereof, a small molecule, or even a nucleic acid molecule. It may prove to be the case that the most useful pharmacological compounds will be compounds that are structurally related to known imaging agents. Using lead compounds to help develop improved compounds is know as “rational drug design” and includes not only comparisons with know inhibitors and activators, but predictions relating to the structure of target molecules.

The goal of rational drug design is to produce structural analogs of known imaging agents. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for caniddate substances in an effort to “brute force” the identification of useful compounds. Combinatorial approaches also lend themselves to rapid evolution of potential imaging agents by the creation of second, third and fourth generation compounds modeled of active, but otherwise undesirable compounds.

Candidate substances may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Treatment of subjects, such as animals, with test compounds will involve the administration of the compound, in an appropriate form, to the animal. Administration will be by any route that could be utilized for clinical or non-clinical purposes, such as intravenously, by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

K. Hyperproliferative Disease

Certain aspects of the present invention pertain to compositions wherein a therapeutic moiety is conjugated to the polypeptide-valent metal ion chelate of the present invention. Thus, the composition of the present invention may, in certain embodiments, be useful in dual imaging and therapy. In certain particular embodiments, the therapeutic moiety is a moiety that is an agent known or suspected to be of benefit in the treatment or prevention of hyperproliferative disease in a subject. The subject may be an animal, such as a mammal. In certain particular embodiments, the subject is a human.

In other embodiments of the present invention, the valent metal ion is a therapeutic valent metal ion (e.g., Re-188, Re-186, Ho-166, Y-90, Sr-89, and Sm-153), and the polypeptide-valent metal ion chelate is an agent that is a therapeutic agent (rather than an imaging agent), that can be applied in the treatment or prevention of a hyperproliferative disease.

A hyperproliferative disease is herein defined as any disease associated with abnormal cell growth or abnormal cell turnover For example, the hyperproliferative disease may be cancer. The term “cancer” as used herein is defined as an uncontrolled and progressive growth of cells in a tissue. A skilled artisan is aware other synonymous terms exist, such as neoplasm or malignancy or tumor. Any type of cancer is contemplated for treatment by the methods of the present invention. For example, the cancer may be breast cancer, lung cancer, ovarian cancer, brain cancer, liver cancer, cervical cancer, colon cancer, renal cancer, skin cancer, head and neck cancer, bone cancer, esophageal cancer, bladder cancer, uterine cancer, stomach cancer, pancreatic cancer, testicular cancer, lymphoma, or leukemia. In other embodiments of the present invention, the cancer is metastatic cancer.

L. Dual Chemotherapy and Radiation Therapy (“Radiochemotherapy”)

In certain embodiments of the present invention, the compositions of the present invention are suitable for dual chemotherapy and radiation therapy (radiochemotherapy). For example, the polypeptide as set forth herein may be chelated to a valent metal ion that is a therapeutic valent metal ion, as well as a second moiety that is a therapeutic moiety (such as an anticancer moiety).

For example, the valent metal ion may be a beta-emitter. As herein defined, a beta emitter is any agent that emits beta energy of any range. Examples of beta emitters include Re-188, Re-186, Ho-166, Y-90, and Sn-153. One of ordinary skill in the art would be familiar with these agents for use in the treatment of hyperproliferative disease, such as cancer.

One of ordinary skill in the art would be familiar with the design of chemotherapeutic protocols and radiation therapy protocols that can applied in the administration of the compounds of the present invention. As set forth below, these agents may be used in combination with other therapeutic modalities directed at treatment of a hyperproliferative disease, such as cancer. Furthermore, one of ordinary skill in the art would be familiar with selecting an appropriate dose for administration to the subject. The protocol may involve a single dose, or multiple doses. The patient would be monitored for toxicity and response to treatment using protocols familiar to those of ordinary skill in the art.

M. Pharmaceutical Preparations

Pharmaceutical compositions of the present invention comprise a therapeutically or diagnostically effective amount of a composition of the present invention. The phrases “pharmaceutical or pharmacologically acceptable” or “therapeutically effective” or “diagnostically effective” refers to molecular entities and compositions that do not produce an adverse, allergic or other untoward reaction when administered to an animal, such as, for example, a human, as appropriate. The preparation of therapeutically effective or diagnostically effective compositions will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “a composition comprising a therapeutically effective amount” or “a composition comprising a diagnostically effective amount” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. Except insofar as any conventional carrier is incompatible with the active ingredient, its use in the present compositions is contemplated.

The compositions of the present invention may comprise different types of carriers depending on whether it is to be administered in solid, liquid or aerosol form, and whether it need to be sterile for such routes of administration as injection. The compositions of the present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, injection, infusion, continuous infusion, localized perfusion bathing target cells directly, via a catheter, via a lavage, in lipid compositions (e.g., liposomes), or by other method or any combination of the forgoing as would be known to one of ordinary skill in the art.

The actual required amount of a composition of the present invention administered to a patient can be determined by physical and physiological factors such as body weight, severity of condition, the tissue to be imaged, the type of disease being treated, previous or concurrent imaging or therapeutic interventions, idiopathy of the patient, and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of the polypeptide-valent metal ion chelate. In other embodiments, the an active compound may comprise between about 2% to about 75% of the weight of the unit, or between about 25% to about 60%, for example, and any range derivable therein. In other non-limiting examples, a dose may also comprise from about 0.1 mg/kg/body weight to about 1000 mg/kg/body weight or any amount within this range, or any amount greater than 1000 mg/kg/body weight per administration.

In any case, the composition may comprise various antioxidants to retard oxidation of one or more component. Additionally, the prevention of the action of microorganisms can be brought about by preservatives such as various antibacterial and antifungal agents, including, but not limited to parabens (e.g., methylparabens, propylparabens), chlorobutanol, phenol, sorbic acid, thimerosal or combinations thereof.

The compositions of the present invention may be formulated in a free base, neutral or salt form. Pharmaceutically acceptable salts include the salts formed with the free carboxyl groups derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising, but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

Sterile injectable solutions may be prepared using techniques such as filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

N. Combinational Therapy

Certain aspects of the present invention pertain to compositions comprising a polypeptide that includes a second moiety that is a therapeutic moiety. In other embodiments, the polypeptide includes an amino acid sequence that is a therapeutic amino acid sequence.

These compositions can be applied in the treatment of diseases, such as cancer, along with another agent or therapy method, preferably another cancer treatment. Treatment with these compositions of the present invention may precede or follow the other therapy method by intervals ranging from minutes to weeks. In embodiments where another agent is administered, one would generally ensure that a significant period of time did not expire between the time of each delivery, such that the agents would still be able to exert an advantageously combined effect on the cell. For example, it is contemplated that one may administer two, three, four or more doses of one agent substantially simultaneously (i.e., within less than about a minute) with the compositions of the present invention. In other aspects, a therapeutic agent or method may be administered within about 1 minute to about 48 hours or more prior to and/or after administering a therapeutic amount of a composition of the present invention, or prior to and/or after any amount of time not set forth herein. In certain other embodiments, a composition of the present invention may be administered within of from about 1 day to about 21 days prior to and/or after administering another therapeutic modality, such as surgery or gene therapy. In some situations, it may be desirable to extend the time period for treatment significantly, however, where several weeks (e.g., about 1 to 8 weeks or more) lapse between the respective administrations.

Various combinations may be employed, the claimed agent for dual chemotherapy and radiation therapy is designated “A” and the secondary agent, which can be any other therapeutic agent or method, is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of the compositions of the present invention to a patient will follow general protocols for the administration of chemotherapeutics, taking into account the toxicity, if any, of these agents. It is expected that the treatment cycles would be repeated as necessary. It also is contemplated that various standard therapies, as well as surgical intervention, may be applied in combination with the described agent. These therapies include but are not limited to additional chemotherapy, additional radiotherapy, immunotherapy, gene therapy and surgery.

a. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

b. Radiotherapy

Other factors that cause DNA damage and have been used extensively include what are commonly known as y-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionucleotide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

d. Genes

In yet another embodiment, the secondary treatment is a gene therapy in which a therapeutic composition is administered before, after, or at the same time as the therapeutic agents of the present invention. Delivery of a therapeutic amount of a composition of the present invention in conjunction with a vector encoding a gene product will have a combined anti-hyperproliferative effect on target tissues.

e. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

### O. EXAMPLES

### Example 1

### ^{99m}Tc-GAP-Estradiol (EDL) for Estrogen Receptor-Positive Characterization

Synthesis of 3-Aminoethyl Estradiol

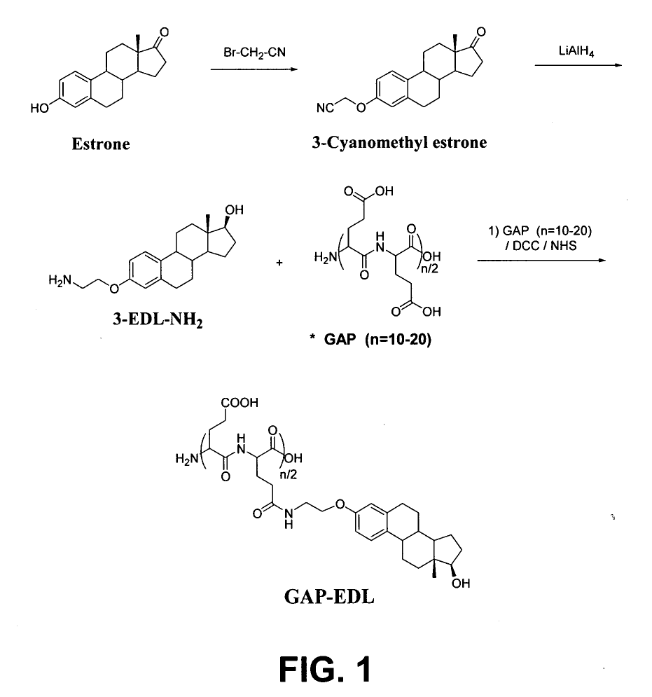

Estrone (1.47 g, 5.45 mmol) was dissolved in ethanol (50 ml). NaOEt (742 mg, 10.9 mmol) and bromoacetonitrile (0.5 ml, 1.722 g/ml, 6.65 mmol) were added. The reaction mixture was heated under reflux for 24 hrs. Ethanol was evaporated to dryness and ethyl acetate was added (100 ml). The mixture was washed with water (100 ml) in a separatory funnel. The organic layer was dried over magnesium sulfate and filtered. Ethyl acetate was evaporated under reduced pressure, and the solid product was washed with ether on filter paper. The yield of 3-acetonitrile estradiol was 75%. 3-Acetonitrile estradiol (620 mg, 2 mmol) was dissolved in THF (50 ml). Lithium aluminum hydride (IM in THF) was added and the reaction mixture was stirred overnight. The solvent was evaporated and the solid was dissolved in ethyl acetate and washed with water (100 ml) in a separatory funnel. The ethyl acetate layer was dried over magnesium sulfate and filtered. The solvent was evaporated. 3-Aminoethyl estradiol was collected with a yield of 92%. The synthetic schemes of ^{99m}Tc-GAP-EDL are shown in FIG. 1. The structure was confirmed by NMR spectrum.

Synthesis of ^{99m}Tc-Glutamate Peptide-Estradiol (GAP-EDL)

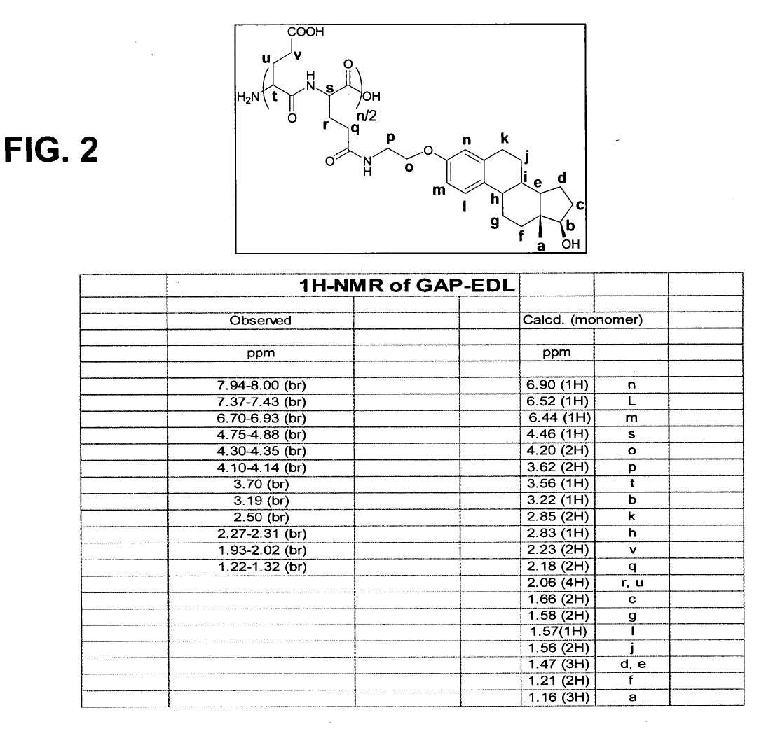

Sodium salt of glutamate peptide (GAP, 500 mg, MW 1,500-3,000) was converted to the acid form by adding 2 ml of 2N HCl, followed by dialysis for 48 hr using a Spectra/POR molecular porous membrane with a cut-off at 1000 (Spectrum Medical Industries Inc., Houston, Tex.). After freeze drying, the GAP acid (357.7 mg, 0.1589 mmole) was dissolved in DMF (10 ml). 3-Aminoethyl estradiol (502.5 mg, 1.59 mmole), dicyclohexyl carbodiimide (327.54 mg, 1.59 mmole) and 4-N,N-dimethylamino pyridine (194 mg, 1.59 mmole) were added. The mixture was stirred at room temperature for 2 days. After evaporation of DMF under high vacuum, the mixture was added with 2 ml of 1N sodium bicarbonate. The mixture was dialyzed with molecule weight cut-off at 1,000 for 48 hrs. The product was freeze dried, weighed 508 mg. The structure of GAP-EDL was confirmed by NMR spectrum (FIG. 2). There was 30% estradiol conjugated to GAP as determined by UV spectroscopy.

^{99m}Tc-pertechnetate (3.5 mCi) (Syncor Pharmaceutical Inc., Houston, Tex.) was added to a vial containing the lyophilized residue of GAP-EDL (5 mg) and tin chloride (II) (SnCl₂, 100 μg, 0.53 μmol) in 0.6 ml water. The product was purified by using a PD-10 column (sephadex G-25, 10 ml) (Sigma Chemical Company, St. Louis, Mo.) and eluted with PBS (5 ml). One ml of eluent was collected in each test tube. The product was isolated in Tubes 3-5, yielded 3.0 mCi (86%). Radiochemical purity was assessed by Radio-TLC scanner (Bioscan, Washington, D.C.) using IM ammonium acetate: methanol (4:1) as an eluant. The product was 97% pure.

In Vitro Cell Binding Affinity Studies

Five different cell lines were used for the assay. Three were breast cancer cell lines (13762NF, MCF7 and T47D) and two were ovarian cancer cell lines (sensitive and resistant to cisplatin). Briefly, cells (50,000/well) were treated with 20 μl of estrone (54 μg/well) or DMSO (control) and ^{99m}Tc-GAP-EDL (6 μg/well, 1 μCi/well). After 0.5-4 hrs incubation, the cells were washed twice with ice cold PBS (1 ml), and trypsin EDTA (0.1 ml) was added. After 2 min, PBS (0.4 ml) was added and the total volume containing cells was transferred to a test tube to count the activity. Each data represents an average of three measurements and that were calculated as percentage of uptake of ^{99m}Tc-GAP-EDL added.

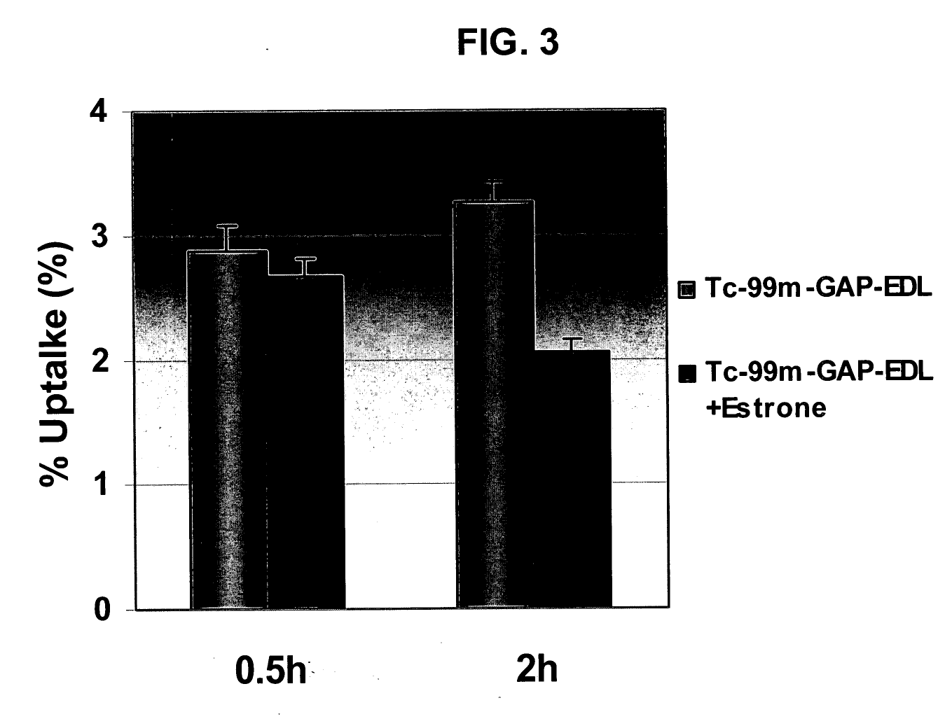

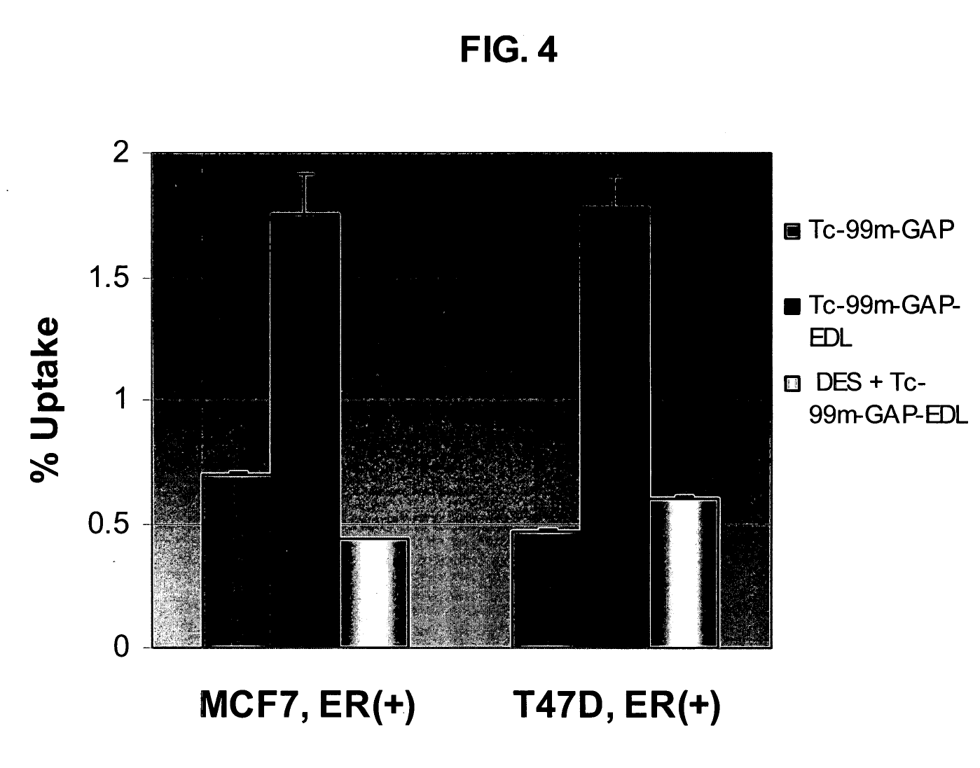

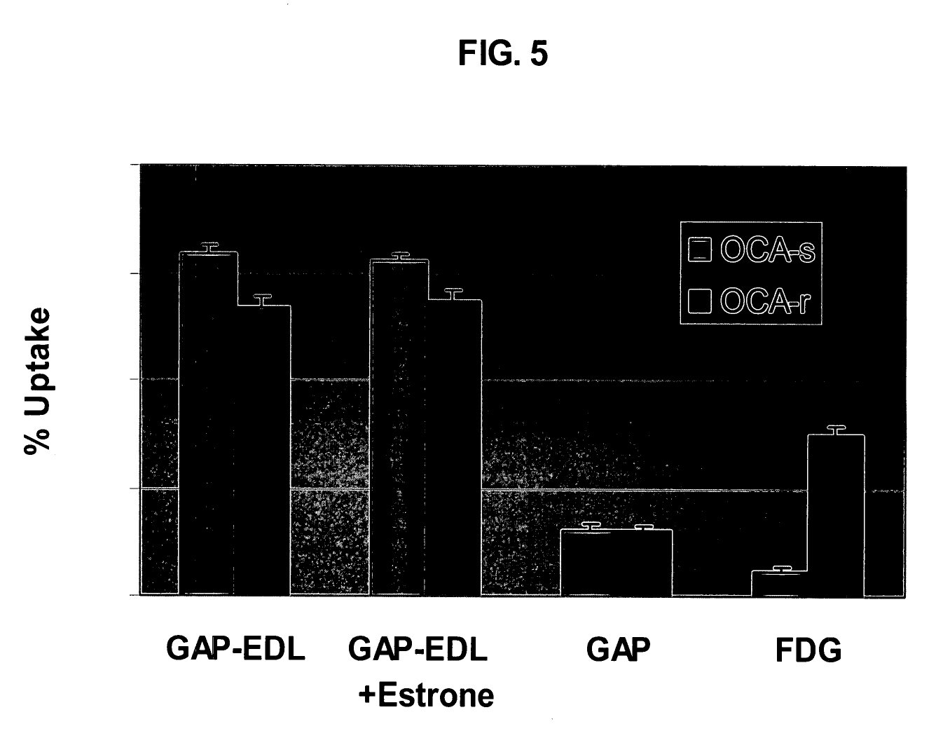

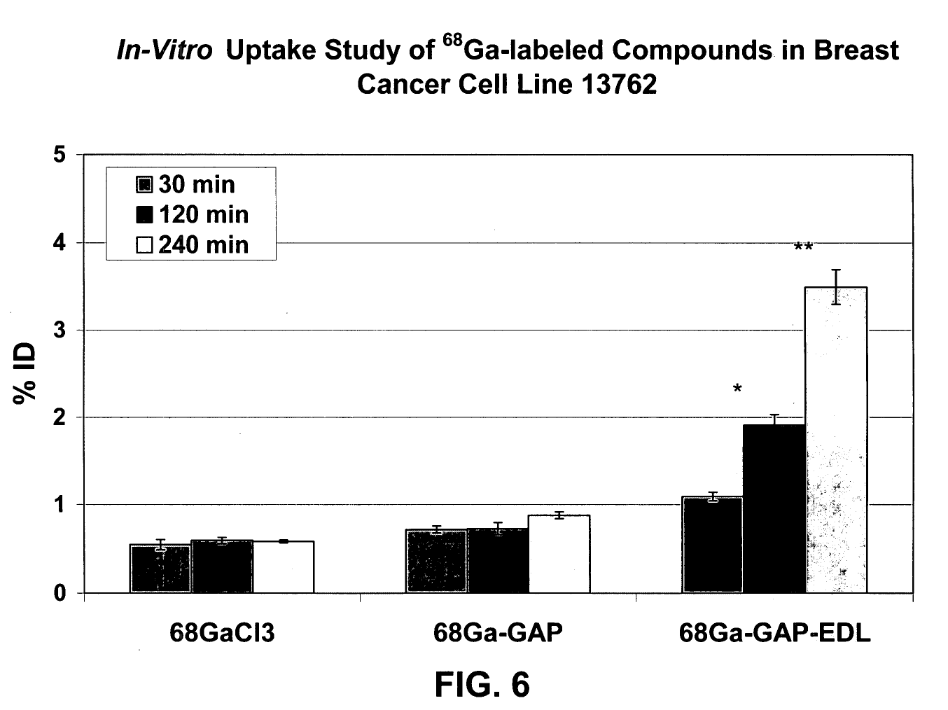

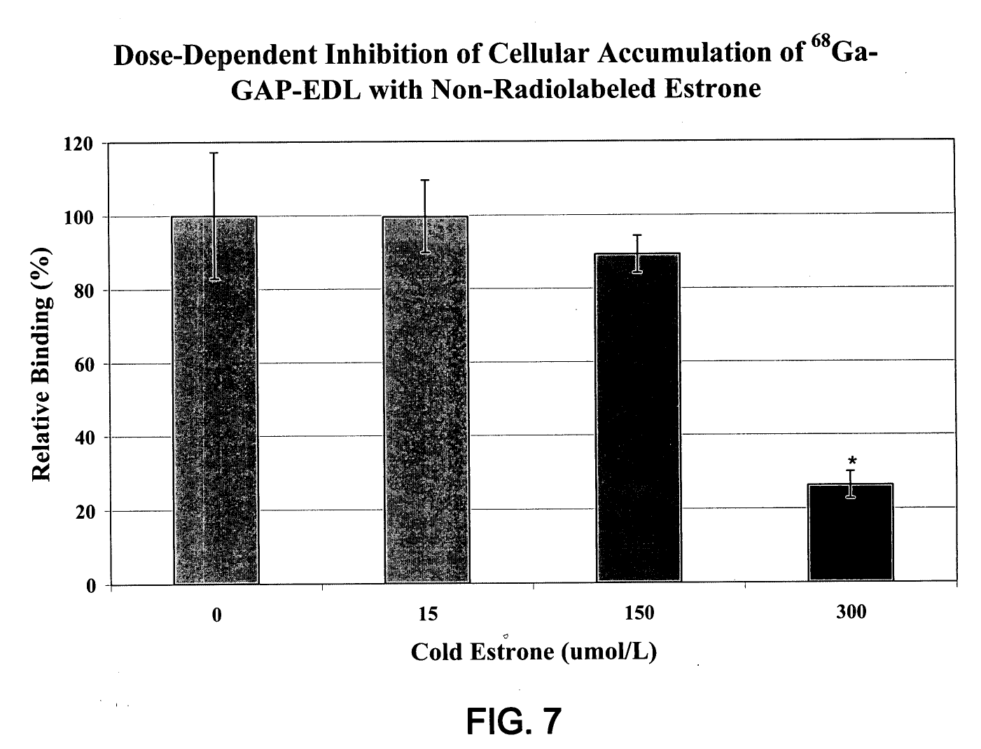

There was 10-40% decreased uptake in cells treated with estrone when compared to ^{99m}Tc-GAP-EDL (control) (FIG. 3). ^{99m}Tc-labeled GAP-estradiol conjugates could be blocked with estrone or diethylstilbestrol (FIG. 3 and FIG. 4). Using ER (−) ovarian cell lines, no marked difference of ^{99m}Tc-labeled estradiol uptake between cisplatin-sensitive and cisplatin-resistant ovarian tumor cells (FIG. 5). The findings suggest that cellular uptake of ^{99m}Tc-GAP-EDL is via an estrogen receptor-mediated process. Similar findings were observed with ⁶⁸Ga-GAP-EDL (FIG. 6 and FIG. 7).

Tissue Distribution Studies

Twelve female Fischer 344 rats (150±25 g) (Harlan Sprague-Dawley, Indianapolis, Ind.) (n=3 rats/time point) were inoculated (i.m.) with mammary tumor cells derived from the RBA CRL-1747 cell line. The cells were cultured in Eagle's MEM with Earle's BSS (90%) and fetal bovine serum (10%). Tumor cells (10⁶ cells/rat) were injected (i.m.) into the hind legs. Studies were performed 14 to 17 days after implantation when tumors were approximately 1 cm in diameter.

In tissue distribution studies, each animal was injected (i.v., 10 μCi/rat, 10 μg/rat) with ^{99m}Tc-GAP-EDL or ^{99m}Tc-GAP. Rats were sacrificed at 0.5-4 hrs. The selected tissues were excised, weighed and counted for radioactivity by using a gamma counter (Packard Instruments, Downers Grove, Ill.). The biodistribution of tracer in each sample was calculated as percentage of the injected dose per gram of tissue wet weight (% ID/g).

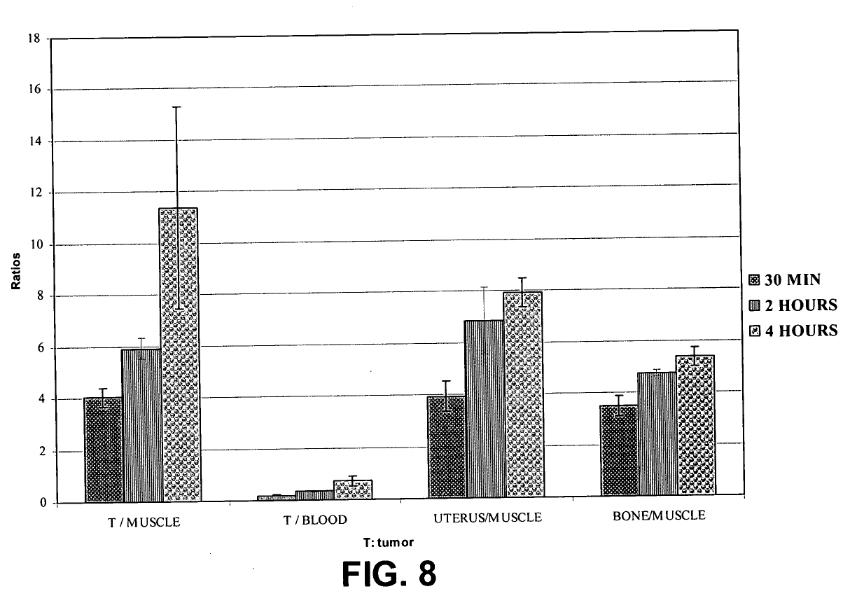

In vivo biodistribution studies showed that count density ratios for tumor-to-tissue and uterine-to-tissue were increased as a function of time in ^{99m}Tc-GAP-EDL groups (FIG. 8). At 4 hours, tumor-to-tissue and uterine-to-tissue count density ratios in ^{99m}Tc-GAP-EDL groups were significantly higher than in in ⁹⁹ mTc-GAP groups (Tables 3 and 4). The radiation dosimetry is shown in Table 5.

TABLE 3
Biodistribution of ^{99m}Tc-GAP in Breast Tumor-Bearing Rats
(MW. 750-3,000)
% of injected dose per gram of tissue weight (n = 3/time, interval, iv)

| | 30 MIN | 2 HOURS | 4 HOURS |
|---|---|---|---|
| BLOOD | 1.708 ± 0.050 | 0.924 ± 0.231 | 0.588 ± 0.019 |
| HEART | 0.473 ± 0.053 | 0.267 ± 0.057 | 0.180 ± .018 |
| LUNG | 0.852 ± 0.005 | 0.469 ± 0.114 | 0.333 ± 0.007 |
| LIVER | 3.454 ± 0.342 | 3.529 ± 0.333 | 2.876 ± 0.400 |
| SPLEEN | 1.628 ± 0.070 | 1.539 ± 0.390 | 1.072 ± 0.148 |
| KIDNEY | 10.141 ± 0.740 | 13.158 ± 4.090 | 11.702 ± 1.324 |
| INTESTINE | 0.285 ± 0.105 | 0.266 ± 0.041 | 0.151 ± 0.120 |
| UTERUS | 0.449 ± 0.016 | 0.388 ± 0.065 | 0.188 ± .038 |
| MUSCLE | 0.125 ± 0.016 | 0.066 ± 0.008 | 0.057 ± 0.025 |
| TUMOR | 0.520 ± 0.037 | 0.388 ± 0.038 | 0.323 ± .024 |
| THYROID | 0.653 ± 0.078 | 0.328 ± 0.111 | 0.339 ± 0.025 |
| STOMACH | 0.411 ± .032 | 0.301 ± 0.088 | 0.173 ± 0.017 |
| T/MUSCLE | 4.242 ± 0.881 | 5.981 ± 0.898 | 6.504 ± 2.908 |
| T/BLOOD | 0.304 ± 0.014 | 0.432 ± 0.082 | 0.549 ± 0.027 |
| UT/BLOOD | 0.263 ± 0.012 | 0.444 ± 0.154 | 0.321 ± 0.074 |
| UT/MUSCLE | 3.637 ± .466 | 6.009 ± 1.396 | 3.522 ± 0.802 |

Value shown represent the mean ± standard deviation of data from 3 animals

TABLE 4
Biodistribution of ^{99m}Tc-GAP-EDL in Breast Tumor-Bearing Rats
% of injected dose per gram of tissue weight (n = 3/time, interval, iv)

| | 30 min | 2 h | 4 h |
|---|---|---|---|
| BLOOD | 2.392 ± 0.022 | 1.226 ± 0.168 | 0.975 ± 0.044 |
| HEART | 0.516 ± 0.016 | 0.313 ± 0.044 | 0.308 ± 0.011 |
| LUNG | 1.059 ± 0.025 | 0.596 ± 0.081 | 0.479 ± 0.031 |
| LIVER | 6.191 ± 0.099 | 5.007 ± 0.758 | 5.331 ± 0.163 |
| SPLEEN | 2.253 ± 0.169 | 1.859 ± 0.250 | 2.139 ± 0.224 |
| KIDNEY | 8.079 ± 0.439 | 9.548 ± 1.255 | 12.312 ± 0.054 |
| INTESTINE | 0.428 ± 0.051 | 0.274 ± 0.048 | 0.275 ± 0.017 |
| UTERUS | 0.439 ± 0.062 | 0.455 ± 0.069 | 0.504 ± 0.020 |
| MUSCLE | 0.112 ± 0.002 | 0.068 ± 0.008 | 0.064 ± 0.002 |
| BONE | 0.391 ± 0.046 | 0.322 ± 0.030 | 0.344 ± 0.035 |
| TUMOR | 0.452 ± 0.041 | 0.408 ± 0.070 | 0.717 ± 0.233 |
| THYROID | 0.542 ± 0.046 | 0.334 ± 0.083 | 0.338 ± 0.015 |
| STOMACH | 0.359 ± 0.026 | 0.267 ± 0.034 | 0.206 ± 0.022 |
| T/MUSCLE | 4.037 ± 0.372 | 5.907 ± 0.407 | 11.377 ± 3.912 |
| T/BLOOD | 0.189 ± 0.016 | 0.330 ± 0.014 | 0.719 ± 0.202 |
| UTERUS/BLOOD | 0.184 ± 0.027 | 0.387 ± 0.140 | 0.518 ± 0.025 |
| UTERUS/MUSCLE | 3.929 ± 0.580 | 6.861 ± 1.298 | 7.923 ± 0.560 |
| BONE/MUSCLE | 3.484 ± 0.393 | 4.749 ± 0.123 | 5.363 ± 0.352 |

Values shown represent the mean ± standard deviation of data from 3 animals.

### MIRDOSE (IBM PC Version 3.1—August 1995)

TABLE 5
Radiation Dose Estimates for the REFERENCE ADULT
for ^{99m}TC-GAP-EDL

| | | TOTAL DOSE | | PRIMARY | | SECONDARY | |
|---|---|---|---|---|---|---|---|
| | TARGET ORGAN | mGy/MBq | rad/mCi | CONTRIBUTOR | % | CONTRIBUTOR | % |
| 1) | Adrenals | 8.24E−03 | 3.05E−02 | Liver | 72.8% | Kidneys | 23.2% |
| 2) | Brain | 1.51E−05 | 5.60E−05 | Liver | 74.2% | Lungs | 10.0% |
| 3) | Breasts | 1.08E−03 | 4.00E−03 | Liver | 86.9% | Kidneys | 4.9% |
| 4) | Gallbladder Wall | 1.32E−02 | 4.87E−02 | Liver | 91.0% | Kidneys | 8.2% |
| 5) | LLI Wall | 3.72E−04 | 1.38E−03 | Liver | 53.3% | Kidneys | 39.0% |
| 6) | Small Intestine | 2.23E−03 | 8.24E−03 | Liver | 72.0% | Kidneys | 25.2% |
| 7) | Stomach | 3.20E−03 | 1.18E−02 | Liver | 63.7% | Kidneys | 20.8% |
| 8) | ULI Wall | 3.22E−03 | 1.19E−02 | Liver | 80.5% | Kidneys | 17.4% |
| 9) | Heart Wall | 4.00E−03 | 1.48E−02 | Liver | 80.2% | Heart Conte | 9.7% |
| 10) | Kidneys | 4.08E−02 | 1.51E−01 | Kidneys | 89.1% | Liver | 9.9% |
| 11) | Liver | 4.55E−02 | 1.68E−01 | Liver | 98.1% | Kidneys | 1.7% |
| 12) | Lungs | 3.91E−03 | 1.45E−02 | Liver | 73.3% | Lungs | 18.8% |
| 13) | Muscle | 1.38E−03 | 5.10E−03 | Liver | 75.0% | Kidneys | 18.7% |
| 14) | Ovaries | 7.34E−04 | 2.72E−03 | Liver | 71.5% | Kidneys | 25.2% |
| 15) | Pancreas | 7.43E−03 | 2.75E−02 | Liver | 71.4% | Kidneys | 17.7% |
| 16) | Red Marrow | 1.68E−03 | 6.20E−03 | Liver | 68.3% | Kidneys | 26.9% |
| 17) | Bone Surfaces | 2.25E−03 | 8.32E−03 | Liver | 75.7% | Kidneys | 19.0% |
| 18) | Skin | 6.29E−04 | 2.33E−03 | Liver | 79.1% | Kidneys | 15.9% |
| 19) | Spleen | 1.66E−02 | 6.15E−02 | Spleen | 83.3% | Kidneys | 10.5% |
| 20) | Testes | 3.12E−05 | 1.16E−04 | Liver | 69.3% | Kidneys | 26.2% |
| 21) | Thymus | 1.00E−03 | 3.72E−03 | Liver | 81.4% | Heart Conte | 6.0% |
| 22) | Thyroid | 1.85E−03 | 6.86E−03 | Thyroid | 91.8% | Liver | 6.4% |
| 23) | Urin Bladder Wall | 2.15E−04 | 7.95E−04 | Liver | 74.6% | Kidneys | 23.0% |
| 24) | Uterus | 6.39E−04 | 2.36E−03 | Liver | 70.9% | Kidneys | 26.5% |
| 27) | Total Body | 2.78E−03 | 1.03E−02 | Liver | 80.0% | Kidneys | 15.2% |
| 28) | EFF DOSE EQUIV | 8.60E−03 | 3.18E−02 | Remainder | 86.7% | Lungs | 5.5% |
| 29) | EFF DOSE | 4.37E−03 | 1.62E−02 | Liver | 52.1% | Lungs | 10.7% |

Units of EDE and ED are mSv/MBq or rem/mCi.

RESIDENCE TIMES:

| | |
|---|---|
| Heart Contents | 1.90E−02 hr |
| Kidneys | 7.35E−01 hr |
| Liver | 3.83E+00 hr |
| Lungs | 5.50E−02 hr |
| Spleen | 1.65E−01 hr |
| Thyroid | 3.00E−03 hr |

MIRDOSE 3.1 Source Files:

| File Name | File Size (bytes) | Date and Time |
|---|---|---|
| MIRDOSE3.EXE | | |

Biodistribution comparison of GAP (G) vs GAP-DGAC (G-DG) with different molecular weight at 05, 2, 5, and 24 hrs.
Scintipraphic Imaging Studies

Scintigraphic images were obtained using a 2020 tc Imager gamma camera from Digirad (San Diego, Calif.) equipped with a low-energy parallel-hole collimator. The camera field of view is 20 cm×20 cm with an edge of 1.3 cm. The intrinsic spatial resolution is 3 mm and the matrix is 64×64. With a low-energy, high-resolution collimator installed, the system is designed for a planar sensitivity of at least 125 counts/minute (cpm)/μCi and spatial resolution of 7.6 mm.

Scintigraphic images were obtained at 0.5-4.0 hrs after i.v. injection of ^{99m}Tc-GAP-EDL and ^{99m}Tc-EDTA, respectively. To ascertain whether the tumor uptake by with ^{99m}Tc-GAP-EDL was related to estrogen receptors, we performed a blocking study. Each rat was pretreated with diethylstilbestrol (n=3, 10 mg/kg, iv) I hr prior to receiving ^{99m}Tc-GAP-EDL (300 μCi/rat, iv) and imaged at 0.5-4.0 hrs. Computer outlined regions of interest (ROI) (counts per pixel) were used to determine tumor-to-background count density ratios.

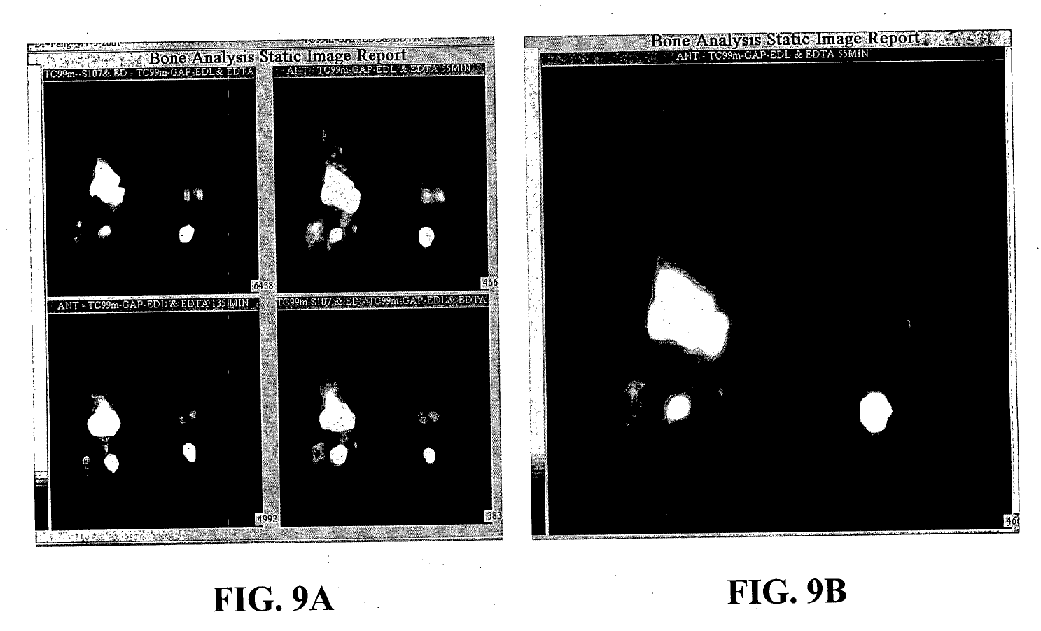

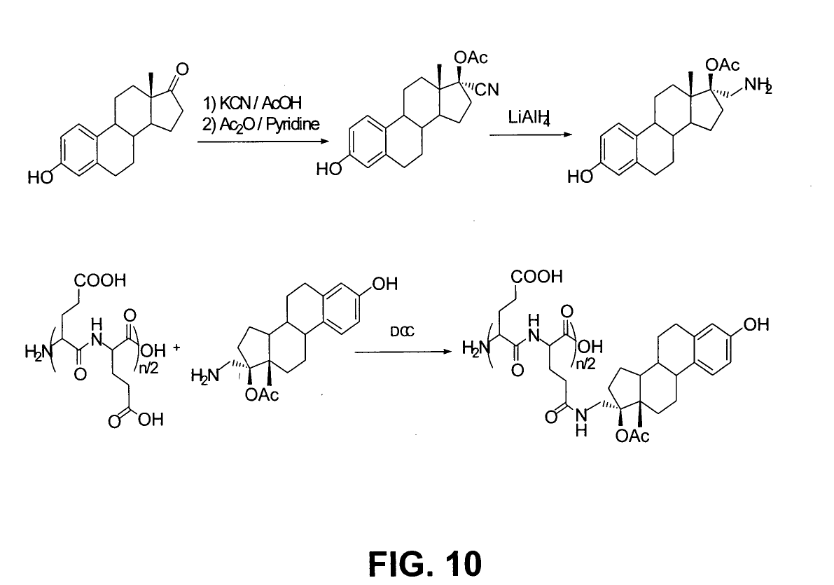

Tumor could be well visualized at 0.5-4 hrs (FIG. 9). ROI analysis of images at 0.5-4 hrs showed that tumor-to muscle ratios were 1.67-2.95 and 1.26-1.75 for ^{99m}Tc-GAP-EDL and ^{99m}Tc-EDTA, respectively. In blocking studies, tumor-to muscle ratios were 1.98-2.39 and 1.21-1.63 for ^{99m}Tc-GAP-EDL and blocked groups, respectively. There was a marked decrease in rats pretreated with diethylstilbestrol. The findings suggest tumor uptake of ^{99m}Tc-GAP-EDL is via an estrogen receptor-mediated process.

Synthesis of GAP-17-EDL

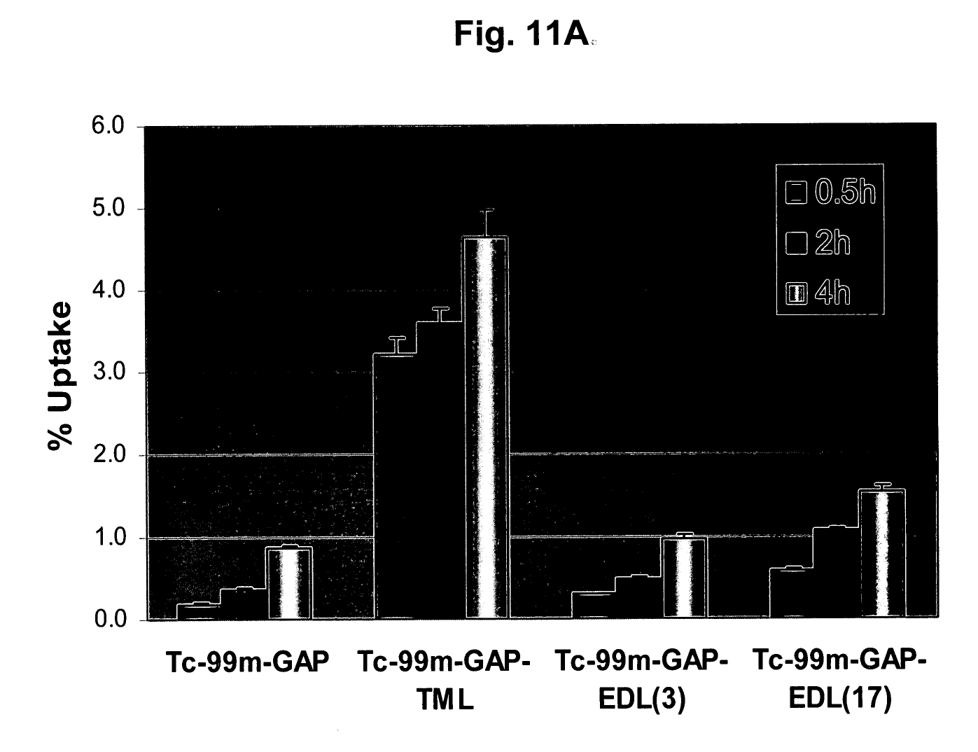

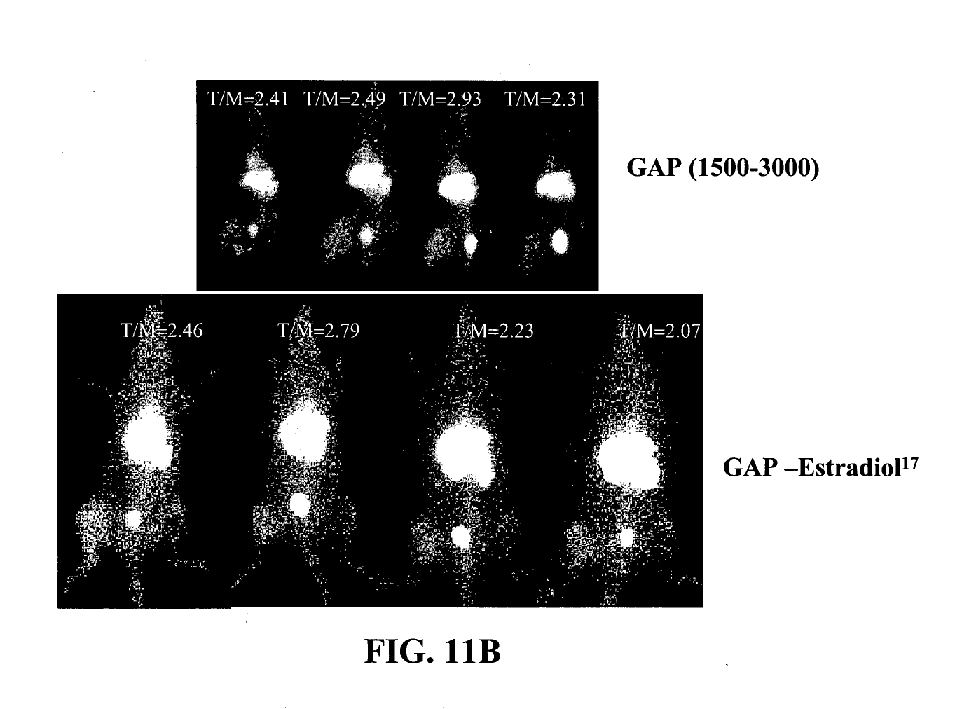

In addition to position 3 GAP-EDL, position 17 of estradiol was also conjugated to GAP. 17-EDL-NH₂ was prepared by reacting estrone with NaCN. The nitrile analogue was then reduced with LiAlH4 in tetrahydrofuran. 150 mg of GAP (Mol Wt 1500-3000) was dissolved in 5 ml of anhydrous DMF. 35.4 mg of DCC and 45 mg of 17-EDL-NH₂ were added. The mixture was stirred overnight at room temperature. 10 ml of water was added after removing solvent under reduced pressure. Water layer was filtered with 0.8 micrometer membrane. The filtrate was dialyzed with MW CO<1,000 membrane. 165 mg of white powder (GAP-17-EDL, Yield 85.8%) after drying with lyophilizer was obtained. The synthetic scheme is shown in FIG. 10. Proton NMR spectra of 17 EDL-NH₂ and GAP-EDL (position 17) confirmed the respective structures. The cellular uptake of this position 17 GAP-EDL is similar to position 3 GAP-EDL (FIG. 11A). The animal images are shown in FIG. 11B.

### EXAMPLE 2

### ^{99m}Tc-GAP-celebrex (COXi) for Cyclooxypenase-2 Characterization

Synthesis of COXi-OEt

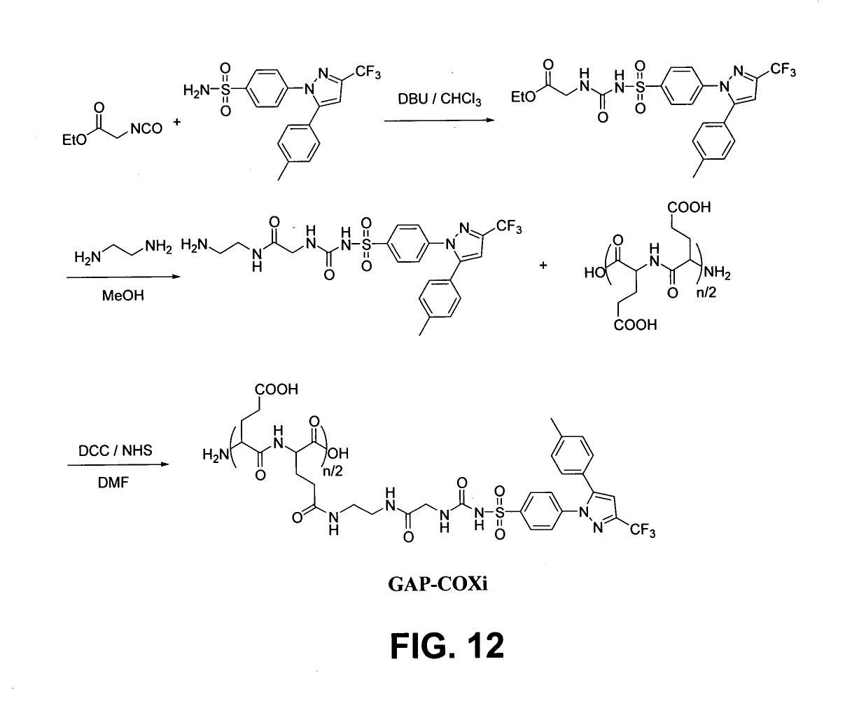

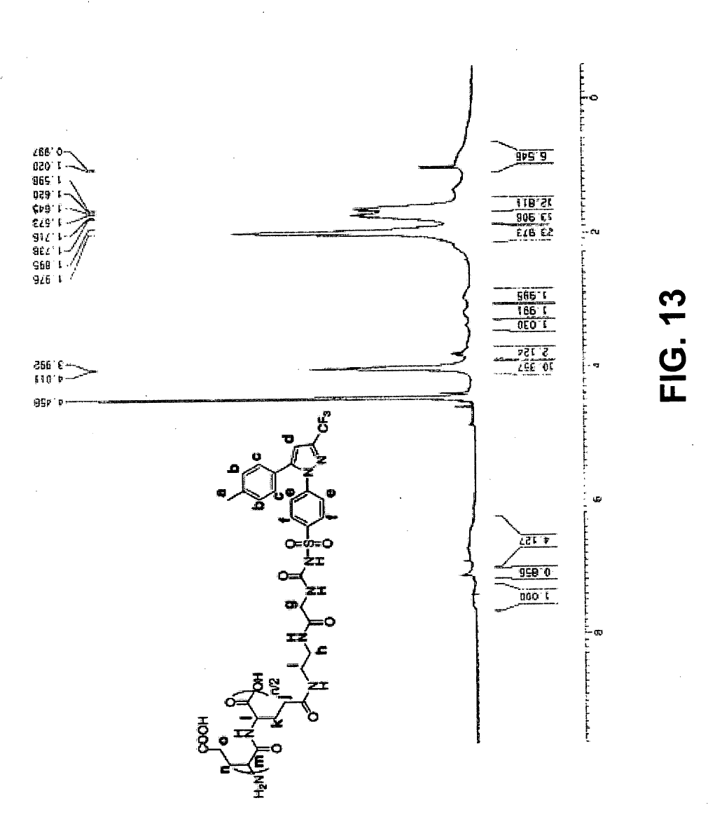

381.4 mg (1.0 mmol) of Celebrex (BZF, 4-[5-(4-methylphenyl)-3-(trifluoromethyl)-1-pyrazol-1-yl]-Benzenesulfonamide) and 152.4 mg (11.0 mmol) of DBU (1,8-diazabicyclo[5.4.0]undec-7-ene) were dissolved in 10 ml of chloroform. 129.1 mg (1.0 mmol) of ethyl isocyanatoacetate in 5 ml chloroform was then added dropwisely. The mixture was stirred for 3 hours at room temperature. The solvent was evaporated in vacuo and the crude product was loaded onto a silica gel packed column (mobile phase: chloroform and methanol gradient). The product (418.6 mg, white solid) was then isolated as (82% yield). Synthetic scheme of GAP-Coxi is shown in FIG. 12. Proton NMR of BZF and COXi-OEt confirmed the respective structures (FIG. 13).

Synthesis of COXi-NH₂

420.7 mg of ethylene diamine (7.0 mmol) was added into 357.4 mg (0.7 mmol) of COXi-OEt in 6 ml of ethanol. The mixture was stirred for 16 hours at room temperature. The solvent was then evaporated. The mixture was dissoolved in 10 ml chloroform and washed twice with 7 ml Brine. The chloroform layer was dried over magnesium sulfate anhydrous, filtered and evaporated. The crude reaction mixture 381 mg (crude yield 103.8%) was loaded onto a silica gel packed column and eluted with chloroform and methanol. COXi-NH₂ 304.7 mg (white solid, 83% yield) is obtained. Proton NMR and mass spectrometry confirmed the structure.

Synthesis of GAP-COXi

353 mg of GAP was dissolved in 15 ml of anhydrous DMF. 25.0 mg of DCC (dicyclohexylcarboimide) and 108 mg of COXi-NH₂ were added and stirred overnight at room temperature. The solvent was evaporated in vacuo and the crude product was added with 15 ml of water. 0.5N-sodium bicarbonate was then added to adjust pH to 8. The mixture was filtered with 0.8 micrometer membrane filter and dialyzed (MW CO<1,000 membrane). GAP-COXi (378 mg, whiter powder, yield 82.4%) after drying with lyophilizer was obtained. Proton NMR data of GAP-COXi confirmed the structure.

Tissue Distribution Studies

Twelve female Fischer 344 rats (150±25 g) (Harlan Sprague-Dawley, Indianapolis, Ind.) (n=3 rats/time point) were inoculated (i.m.) with mammary tumor cells derived from the RBA CRL-1747 cell line. The cells were cultured in Eagle's MEM with Earle's BSS (90%) and fetal bovine serum (10%). Tumor cells (10⁶ cells/rat) were injected (i.m.) into the hind legs. Studies were performed 14 to 17 days after implantation when tumors were approximately 1 cm in diameter.

In tissue distribution studies, each animal was injected (i.v., 10 μCi/rat, 10 μg/rat) with ^{99m}Tc-GAP-COXi or ^{99m}Tc-GAP. Rats were sacrificed at 0.5-4 hrs. The selected tissues were excised, weighed and counted for radioactivity by using a gamma counter (Packard Instruments, Downers Grove, Ill.). The biodistribution of tracer in each sample was calculated as percentage of the injected dose per gram of tissue wet weight (% ID/g).

In vivo biodistribution studies showed that count density ratios for tumor-to-muscle (blood) in ^{99m}Tc-GAP-COXi groups were increased as a function of time and higher value than ^{99m}Tc-GAP groups (Tables 3 and 6).

TABLE 6
Biodistribution of ^{99m}Tc-GAP-COX2i in Breast Tumor-Bearing Rats
% of injected dose per gram of tissue weight (n = 3/time, interval, iv)

| | 30 min | 2 h | 4 h |
|---|---|---|---|
| BLOOD | 1.097 ± 0.207 | 0.423 ± 0.060 | 0.246 ± 0.019 |
| HEART | 0.257 ± 0.040 | 0.126 ± 0.020 | 0.080 ± 0.006 |
| LUNG | 0.636 ± 0.084 | 0.367 ± 0.037 | 0.194 ± 0.014 |
| LIVER | 6.321 ± 0.169 | 6.661 ± 0.362 | 5.749 ± 0.552 |
| SPLEEN | 2.758 ± 0.352 | 2.816 ± 0.367 | 2.369 ± 0.447 |
| KIDNEY | 10.338 ± 0.491 | 12.659 ± 0.661 | 13.331 ± 1.246 |
| INTESTINE | 0.235 ± 0.013 | 0.179 ± 0.036 | 0.107 ± .014 |
| UTERUS | 0.389 ± .011 | 0.182 ± 0.022 | 0.141 ± 0.007 |
| MUSCLE | 0.070 ± 0.020 | 0.038 ± 0.006 | 0.020 ± 0.001 |
| TUMOR | 0.359 ± 0.031 | 0.267 ± 0.026 | 0.209 ± 0.004 |
| THYROID | 0.351 ± 0.071 | 0.162 ± 0.009 | 0.119 ± 0.013 |
| STOMACH | 0.252 ± 0.061 | 0.133 ± 0.028 | 0.079 ± .006 |
| BONE & | 0.347 ± 0.027 | 0.308 ± 0.012 | 0.289 ± .021 |
| JOINT | | | |
| TUMOR/ | 5.465 ± 1.150 | 7.148 ± 0.543 | 10.701 ± 0.166 |
| MUSCLE | | | |
| TUMOR/ | 0.334 ± 0.035 | 0.653 ± 0.109 | 0.858 ± 0.052 |
| BLOOD | | | |
| UTERUS/ | 0.365 ± 0.059 | 0.432 ± 0.012 | 0.583 ± 0.059 |
| BLOOD | | | |
| UTERUS/ | 6.022 ± 1.603 | 4.888 ± 0.607 | 7.268 ± 0.545 |
| MUSCLE | | | |
| BONE/ | 5.545 ± 2.001 | 8.533 ± 1.578 | 14.830 ± 1.334 |
| MUSCLE | | | |

Values shown represent the mean ± standard deviation of data from 3 animals.

Scintipraphic Imaging and Autoradiographic Studies

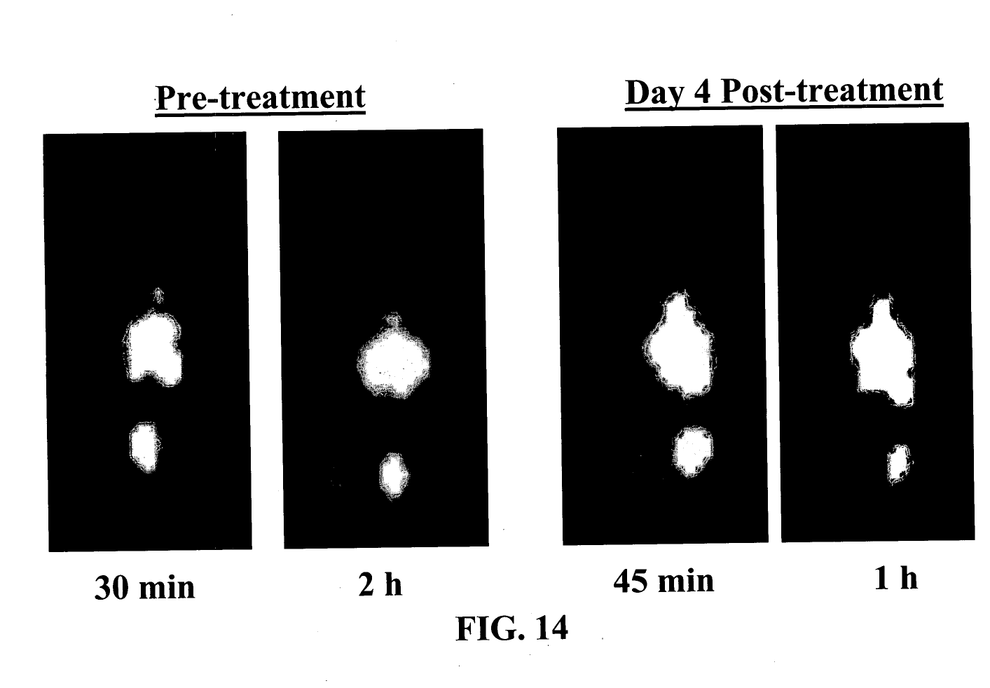

Scintigraphic images were obtained at 0.5-4.0 hrs after i.v. injection of ^{99m}Tc-GAP-COX2i (300 μCi/rat, iv) and imaged at 0.5-4.0 hrs in mammary tumor-bearing rats (derived from RBA CRL-1747 cell line). Whole-body autoradiograms were obtained by a quantitative image analyzer (Cyclone Storage Phosphor System, Packard, Meridian, Conn.). Following intravenous injection of ^{99m}Tc-GAP-COX2i (100 μCi/rat, iv), athymic nude mice bearing human uterine sarcoma in the right leg were killed at 1 hour and the body was fixed in carboxymethyl cellulose (3%). The frozen body was mounted onto a cryostat (LKB 2250 cryomicrotome) and cut into 100 μm coronal sections. Each section was thawed and mounted on a slide. The slide was then placed in contact with multipurpose phosphor storage screen (MP, 7001480) and exposed for 16 hrs. Tumor could be well visualized at 0.5-4 hrs (FIG. 14). Similar findings were observed in autoradiograms.

### EXAMPLE 3

### ^{99m}Tc-GAP-Doxorubicin (DOX) for Topoisomerase Characterization

Synthesis of GAP-DOX

112.5 mg of GAP (Mol Wt 1500-3000) was dissolved in 10 ml of anhydrous DMF. 51.6 mg of DCC (dicyclohexylcarboimide) and 33.8 mg of DOX (Doxorubicin hydrochloride) were added and stirred overnight at room temperature. The sovent was evaporated in vacuo. 5 ml of water and 0.7 ml of 1 N-sodium hydroxide solution was then added. pH value in water layer was 7.0. The mixture was filtered with 0.8 micrometer membrane filter and dialyzed with MW CO<1,000 membrane. 139 mg of red powder (GAP-DOX, Yield 79.1%) after drying with lyophilizer was obtained.

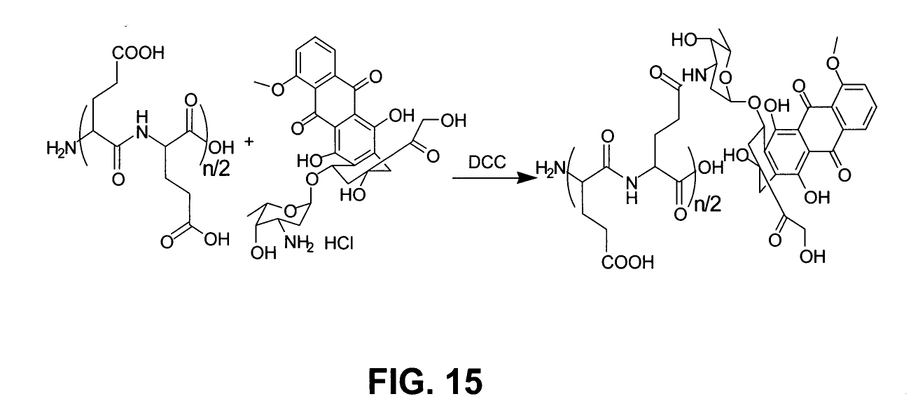

A standard curve was established from UV observance at 485-490 nm with various doxolubicin concentrations. Composition of GAP-DOX has 72% of GAP and 28% DOX. Synthetic scheme and proton NMR are shown in FIG. 15.

In Vitro Cell Binding Affinity Studies

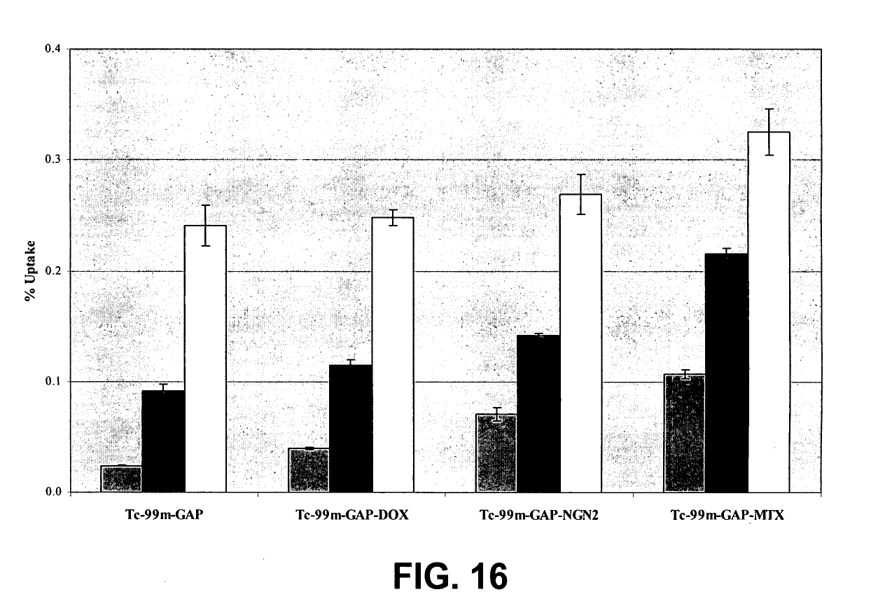

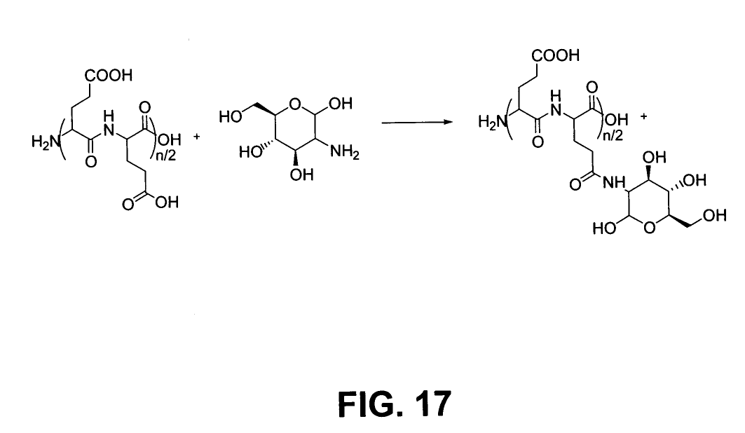

Two different cell lines were used for the assay—breast (13762NF) and lung cancer cell lines. Briefly, cells (50,000/well) were treated with 20 μl of ^{99m}Tc-GAP-DOX or GAP (control) (6 kg/well, 1 μCi/well). After 0.5-4 hrs incubation, the cells were washed twice with ice cold PBS (1 ml), and trypsin EDTA (0.1 ml) was added. After 2 min, PBS (0.4 ml) was added and the total volume containing cells was transferred to a test tube to count the activity. Each data represents an average of three measurements and that were calculated as percentage of uptake of ^{99m}Tc-GAP-DOX added. There was no marked difference of cellular uptake between ^{99m}Tc-labeled GAP and GAP-DOX (FIG. 16).

### EXAMPLE 4

### ^{99m}Tc-GAP-Sugar for Glycolytic Characterization

Synthesis of GAP-DG (mono sugar)

Wet Method: 225 mg of GAP (salt form, Mol Wt 1500-3000) was dissolved in 2 ml of water. 65.1 mg of S-NHS(1-hydroxy-2,5-dioxo-3-pyrrolidinesulfonic acid, monosodium salt hydrate) and 0.4 ml of 1N-sodium hydroxide solution were added with stirring. 21.6 mg of DG (glucosamine hydrochloride) and 57.5 mg of EDAC (1-(3-dimethylaminopropyl)-3-ethylcarbodiimide hydrochloride) were added and stirred overnight at room temperature. Reaction mixture was filtered with 0.8 micrometer membrane filter and dialyzed with MW CO<1,000 membrane. 215 mg of white powder (GAP-DG, Yield 89.2%) after drying with lyophilizer was obtained.

Dry Method: 225 mg of GAP (Mol Wt 1500-3000) was dissolved in 15 ml of anhydrous DMF. 62 mg of DCC and 21.6 mg of DG were added and stirred overnight at room temperature. 5 ml of water was added after removing solvent under reduced pressure. Water layer was filtered with 0.8 micrometer membrane filter and dialyzed with MW CO<1,000 membrane. 195 mg of white powder (GAP-DG, Yield 80.9%) after drying with lyophilizer was obtained. Synthetic scheme and proton NMR data of GAP-DG is shown in FIG. 17.

Synthesis of GAP-GAL (Mono Sugar)

Wet Method: 225 mg of GAP (salt form, Mol Wt 1500-3000) was dissolved in 2 ml of water. 65.1 mg of S—NHS(1-hydroxy-2,5-dioxo-3-pyrrolidinesulfonic acid, monosodium salt hydrate) and 0.4 ml of 1N-sodium hydroxide solution were added with stirring. 21.6 mg of GAL (Galactosamine hydrochloride) and 57.5 mg of EDAC (1-(3-dimethylaminopropyl)-3-ethylcarbodiimide hydrochloride) were added and stirred overnight at room temperature. Reaction mixture was filtered with 0.8 micrometer membrane filter and dialyzed with MW CO<1,000 membrane. 165.7 mg of white powder (GAP-GAL, Yield 68.7%) after drying with lyophilizer was obtained.

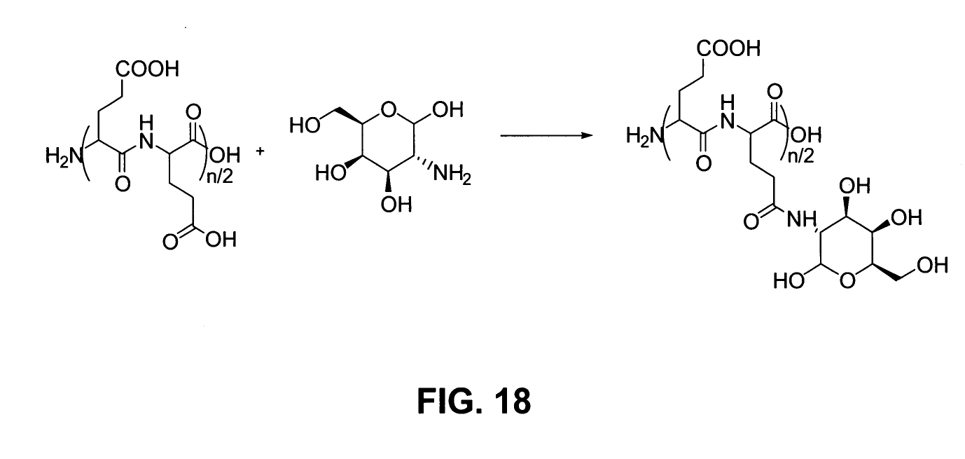

Dry Method: 225 mg of GAP (Mol Wt 1500-3000) was put into 15 ml of anhydrous DMF. 68.9 mg of DCC and 60 mg of GAL were added and stirred overnight at room temperature. 10 ml of water was added after removing solvent under reduced pressure. Water layer was filtered with 0.8 micrometer membrane filter and dialyzed with MW CO<1,000 membrane. 174.6 mg of white powder (GAP-GAL, Yield 71.3%) after drying with lyophilizer was obtained. Synthetic scheme and proton NMR data of GAP-GAL is shown in FIG. 18.

Synthesis of GAP-DGAc (Mono Sugar)

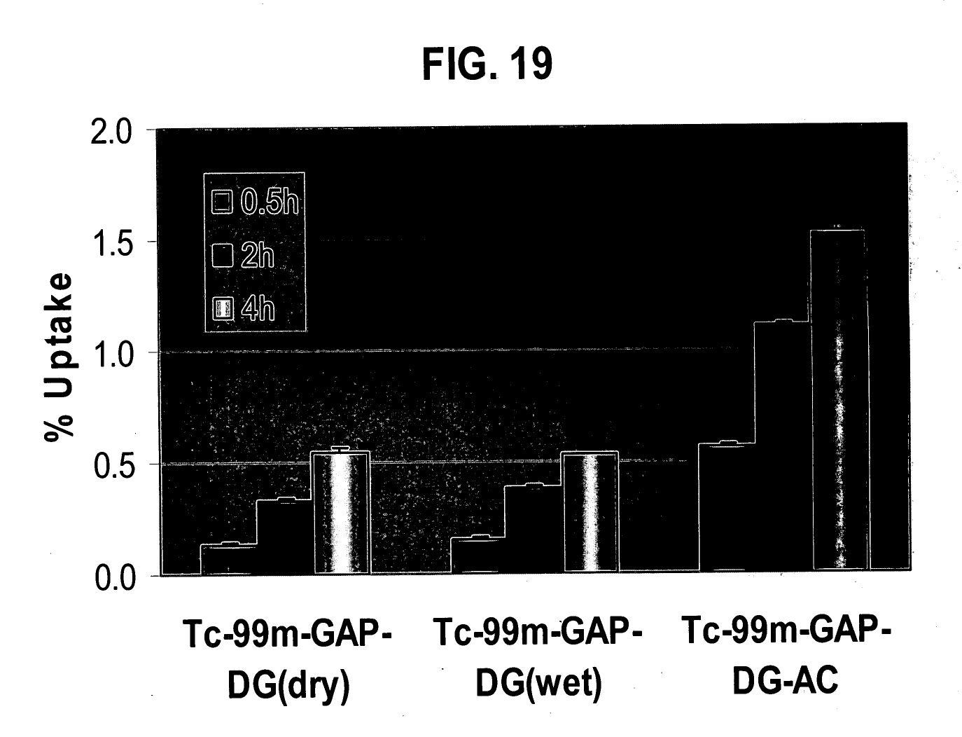

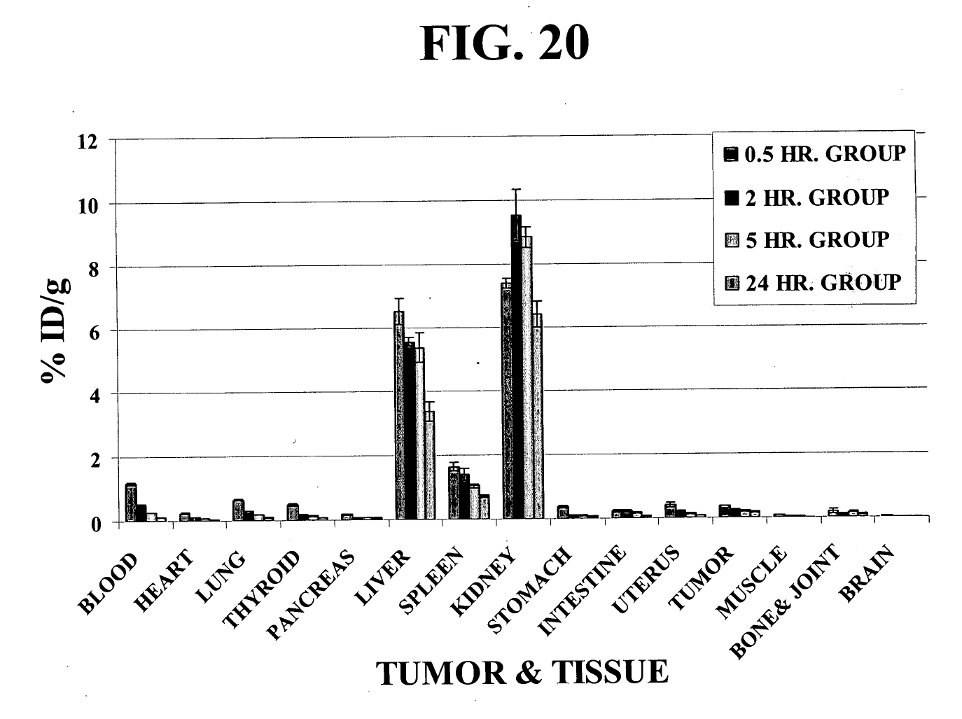

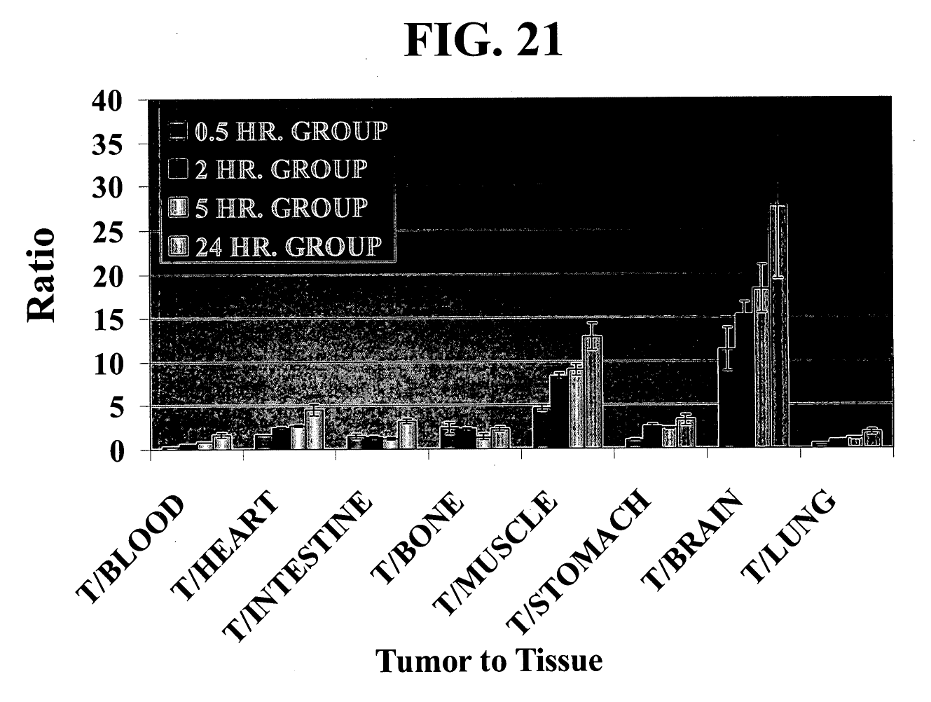

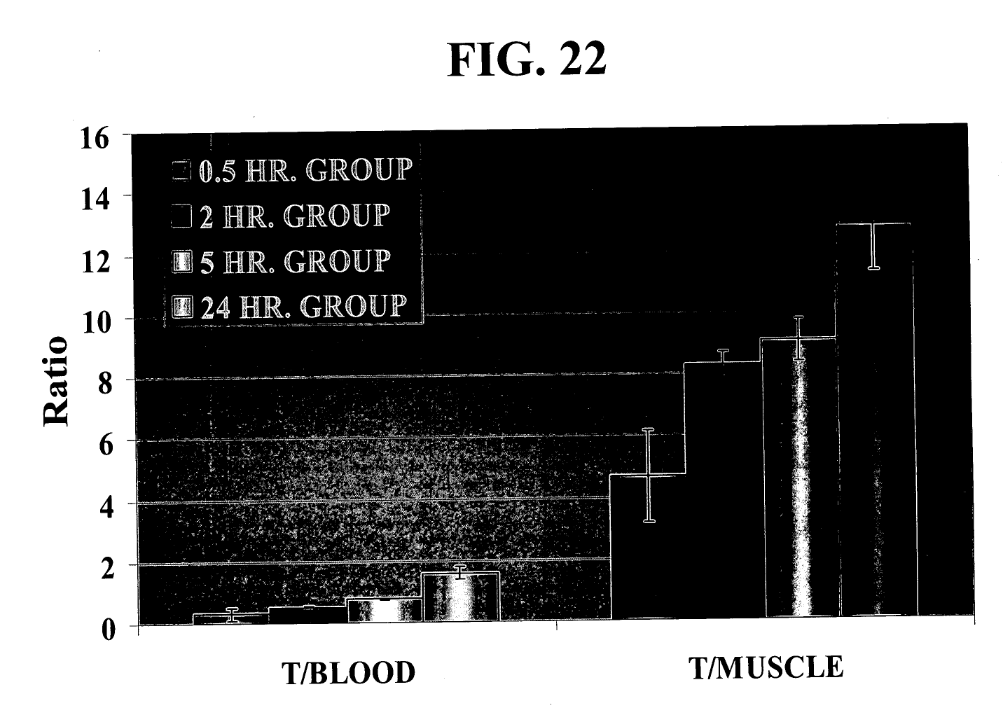

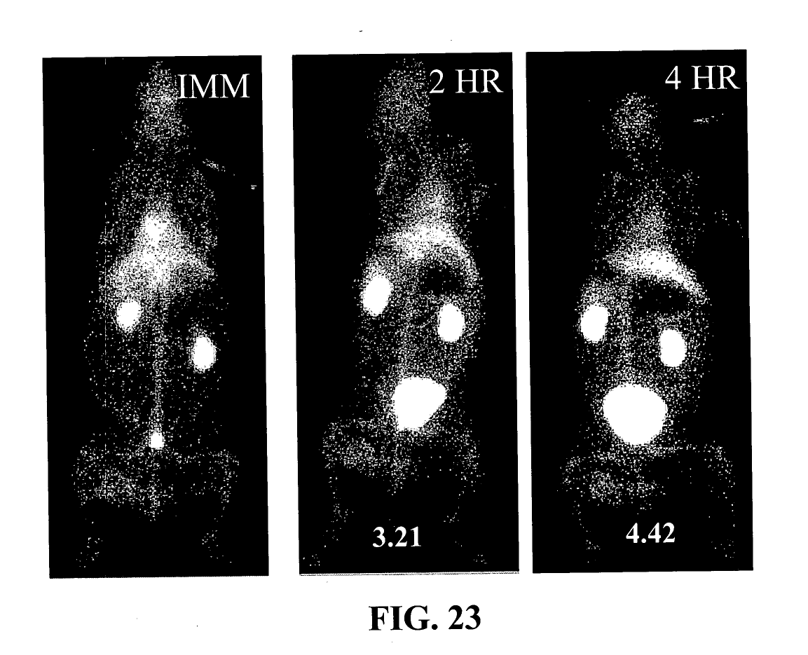

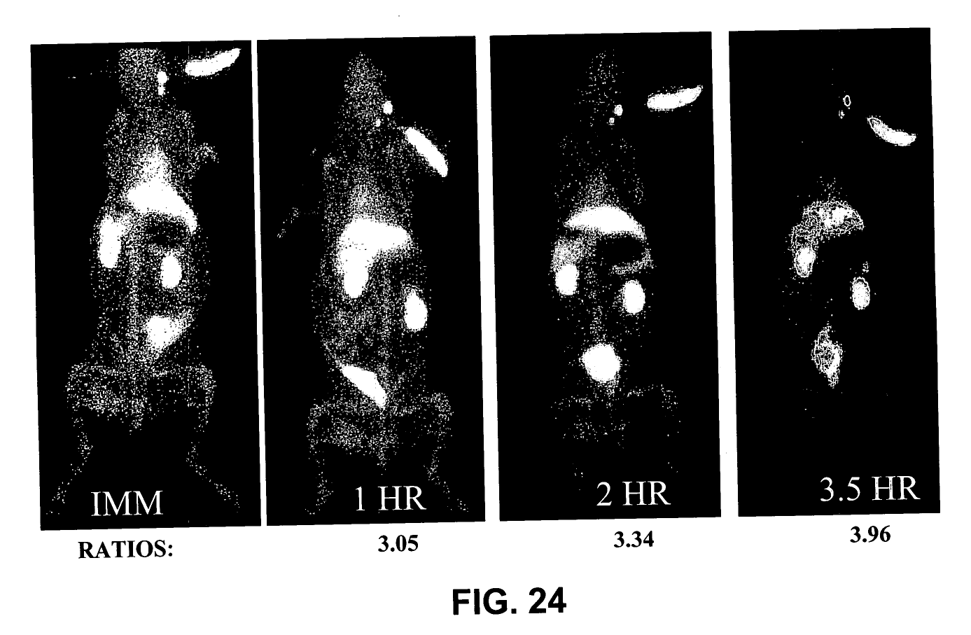

Dry Method: 100 mg of GAP (acid form, Mol Wt 750-3000) was put into 1.2 ml of anhydrous DMF and 1 ml of DMSO. 65.6 mg of DCC, 43.9 mg of DMAP and 101.2 mg of tetra-O-acetyl-β-D-mannopyranose (0.29 mmol) were added and stirred overnight at room temperature. 5 ml of NaHCO3 (1N) was added after removing solvent under reduced pressure. The mixture was filtered with 0.8 micrometer membrane filter and dialyzed with MW CO<500 membrane. 297.6 mg of white powder (GAP-DGAc salt form) after drying with lyophilizer was obtained. Structure, in vitro cellular uptake and in vivo biodistribution with different molecular weight GAP-DGAc (Tables 7-12) and imaging studies are shown in FIGS. 19-24.

TABLE 7
% ID/g comparison at 0.5, 2, 5, and 24 hrs.

| | Liver | Kidney | Tumor | Blood |
|---|---|---|---|---|
| G (5-20) | 3.4, 3.5, 2.9, — | 10, 13, 12, — | 0.5, 0.4, 0.3, — | 1.7, 0.9, 0.6, — |
| G-DG-6, | 7, 6, 6, 4 | 8, 10, 10, 6 | 0.4, 0.3, 0.1, 0.1 | 0.9, 0.4, 0.2, 0.1 |
| G-DG-10 | 4, 3.5, 3.5, 2 | 8, 12, 14, 8 | 0.4, 0.2, 0.2, 0.1 | 0.8, 0.3, 0.3, 0.1 |
| G-DG-20 | 3, 3, 3, 1.7 | 14, 19, 17, 11 | 0.4, 0.2, 0.2, 0.1 | 0.9, 0.3, 0.2, 0.1 |
| G-DG (5-20) | 6, 6, 5, 3 | 7, 10, 9, 6 | 0.4, 0.3, 0.2, 0.2 | 1.2, 0.5, 0.3, 0.1 |

TABLE 8
COUNT DENSITY COMPARISON

| | Tumor/Muscle | Tumor/Blood |
|---|---|---|
| G (5-20) | 4, 6, 6, — | 0.3, 0.4, 0.5, — |
| G-DG-6 | 4, 6, 8, 7 | 0.4, 0.6, 0.7, 0.9 |
| G-DG-10 | 4, 7, 9, 8 | 0.5, 0.6, 0.7, 1.2 |
| G-DG-20 | 3, 6, 7, 6 | 0.5, 0.7, 0.6, 0.9 |
| G-DG | 5, 8, 9, 13 | 0.3, 0.5, 0.8, 1.6 |
| (5-20) | | |

Note:
1. GAP (5-20, MW. 750-3,000) group has only three time point, 0.5, 2, and 4 hrs.
2. Blood background decreases when conjugated with sugar compared to GAP alone.
3. Higher molecular weight has less liver uptake. This may be due to more sugar moiety conjugated to the molecular. The conjugation yield determined by elemental analysis was 52% (w/w).
4. GAP-DGAC with 10 amino acid has the best value compared to 6, 10 and 20.

TABLE 9
Biodistribution of ^{99m}Tc-GAP in Breast Tumor-Bearing Rats
(MW. 750-3,000)
% of injected dose per gram of tissue weight (n = 3/time, interval, iv)

| | 30 MIN | 2 HOURS | 4 HOURS |
|---|---|---|---|
| BLOOD | 1.708 ± 0.050 | 0.924 ± 0.231 | 0.588 ± 0.019 |
| HEART | 0.473 ± 0.053 | 0.267 ± 0.057 | 0.180 ± .018 |
| LUNG | 0.852 ± 0.005 | 0.469 ± 0.114 | 0.333 ± 0.007 |
| LIVER | 3.454 ± 0.342 | 3.529 ± 0.333 | 2.876 ± 0.400 |
| SPLEEN | 1.628 ± 0.070 | 1.539 ± 0.390 | 1.072 ± 0.148 |
| KIDNEY | 10.141 ± 0.740 | 13.158 ± 4.090 | 11.702 ± 1.324 |
| INTESTINE | 0.285 ± 0.105 | 0.266 ± 0.041 | 0.151 ± 0.120 |
| UTERUS | 0.449 ± 0.016 | 0.388 ± 0.065 | 0.188 ± .038 |
| MUSCLE | 0.125 ± 0.016 | 0.066 ± 0.008 | 0.057 ± 0.025 |
| TUMOR | 0.520 ± 0.037 | 0.388 ± 0.038 | 0.323 ± .024 |
| THYROID | 0.653 ± 0.078 | 0.328 ± 0.111 | 0.339 ± 0.025 |
| STOMACH | 0.411 ± .032 | 0.301 ± 0.088 | 0.173 ± 0.017 |
| T/MUSCLE | 4.242 ± 0.881 | 5.981 ± 0.898 | 6.504 ± 2.908 |
| T/BLOOD | 0.304 ± 0.014 | 0.432 ± 0.082 | 0.549 ± 0.027 |
| UT/BLOOD | 0.263 ± 0.012 | 0.444 ± 0.154 | 0.321 ± 0.074 |
| UT/MUSCLE | 3.637 ± .466 | 6.009 ± 1.396 | 3.522 ± 0.802 |

Value shown represent the mean ± standard deviation of data from 3 animals

TABLE 10
Biodistribution of ^{99m}Tc-GAP-DGAC in Breast Tumor-Bearing Rats
(MW 750-3,000)
% of injected dose per gram of tissue weight (n = 3/time interval, iv)

| | 30 MIN | 2 HOURS | 5 HOURS | 24 HOURS |
|---|---|---|---|---|
| BLOOD | 1.173 ± 0.040 | 0.504 ± 0.008 | 0.273 ± 0.002 | 0.113 ± 0.012 |
| HEART | 0.238 ± 0.009 | 0.109 ± 0.005 | 0.08 ± 0.006 | 0.04 ± 0.001 |
| LUNG | 0.658 ± 0.025 | 0.303 ± 0.0045 | 0.185 ± 0.005 | 0.095 ± 0.006 |
| LIVER | 6.535 ± 0.413 | 5.56 ± 0.134 | 5.363 ± 0.470 | 3.348 ± 0.309 |
| SPLEEN | 1.634 ± 0.127 | 1.394 ± 0.196 | 1.049 ± 0.051 | 0.731 ± 0.040 |
| KIDNEY | 7.404 ± 0.155 | 9.541 ± 0.789 | 8.86 ± 0.317 | 6.41 ± 0.420 |
| INTESTINE | 0.231 ± 0.033 | 0.218 ± 0.043 | 0.18 ± 0.020 | 0.057 ± 0.007 |
| UTERUS | 0..416 ± 0.090 | 0.209 ± 0.022 | 0.138 ± 0.013 | 0.084 ± 0.001 |
| MUSCLE | 0.081 ± 0.007 | 0.032 ± 0.002 | 0.023 ± 0.001 | 0.014 ± 0.002 |
| TUMOR | 0.377 ± 0.009 | 0.267 ± 0.013 | 0.209 ± 0.006 | 0.177 ± 0.022 |
| THYROID | 0.494 ± 0.047 | 0.189 ± 0.017 | 0.14 ± 0.015 | 0.072 ± 0.006 |
| STOMACH | 0.393 ± 0.038 | 0.104 ± 0.004 | 0.09 ± 0.002 | 0.056 ± 0.008 |
| BRAIN | 0.0369 ± 0.037 | 0.018 ± 0.001 | 0.012 ± 0.006 | 0.007 ± 0.001 |
| T/MUSCLE | 4.685 ± 0.326 | 8.282 ± 0.399 | 9.067 ± 0.689 | 12.811 ± 1.486 |
| T/BLOOD | 0.321 ± 0.003 | 0.529 ± 0.019 | 0.766 ± 0.027 | 1.59 ± 0.210 |
| UT/BLOOD | 0.35 ± 0.063 | 0.414 ± 0.038 | 0.505 ± 0.047 | 0.76 ± 0.069 |
| UT/MUSCLE | 5.02 ± 720 | 6.448 ± 0.343 | 5.909 ± 0.263 | 6.145 ± 0591 |

Value shown represent the mean ± standard deviation of data from 3 animals

TABLE 11
Biodistribution of ^{99m}Tc-GAP⁶-DGAC in Breast Tumor-Bearing Rats
(MW 900)
% of injected dose per gram of tissue weight (n = 3/time interval, iv)

| | 30 MIN | 2 HOURS | 5 HOURS | 24 HOURS |
|---|---|---|---|---|
| BLOOD | 0.925 ± 0.117 | 0.375 ± 0.057 | 0.148 ± 0.092 | 0.089 ± 0.007 |
| HEART | 0.257 ± 0.030 | 0.099 ± 0.015 | 0.044 ± 0.027 | 0.030 ± 0.001 |
| LUNG | 0.599 ± 0.046 | 0.352 ± 0.054 | 0.125 ± 0.079 | 0.084 ± 0.004 |
| LIVER | 6.737 ± 0.425 | 5.813 ± 0.713 | 3.763 ± 2.319 | 3.844 ± 0.243 |
| SPLEEN | 1.523 ± 0.039 | 1.408 ± 0.149 | 0.918 ± 0.601 | 0.682 ± 0.095 |
| KIDNEY | 8.255 ± 0.702 | 9.604 ± 0.623 | 6.155 ± 3.806 | 6.731 ± 0.722 |
| INTESTINE | 0.220 ± 0.031 | 0.110 ± 0.025 | 0.076 ± 0.047 | 0.043 ± 0.003 |
| UTERUS | 0.343 ± 0.041 | 0.253 ± 0.045 | 0.070 ± 0.043 | 0.052 ± 0.001 |
| MUSCLE | 0.094 ± 0.014 | 0.036 ± 0.004 | 0.012 ± 0.008 | 0.012 ± 0.001 |
| TUMOR | 0.376 ± 0.046 | 0.225 ± 0.001 | 0.106 ± 0.067 | 0.083 ± 0.009 |
| THYROID | 0.342 ± 0.061 | 0.138 ± 0.019 | 0.082 ± 0.050 | 0.063 ± 0.004 |
| STOMACH | 0.201 ± 0.062 | 0.133 ± 0.021 | 0.051 ± 0.031 | 0.054 ± 0.004 |
| BRAIN | 0.030 ± 0.010 | 0.012 ± 0.002 | 0.007 ± 0.004 | 0.007 ± 0.001 |
| T/MUSCLE | 4.029 ± 0.744 | 6.347 ± 0.623 | 8.715 ± 0.166 | 7.033 ± 0.723 |
| T/BLOOD | 0.398 ± 0.036 | 0.635 ± 0.118 | 0.711 ± 0.051 | 0.930 ± 0.057 |
| UT/BLOOD | 0.373 ± 0.016 | 0.736 ± 0.218 | 0.477 ± 0.034 | 0.595 ± 0.058 |
| UT/MUSCLE | 3.682 ± 0.265 | 7.029 ± 1.245 | 5.932 ± 0.959 | 4.441 ± 0.258 |

Value shown represent the mean ± standard deviation of data from 3 animals

TABLE 12
Biodistribution of ^{99m}Tc-GAP¹⁰-DGAC in Breast Tumor-Bearing Rats
(MW. 1,500)
% of injected dose per gram of tissue weight (n = 3/time interval, iv)

| | 30 MIN | 2 HOURS | 5 HOURS | 24 HOURS |
|---|---|---|---|---|
| BLOOD | 0.851 ± 0.125 | 0.336 ± 0.017 | 0.285 ± 0.071 | 0.084 ± 0.010 |
| HEART | 0.224 ± 0.016 | 0.087 ± 0.009 | 0.071 ± 0.018 | 0.030 ± 0.002 |
| LUNG | 0.606 ± 0.122 | 0.267 ± 0.010 | 0.199 ± 0.048 | 0.077 ± 0.008 |
| LIVER | 4.085 ± 0.202 | 3.557 ± 0.274 | 3.571 ± 0.312 | 2.042 ± 0.316 |
| SPLEEN | 0.713 ± 0.259 | 0.819 ± 0.091 | 0.703 ± 0.079 | 0.301 ± 0.012 |
| KIDNEY | 8.604 ± 2.049 | 12.176 ± 1.204 | 14.424 ± 3.343 | 7.577 ± 0.473 |
| INTESTINE | 0.246 ± 0.015 | 0.183 ± 0.016 | 0.148 ± 0.028 | 0.049 ± 0.003 |
| UTERUS | 0.344 ± 0.014 | 0.161 ± 0.020 | 0.126 ± 0.031 | 0.056 ± 0.003 |
| MUSCLE | 0.096 ± 0.011 | 0.030 ± 0.003 | 0.023 ± 0.008 | 0.012 ± 0.001 |
| TUMOR | 0.395 ± 0.055 | 0.197 ± 0.029 | 0.188 ± 0.037 | 0.096 ± 0.005 |
| THYROID | 0.419 ± 0.050 | 0.132 ± 0.023 | 0.121 ± 0.034 | 0.066 ± 0.013 |
| STOMACH | 0.254 ± 0.016 | 0.097 ± 0.013 | 0.083 ± 0.016 | 0.038 ± 0.003 |
| BRAIN | 0.032 ± 0.003 | 0.013 ± 0.002 | 0.013 ± 0.003 | 0.005 ± 0.001 |
| T/MUSCLE | 4.117 ± 0.400 | 6.500 ± 0.446 | 8.868 ± 1.148 | 8.109 ± 0.188 |
| T/BLOOD | 0.466 ± 0.005 | 0.582 ± 0.059 | 0.675 ± 0.037 | 1.177 ± 0.149 |
| UT/BLOOD | 0.314 ± 0.158 | 0.474 ± 0.038 | 0.443 ± 0.004 | 0.690 ± 0.089 |
| UT/MUSCLE | 2.514 ± 1.293 | 5.308 ± 0.188 | 5.782 ± 0.487 | 4.821 ± 0.562 |
| BLOOD | 0.885 ± 0.095 | 0.318 ± 0.008 | 0.197 ± 0.012 | 0.073 ± 0.005 |
| HEART | 0.244 ± 0.021 | 0.095 ± 0.007 | 0.054 ± 0.001 | 0.029 ± 0.001 |
| LUNG | 0.491 ± 0.035 | 0.217 ± 0.004 | 0.140 ± 0.013 | 0.082 ± 0.015 |
| LIVER | 2.660 ± 0.497 | 3.061 ± 0.074 | 2.623 ± 0.199 | 1.754 ± 0.095 |
| SPLEEN | 0.763 ± 0.089 | 0.700 ± 0.028 | 0.594 ± 0.075 | 0.369 ± 0.016 |
| KIDNEY | 14.491 ± 0.956 | 19.395 ± 1.414 | 17.313 ± 0.069 | 11.480 ± 0.306 |
| INTESTINE | 0.364 ± 0.135 | 0.142 ± 0.022 | 0.098 ± 0.019 | 0.042 ± 0.001 |
| UTERUS | 0.686 ± 0.233 | 0.215 ± 0.043 | 0.119 ± 0.004 | 0.065 ± 0.007 |
| MUSCLE | 0.135 ± 0.012 | 0.038 ± 0.001 | 0.026 ± 0.000 | 0.016 ± 0.001 |
| TUMOR | 0.449 ± 0.026 | 0.231 ± 0.016 | 0.173 ± 0.006 | 0.085 ± 0.003 |
| THYROID | 0.425 ± 0.033 | 0.163 ± 0.009 | 0.112 ± 0.009 | 0.046 ± 0.007 |
| STOMACH | 0.310 ± 0.036 | 0.101 ± 0.003 | 0.075 ± 0.006 | 0.067 ± 0.021 |
| BRAIN | 0.029 ± 0.003 | 0.015 ± 0.001 | 0.013 ± 0.000 | 0.009 ± 0.002 |
| T/MUSCLE | 3.361 ± 0.190 | 6.008 ± 0.379 | 6.749 ± 0.154 | 5.571 ± 0.586 |
| T/BLOOD | 0.514 ± 0.030 | 0.725 ± 0.033 | 0.884 ± 0.055 | 1.181 ± 0.111 |
| UT/BLOOD | 0.738 ± 0.179 | 0.673 ± 0.124 | 0.606 ± 0.035 | 0.892 ± 0.067 |
| UT/MUSCLE | 4.862 ± 1.268 | 5.544 ± 0.938 | 4.630 ±± 0.112 | 4.171 ± 0.119 |

Value shown represent the mean ± standard deviation of data from 3 animals

Synthesis of GAP-LAS (di-supar)

Preparation of LAS-NH2: 100 mg of LAS-NHS (mono(lactosylamino)mono(succinimidyl)suberate) purchased from Pierce Chemical Company was dissolved into 0.6 ml of saline. 101.1 mg of ethylene diamine was added and stirred overnight at room temperature. The reaction mixture was filtered with 0.8 micrometer membrane filter and dialyzed with MW CO<500 membrane. 71.9 mg of white powder (LAS-NH2, Yield 79.3%) after drying with lyophilizer was obtained.

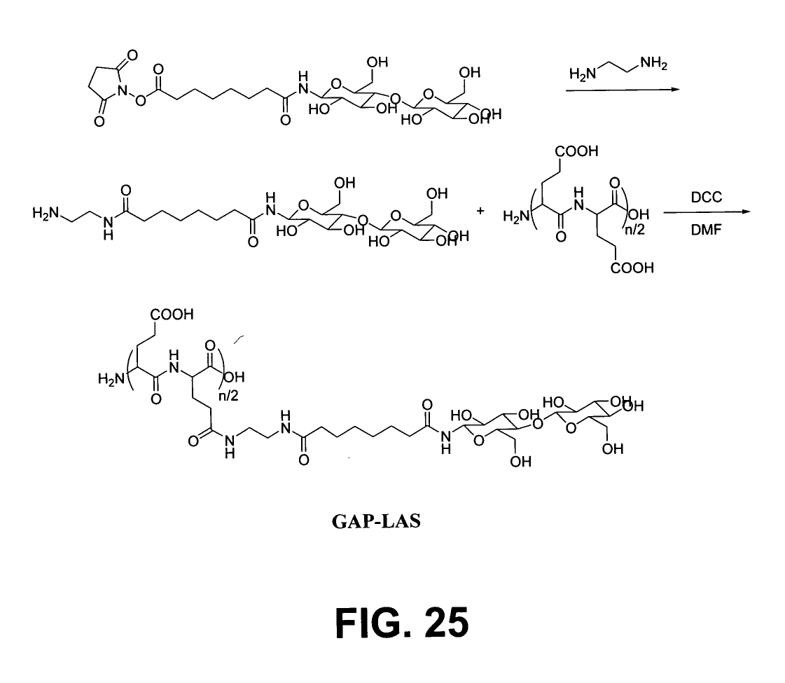

Synthesis of GAP-LAS: To a solution of GAP (Mol Wt 1500-3000) in 15 ml of anhydrous DMF, 23.1 mg of DCC and 50.3 mg of LAS-NH2 were added with stirring overmight at room temperature. Water layer was filtered with 0.8 micrometer membrane filter and dialyzed with MW CO<1000 membrane. 197.6 mg of white powder (GAP-LAS, yield 91.4%) after drying with lyophilizer was obtained. Synthetic scheme of LAS-NH2 and GAP-LAS are shown in FIG. 25. Proton NMR data confirmed the structures.

Synthesis of GAP-HCD (Polysaccharide)

Dry Method: 203 mg of GAP (acid form, Mol Wt 750-3000) was put into 7 ml of DMSO. 73.5 mg of DCC, 39.2 mg of DMAP and 419.8 mg of hydroxypropyl-p-cyclodextrin (0.29 mmol) were added and stirred overnight at room temperature. 5 ml of NaHCO3 (1N) was added after removing solvent under reduced pressure. The mixture was filtered with 0.8 micrometer membrane filter and dialyzed with MW CO<1,000 membrane. 197.1 mg of white powder (GAP-HCD salt form) after drying with lyophilizer was obtained.

In Vitro Cellular Uptake Studies

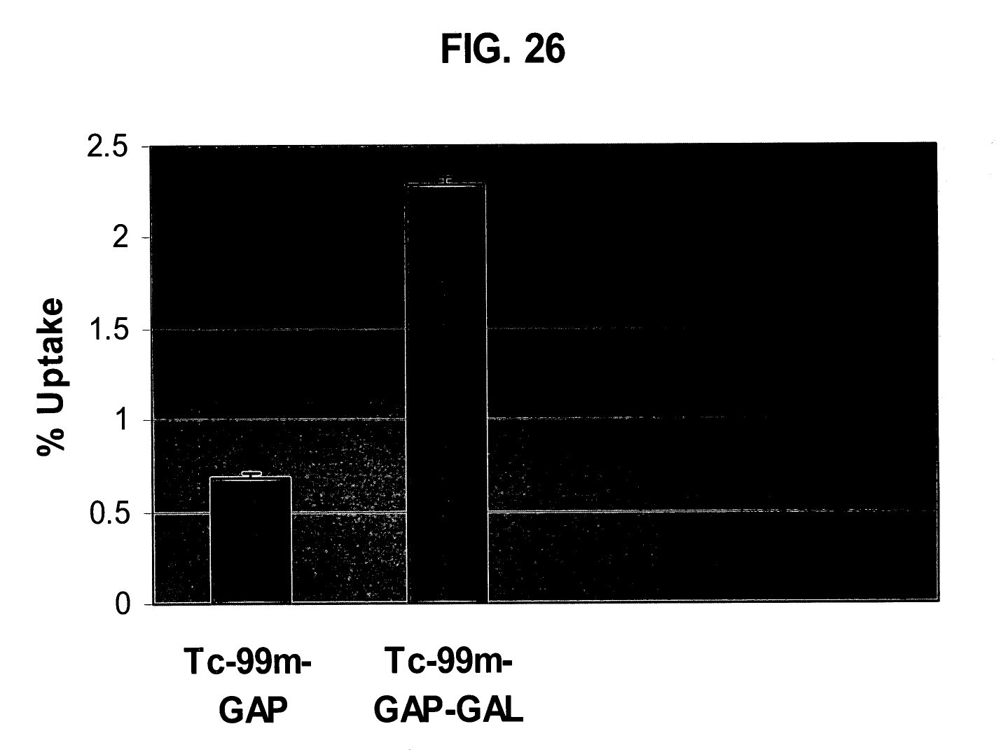

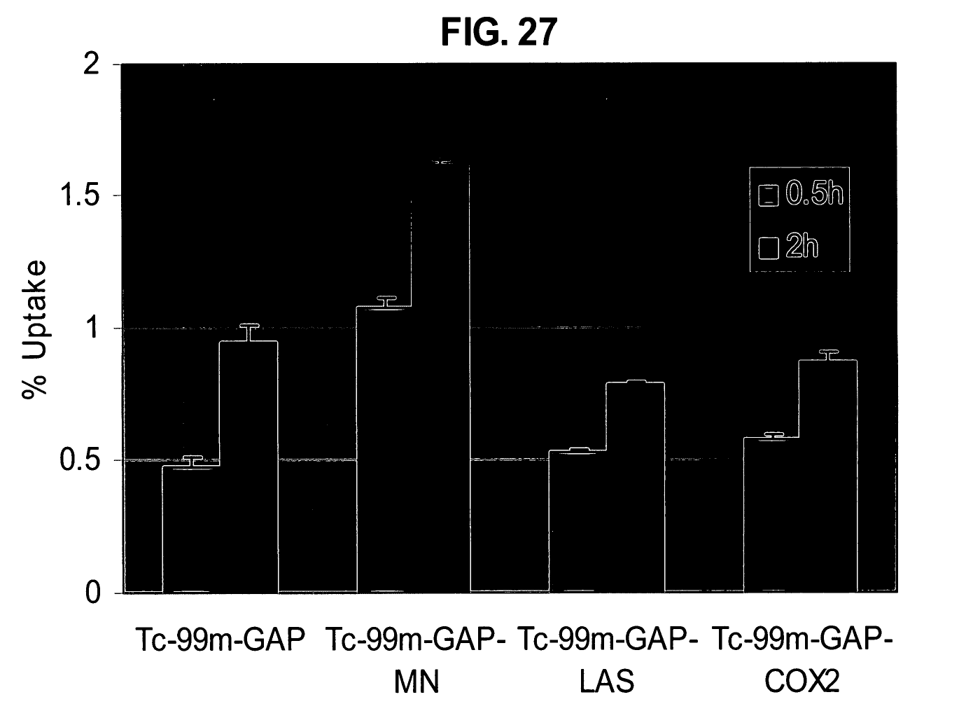

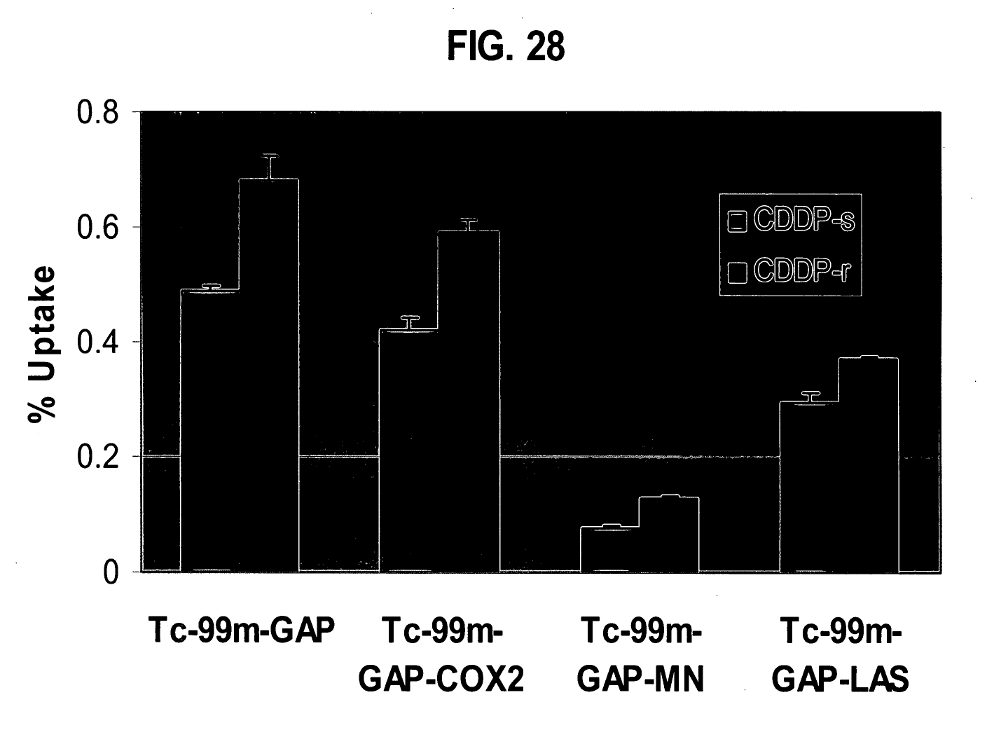

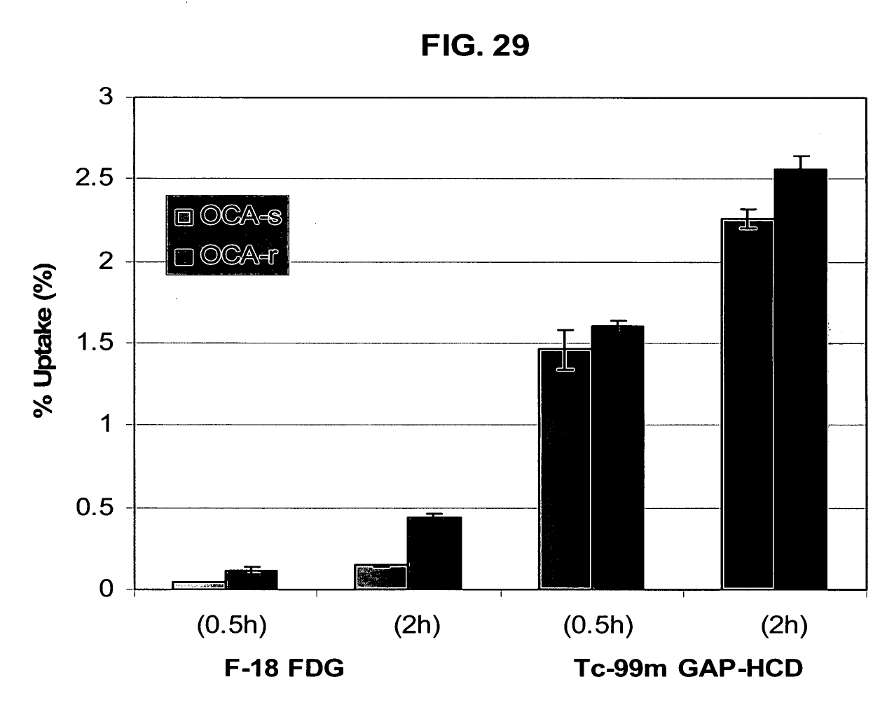

Two different cell lines were used for the assay. They were breast (13762NF) and ovarian (sensitive or resistant to cisplatin) cancer cell lines. Briefly, cells (50,000/well) were treated with 20 μl of ^{99m}Tc-GAP-sugar (GAL, LAS or HCD) or GAP (control) (100 μg/well, 1-21Ci/well). After 0.5-4 hrs incubation, the cells were washed twice with ice cold PBS (1 ml), and trypsin EDTA (0.1 ml) was added. After 2 min, PBS (0.4 ml) was added and the total volume containing cells was transferred to a test tube to count the activity. Each data represents an average of three measurements and that were calculated as percentage of uptake of ^{99m}Tc-GAP added. There was higher uptake in ^{99m}Tc-GAP-GAL group (FIG. 26), however, there was no marked difference of cellular uptake between ^{99m}Tc-labeled GAP and GAP-LAS (FIG. 27 and FIG. 28). GAP-HCD uptake was higher than FDG (FIG. 29).

Tissue Distribution Studies

Twelve female Fischer 344 rats (150±25 g) (n=3 rats/time point) were inoculated (i.m.) with mammary tumor cells derived from the RBA CRL-1747 cell line. The cells were cultured in Eagle's MEM with Earle's BSS (90%) and fetal bovine serum (10%). Tumor cells (10⁶ cells/rat) were injected (i.m.) into the hind legs. Studies were performed 14 to 17 days after implantation when tumors were approximately 1 cm in diameter.

In tissue distribution studies, each animal was injected (i.v., 10 μCi/rat, 10 μg/rat) with ^{99m}Tc-GAP-LAS or ^{99m}Tc-GAP. Rats were sacrificed at 0.5-4 hrs. The selected tissues were excised, weighed and counted for radioactivity by using a gamma counter. The biodistribution of tracer in each sample was calculated as percentage of the injected dose per gram of tissue wet weight (% ID/g).

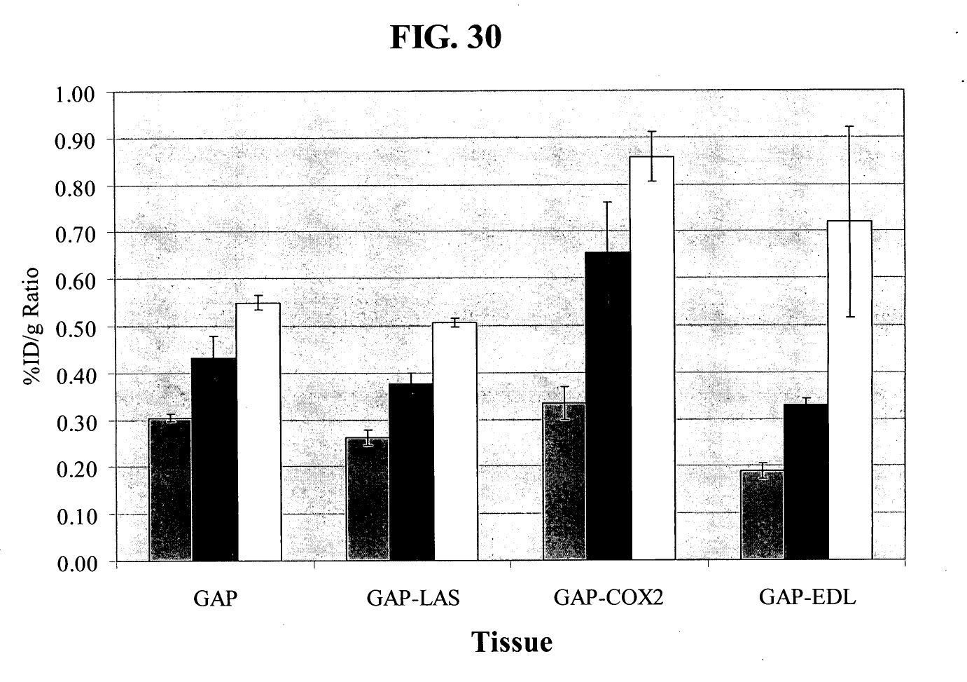

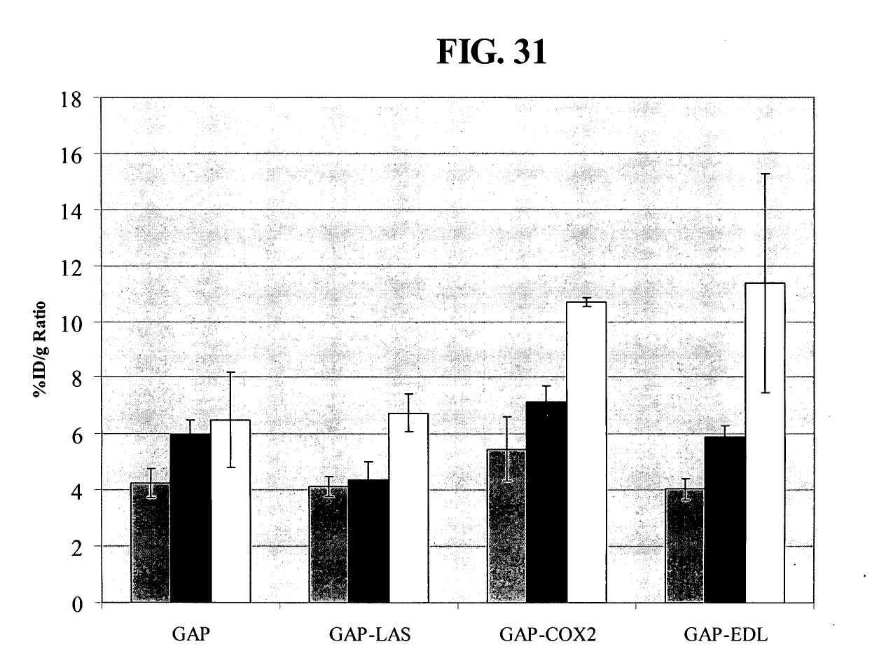

In vivo biodistribution studies showed that count density ratios for tumor-to-muscle (blood) in ^{99m}Tc-GAP-LAS groups were similar to ^{99m}Tc-GAP groups (Tables 3 and 13, FIG. 30 and FIG. 31). The finding suggest that further evaluation of different type of sugars.

TABLE 13
Biodistribution of ^{99m}Tc-GAP-LAS in Breast Tumor-Bearing Rats
% of injected dose per gram of tissue weight (n = 3/time, interval, iv)

| | 30 MIN | 2 HOURS | 4 HOURS |
|---|---|---|---|
| BLOOD | 1.375 ± 0.059 | 0.662 ± 0.050 | 0.385 ± 0.019 |
| HEART | 0.268 ± 0.020 | 0.152 ± 0.014 | 0.077 ± 0.001 |
| LUNG | 0.582 ± 0.032 | 0.300 ± 0.053 | 0.184 ± 0.006 |
| LIVER | 6.059 ± 0.695 | 4.062 ± 0.671 | 3.297 ± 0.208 |
| SPLEEN | 1.507 ± 0.151 | 1.590 ± 0.249 | 1.272 ± 0.157 |
| KIDNEY | 10.831 ± 0.902 | 12.859 ± 0.264 | 12.904 ± 1.863 |
| INTESTINE | 0.326 ± 0.016 | 0.467 ± 0.228 | 0.162 ± 0.021 |
| URINE | 11.091 ± 2.657 | 33.435 ± 5.320 | 16.242 ± 0.084 |
| MUSCLE | 0.089 ± 0.010 | 0.059 ± 0.011 | 0.029 ± 0.002 |
| TUMOR | 0.359 ± 0.010 | 0.250 ± 0.030 | 0.195 ± 0.006 |
| THYROID | 0.370 ± 0.058 | 0.222 ± 0.039 | 0.1134 ± 0.003 |
| STOMACH | 0.316 ± 0.019 | 0.194 ± 0.037 | 0.100 ± 0.007 |
| BRAIN | 0.042 ± 0.005 | 0.025 ± 0.000 | 0.016 ± 0.002 |
| T/MUSCLE | 4.131 ± 0.355 | 4.363 ± 0.632 | 6.750 ± 0.654 |
| T/BLOOD | 0.262 ± 0.016 | 0.376 ± 0.024 | 0.508 ± 0.009 |
| T/BRAIN | 8.676 ± 0.899 | 9.891 ± 1.278 | 13.012 ± 2.44 |
| T/LUNG | 0.621 ± 0.040 | 0.861 ± 0.063 | 1.068 ± 0.066 |
| HEART/MUS | 3.046 ± 0.123 | 2.690 ± 0.474 | 2.674 ± 0.204 |

Value shown represent the mean ± standard deviation of data from 3 animals

Scintigraphic Imaging and Autoradiographic Studies

Scintigraphic images were obtained at 0.5-4.0 hrs after i.v. injection of ⁹⁹“Tc-GAP-LAS or GAP-HCD (300 μCi/rat, iv) and imaged at 0.5-4.0 hrs in mammary tumor-bearing rats (derived from RBA CRL-1747 cell line). Whole-body autoradiogramrs were obtained by a quantitative image analyzer (Cyclone Storage Phosphor System, Packard, Meridian, Conn.). Following intravenous injection of ^{99m}Tc-GAP-GAL or GAP-LAS (100 μCi/rat, iv), athymic nude mice bearing human uterine sarcoma in the right leg were killed at 1 hour and the body was fixed in carboxymethyl cellulose (3%). The frozen body was mounted onto a cryostat (LKB 2250 cryomicrotome) and cut into 100 μm coronal sections. Each section was thawed and mounted on a slide. The slide was then placed in contact with multipurpose phosphor storage screen (MP, 7001480) and exposed for 16 hrs. Tumor could be well visualized at 0.54 hrs for both ^{99m}Tc-GAP-LAS or GAP-HCD groups. In autoradiograms, both mono- and di-sugars showed similar results.

### EXAMPLE 5

### ^{99m}Tc-GAP-Folate (FOL) for Folate Receptor Characterization

Synthesis of GAP-FOL

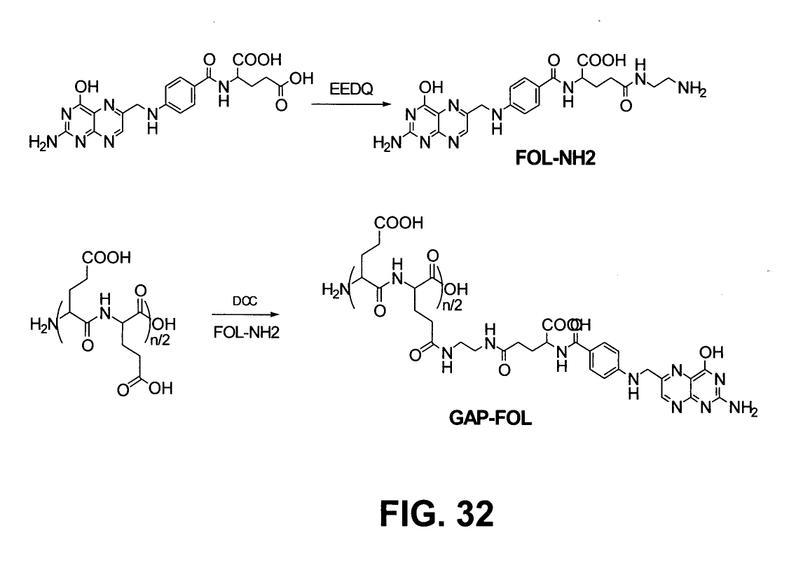

200 mg of GAP (Mol Wt 1500-3000) was dissolved in 10 ml of anhydrous DMF. 128 mg of DCC and 60 mg of FOL-NH2 (Ilgan et al., 1998a) were added and stirred overnight at room temperature. 15 ml of water was added after removing solvent under reduced pressure. Water layer was filtered with 0.8 micrometer membrane filter and dialyzed with MW CO<1,000 membrane. 207 mg of yellow powder (GAP-FOL, yield 80.3%) after drying with lyophilizer was obtained. The synthetic scheme is shown in FIG. 32. Proton NMR confirmed the structure.

### EXAMPLE 6

### ^{99m}Tc-GAP-Metronidazole (MN) for Hypoxia Characterization

Synthesis of GAP-MN

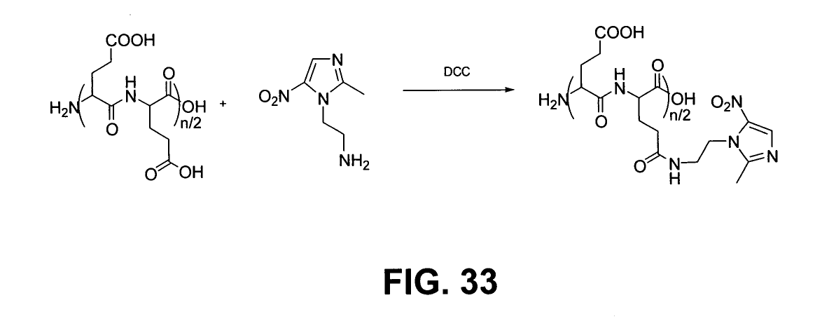

To a solution of 150 mg GAP (Mol Wt 1500-3000) in 10 ml anhydrous DMF and 70.4 mg DCC, 70.4 mg of MN (1-(2-aminoethyl)-2-methyl-5-nitroimidazole dihydrochloride monohydrate) was added and 1N-sodium hydroxide solution was added with stirring overnight at room temperature. Water layer was filtered with 0.8 micrometer membrane filter after removing solvent under reduced pressure and dialyzed with MW CO<1,000 membrane. 130.6 mg of brown powder (GAP-MN, Yield 64.5%) after drying with lyophilizer was obtained. The synthetic scheme is shown in FIG. 33. Proton NMR confirmed the structure.

Autoradiographic Studies

Whole-body autoradiograms were obtained by a quantitative image analyzer (Cyclone Storage Phosphor System, Packard, Meridian, Conn.). Following intravenous injection of ^{99m}Tc-GAP-MN or GAP (100 μCi/rat, iv), athymic nude mice bearing human uterine sarcoma in the right leg were killed at 1 hour and the body was fixed in carboxymethyl cellulose (3%). The frozen body was mounted onto a cryostat (LKB 2250 cryomicrotome) and cut into 100 μm coronal sections. Each section was thawed and mounted on a slide. The slide was then placed in contact with multipurpose phosphor storage screen (MP, 7001480) and exposed for 16 hrs. Tumor could be well visualized at 0.5-4 hrs for ^{99m}Tc-GAP-MN group.

### EXAMPLE 7

### ^{99m}Tc-GAP-Methotrexate (MTX) for Anti-Folate Characterization

Synthesis of GAP-MTX

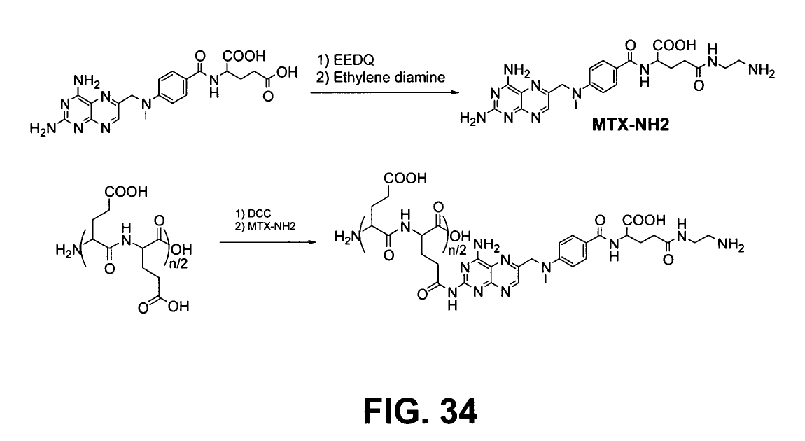

To a solution of 150 mg GAP (Mol Wt 1500-3000) in 10 ml of anhydrous DMF and 93 mg of DCC, 45 mg of MTXNH2 (Ilgan et al., 1998b) was added and stirred overnight at room temperature. 10 ml of water and 1 ml of 1N-sodium hydroxide solution were added after removing solvent under reduced pressure. Water layer was filtered with 0.8 micrometer membrane filter and dialyzed with MW CO<1,000 membrane. 139.8 mg of yellow powder (GAP-MTX, Yield 72.3%) after drying with lyophilizer was obtained. The synthetic scheme is shown in FIG. 34. The structure was confirmed by proton NMR.

In Vitro Cellular Uptake Studies

Breast (13762NF) cell line was used for the assay. Briefly, cells (50,000/well) were treated with 20 μl of ^{99m}Tc-GAP-MTX or GAP (control) (100 μg/well, 1-2 μCi/well). After 0.5-4 hrs incubation, the cells were washed twice with ice cold PBS (1 ml), and trypsin EDTA (0.1 ml) was added. After 2 min, PBS (0.4 ml) was added and the total volume containing cells was transferred to a test tube to count the activity. Each data represents an average of three measurements and that were calculated as percentage of uptake of ^{99m}Tc-GAP added. There was higher uptake in ^{99m}Tc-GAP-MTX than ^{99m}Tc-GAP group (FIG. 16).

### EXAMPLE 8

### ^{99m}Tc-GAP-Linolenate and Trimethyllysine for Lipid Metabolism Imaging

Preparation of Linolenic Acid-NH2

793.3 mg of ethylene diamine (13.2 mmol) was added into 1.2 ml of linolenic acid (4 mmol), DCC (1.65 g, 8 mmol), NHS (1.224 g, 8 mmol) and DMAP (0.325 g, 4 mmol) in 10 ml of chloroform. The mixture was stirred for 16 hours at room temperature. The solvent was then evaporated. The mixture was dissolved in 10 ml chloroform and washed twice with 7 ml Brine. The chloroform layer was dried over magnesium sulfate anhydrous, filtered and evaporated. The crude product weighed 2.57 g (crude yield 100%). The product was used for conjugation without further purification.

Synthesis of GAP-Linolenate

To a solution of 300 mg GAP (Mol Wt 1500-3000, 0.133 mmol) in 15 ml of anhydrous DMF, 275 mg of DCC (1.33 mmol) and 85 mg of linolenic acid-NH2 (0.133 mmol) were added. The mixture was stirred overmight at room temperature. The solvent was evaporated in vacuo. The resulting mixture was added NaOH (0.5 N) to adjust pH to 9-10. The solution was filtered with 0.8 micrometer membrane filter and dialyzed with MW CO<1000 membrane. 317.5 mg of white powder (GAP-linolenate) after drying with lyophilizer was obtained.

Synthesis of GAP-Trimethyllisine (TML)

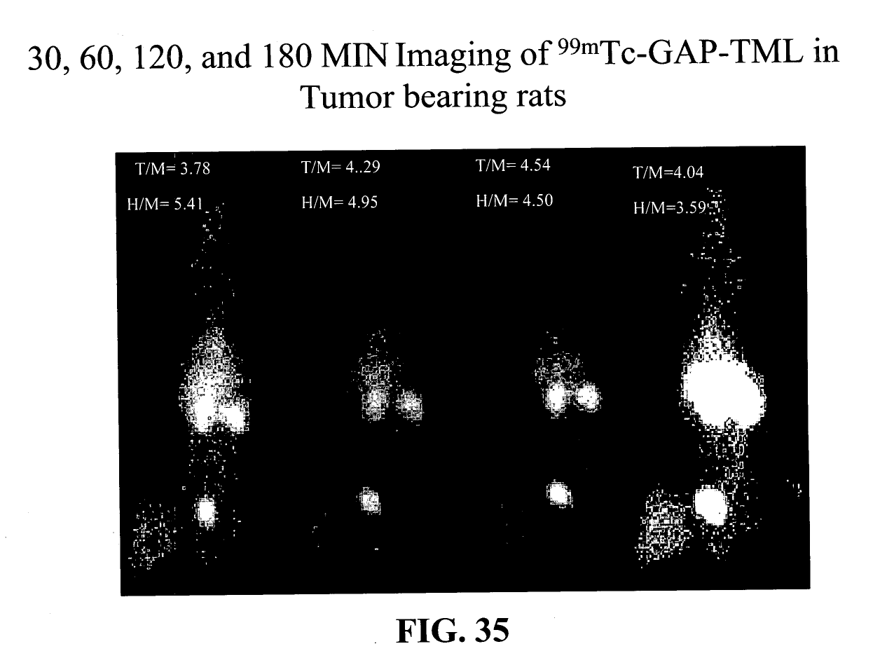

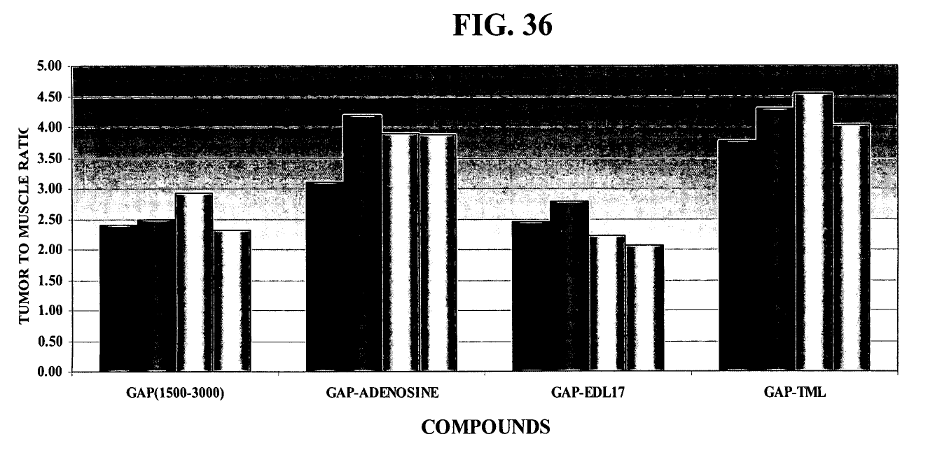

To a solution of 83 mg GAP (Mol Wt 2250, 0.036 mmol) in 5 ml of anhydrous DMF, 32.6 mg of DCC (0.158 mmol), 19.03 mg of DMAP (0.156 mmol) and 25 mg of TML (0.132 mmol) were added. The mixture was stirred overmight at room temperature. The solvent was evaporated in vacuo. The resulting mixture was added NaHCO3 (0.5 ml, 1 N) to adjust pH to 8. The solution was filtered with 0.8 micrometer membrane filter and dialyzed with MW CO<1000 membrane. 89.8 mg of white powder (GAP-TML, 84% yield) after drying with lyophilizer was obtained. Tumor could be well-visualized in rats (FIGS. 35-36).

### EXAMPLE 9

### ^{9m}Tc-GAP-Adenosine (ADN) for Proliferation Characterization

Synthesis of GAP-ADN

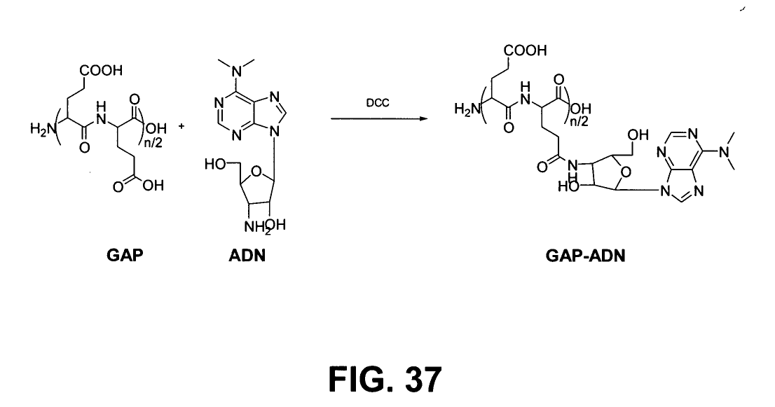

330.8 mg of GAP (Mol Wt 1500-3000) was dissolved in 15 ml of anhydrous DMF. 60.7 mg of DCC (dicyclohexylcarboimide) and 43.3 mg of 3′-amino-3′deoxy-N⁶,N⁶-dimethyladenosine (ADN) were added and the mixture was stirred overnight at room temperature. 10 ml of water was added after removing solvent under reduced pressure. The mixture was filtered with 0.8 micrometer membrane filter and dialyzed with MW CO<1,000 membrane. 290 mg of whiter powder (GAP-ADN, Yield 78.1%) was gained. The synthetic scheme is shown in FIG. 37. Proton NMR confirmed the structure. Tumor could be well-visualized in rats.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 6,692,724
U.S. Patent Appln. 09/599,152
U.S. Patent Appln. 10/627,763
U.S. Patent Appln. 10/672,142
U.S. Patent Appln. 10/703,405
U.S. Patent Appln. 10/732,919
Alauddin and Conti, Nucl. Med. Biol., 25(3):175-180 1998.
Alauddin et al., Nucl. Med. Biol., 23:787-792, 1996.
Alauddin et al., Nucl. Med. Biol., 26:371-376, 1999.
Alberico et al., Surg. Oncol. Clin. N. Am., 13(1):13-35, 2004.
Bird et al., Science, 242:423-426, 1988.
Bolhuis et al., Int. J. Cancer Suppl., 7:78-81, 1992.
Bush et al., Br. J. Cancer Suppl., 37(3):302-306, 1978.
Campbell et al., Cancer Res., 51(19):5329-5338 1991.
Canevari et al., Hybridoma., 12(5):501-507, 1993.
Chasselle et al, Lancet, 34B:143, 1995.
Chaudhary et al., Proc. Natl. Acad. Sci. USA, 87(3):1066-1070, 1990.
Chenu et al. Bone, 22(4):295-299, 1998.
Coney et al., Cancer Res., 54(9):2448-2455, 1994.
Dische, Int. J. Radiat. Oncol. Biol. Phys., 20(1):147-152, 1991.
Franklin et al., Int. J. Cancer Suppl., 8:89-95, 1994.
Gambhir et al., J. Nucl. Med., 39(11):2003-2011, 1998.
Gambhir et al., Proc. Natl. Acad. Sci. USA, 96(5):2333-2338, 1999.
Gambhir et al., Proc. Natl. Acad. Sci. USA, 97:2785-2790, 2000.
Gatenby et al., Int. J. Radiat. Oncol. Biol. Phys., 14(5):831-838, 1988.
Gelbard et al., J. Nucl. Med., 20(7):782-784, 1979.
Ginobbi et al., Anticancer Res., 17(1A):29-35, 1997.
Gray et al., Nature, 182(4640):952-953, 1958.
Hall et al., Radiat. Res., 114(3):415-424 1988.
Henson et al., AJNR Am. J. Neuroradiol., 25(6):969-972, 2004.
Holm, et al., APMIS, 102(11):828-836, 1994.
Hsuch and Dolnick, Biochem. Pharmacol., 45(12):2537-2545, 1993.
Huston et al., Biochemistry, 27(25):8945-8952, 1988.
Ilgan et al., Cancer Biother. Radiopharm., 13(3):177-184, 1998b.
Ilgan et al., Cancer Biother. Radiopharm., 13(6):427-435, 1998a.
Iyer et al., J. Nucl. Med., 42(1):96-105, 2001.
Koh et al., Int. J. Radiat. Oncol. Biol. Phys., 22:199-212, 1992.
Kranz et al., Proc. Natl. Acad. Sci USA, 92(20):9057-9061, 1995.
Layerman et al., Eur. J. Nucl. Med. Mol. Imaging, 29(5):681-690, 2002.
Leamon and Low, Biochem. J, 291 (Pt 3):855-860, 1993.
Leamon and Low, J. Biol. Chem., 267(35):24966-24971, 1992.
Leamon and Low, Proc. Natl. Acad. Sci. USA, 88(13):5572-5576 1991.
Lee and Low, J. Biol. Chem., 269(5):3198-3204, 1994.
Martin et al., J. Nucl. Med., 30:194-201, 1989.
Medical Letter Handbook of Adverse Drug Interactions, 199 Medical Letter, 34:78, 1992
Namavari et al., Nucl. Med. Biol., 27(2):157-162, 2000.
Nordsmark et al., Radiother. Oncol., 41(1):31-39, 1996.
Patrick et al., J. Neurooncol., 32(2):111-123, 1997.
Rasey et al., Int. J. Radiat. Oncol. Biol. Phys., 17(5):985-991 1989.
Rasey et al., Radiother. Oncol., 17(2):167-173, 1990.
Reed, Cancer Cell, 3:17-22, 2003.
Reiman et al., J. Nucl Med., 23(8):682-687, 1982.
Remington's Pharmaceutical Sciences, 18th Ed. Mack Printing Company, 1289-1329, 1990.
Ross et al., Cancer, 73(9):2432-2443, 1994.
Saha et al., Semin. Nucl. Med., 24(4):324-349,1994.
Senner et al., J. Natl. Cancer Inst., 88:140, 1994.
Sordillo et al., Am. J. Clin. Oncol., 5(3):285-289, 1982.
Strunk and Schild, Eur. Radiol., 14(6):1055-1062, 2004.
Tjuvajev et al., J. Nucl. Med., 43(8):1072-1083, 2002.
Vaalburg et al., Int. J. Rad. Appl Instrum. B, 19(2):227-237, 1992.
Valk et al., J. Nucl. Med., 33(12):2133-2137, 1992.
Warrell, Jr et al, N. Engl. J. Med., 329(3):177-189, 1993.
Weitman et al., Cancer Res., 52(12):3396-3401 1992b.
Weitman et al., Cancer Res., 52(23):6708-6711, 1992a.
Weitman et al., J Neurooncol., 21(2):107-112, 1994.
Westerhofet al., Cancer Res., 51(20):5507-5513, 1991.
Yaghoubi et al., J. Nucl. Med., 42:1225-1234, 2001.

What is claimed is:

1. A composition comprising:
a) a polypeptide comprising within its sequence two or more consecutive amino acids that will function to non-covalently bind valent metal ions; and
b) one or more valent metal ions non-covalently bound to at least one of the two consecutive amino acids.

2. The composition of claim 1, wherein the two or more consecutive amino acids are selected from the group consisting of aspartate, glutamate, an analog of aspartate, an analog of glutamate, cysteine, lysine, arginine, glutamine, asparagine, glycine, ornithine, and a non-naturally occuring amino acid that includes two more more carboxyl groups.

3. The composition of claim 1, wherein the two or more consecutive amino acids are glutamate residues.

4. The composition of claim 1, wherein the two or more consecutive amino acids are aspartate residues.

5. The composition of claim 1, wherein the polypeptide comprises at least 2 consecutive glutamate residues.

6. The composition of claim 5, wherein the polypeptide comprises at least 5 consecutive glutamate residues.

7. The composition of claim 6, wherein the polypeptide comprises at least 10 consecutive glutamate residues.

8. The composition of claim 7, wherein the polypeptide comprises at least 20 consecutive glutamate residues.

9. The composition of claim 8, wherein the polypeptide comprises at least 50 consecutive glutamate residues.

10. The composition of claim 1, wherein the polypeptide has a molecular weight of 300 to 30,000.

11. The composition of claim 10, wherein the polypeptide has a molecular weight of 750 to 9,000.

12. The composition of claim 1, wherein the polypeptide comprises at least 2 consecutive aspartate residues.

13. The composition of claim 12, wherein the polypeptide comprises at least 5 consecutive aspartate residues.

14. The composition of claim 13, wherein the polypeptide comprises at least 10 consecutive aspartate residues.

15. The composition of claim 14, wherein the polypeptide comprises at least 20 consecutive aspartate residues.

16. The composition of claim 15, wherein the polypeptide comprises at least 50 consecutive aspartate residues.

17. The composition of claim 1, wherein the polypeptide has a molecular weight of 300 to 30,000.

18. The composition of claim 17, wherein the polypeptide has a molecular weight of 750 to 9,000.

19. The composition of claim 1, wherein the polypeptide is capable of chelating three to five valent metal ions through coordination to carboxyl moieties of glutamate, aspartate, an analog of glutamate, or an analog of aspartate.

20. The composition of claim 1, wherein the valent metal ion is a radionuclide.

21. The composition of claim 1, wherein the valent metal ion is selected from the group consisting of Tc-99m, Cu-60, Cu-61, Cu-62, Cu-67, In-111, Tl-201, Ga-67, Ga-68, As-72, Re-186, Re-188, Ho-166, Y-90, Sm-153, Sr-89, Gd-157, Bi-212, Bi-213, Fe-56, Mn-55, Lu-177, a valent iron ion, a valent manganese ion, a valent cobalt ion, a valent platinum ion, and a valent rhodium ion.

22. The composition of claim 21, wherein the metal ion is Tc-99m.

23. The composition of claim 21, wherein the metal ion is Re-188.

24. The composition of claim 21, wherein the metal ion is Ga-68.

25. The composition of claim 1, further defined as comprising a second moiety bound to the polypeptide.

26. The composition of claim 25, wherein the second moiety is bound in an amide or ester linkage to a carboxyl moiety of the polypeptide.

27. The composition of claim 25, wherein the second moiety is a tissue targeting moiety, a diagnostic moiety, or a therapeutic moiety.

28. The compound of claim 27, wherein the tissue-targeting moiety is a targeting ligand.

29. The composition of claim 28, wherein the targeting ligand is a disease cell cycle targeting compound, an antimetabolite, a bioreductive agent, a signal transductive therapeutic agent, a cell cycle specific agent, a tumor angiogenesis targeting ligand, a tumor apoptosis targeting ligand, a disease receptor targeting ligand, a drug-based ligand, an antimicrobial, a tumor hypoxia targeting ligand, an agent that mimics glucose, amifostine, angiostatin, an EGF receptor ligand, monoclonal antibody C225, monoclonal antibody CD31, monoclonal antibody CD40, capecitabine, a COX-2 inhibitor, deoxycytidine, fullerene, herceptin, human serum albumin, lactose, leuteinizing hormone, pyridoxal, quinazoline, thalidomide, transferrin, or trimethyl lysine.

30. The composition of claim 29, wherein the the polypeptide comprises 5 to 60 consecutive glutamate residues, wherein the targeting ligand is estradiol, galactose, lactose, cyclodextrin, colchicin, methotrexate, paclitaxel, doxorubicin, celebrex, metronidazole, adenosine, penciclovir, carnetin, estradiol (position 3), estradiol (position 17), linolenic acid, glucosamine, tetraacetate mannose, or folate, and wherein the valent metal ion is 99 mTc.

31. The composition of claim 27, wherein the diagnostic moiety is an imaging moiety.

32. The composition of claim 31, wherein the imaging moiety is a contrast media.

33. The composition of claim 32, wherein the contrast media is selected from the group consisting of a CT contrast media, an MRI contrast media, an optical contrast media, and an ultrasound contrast media.

34. The composition of claim 33, wherein the contrast media is a CT contrast media.

35. The composition of claim 34, wherein the CT contrast media is selected from the group consisting of iothalamate, iohexol, diatrizoate, iopamidol, ethiodol, and iopanoate.

36. The composition of claim 33, wherein the contrast media is an MRI contrast media.

37. The composition of claim 36, wherein the MRI contrast media is selected from the group consisting of a gadolinium chelate, a manganese chelate, a chromium chelate, and iron particles.

38. The composition of claim 37, wherein MRI contrast media is Gd-DOTA, Mn-DPDP, or Cr-DEHIDA.

39. The composition of claim 33, wherein the contrast media is an optical contrast media.

40. The composition of claim 39, wherein the optical contrast media is selected from the group consisting of fluorescein, a fluorescein derivative, indocyanine green, Oregon green, a derivative of Oregon green derivative, rhodamine green, a derivative of rhodamine green, an eosin, an erythrosin, Texas red, a derivative of Texas red, malachite green, nanogold sulfosuccinimidyl ester, cascade blue, a coumarin derivative, a naphthalene, a pyridyloxazole derivative, cascade yellow dye, and dapoxyl dye.

41. The composition of claim 33, wherein the contrast media is an ultrasound contrast media is an ultrasound perfluorinated contrast media.

42. The composition of claim 41, wherein the ultrasound perfluorinated contrast media is selected from the group consisting of perfluorine or an analog of perfluorine.

43. The composition of claim 27, wherein the second moiety is a therapeutic moiety.

44. The composition of claim 43, wherein the therapeutic moiety is an anti-cancer moiety.

45. The composition of claim 44, wherein the anti-cancer moiety is selected from the group consisting of a chelator capable of chelating to a therapeutic radiometallic substance, methotrexate, epipodophyllotoxin, vincristine, docetaxel, paclitaxel, daunomycin, doxorubicin, mitoxantrone, topotecan, bleomycin, gemcitabine, fludarabine, and 5-FUDR.

46. The composition of claim 45, wherein the anti-cancer moiety is methotrexate.

47. The composition of claim 44, wherein the anti-cancer moiety is a therapeutic radiometallic substance selected from the group consisting of Re-188, Re-186, Ho-166, Y-90, Sr-89, Sm-153.

48. The composition of claim 44, wherein the anti-cancer moiety is a substance capable of chelating to a therapeutic metal selected from the group consisting of arsenic, cobolt, copper, selenium, thallium and platinum.

49. The composition of claim 1, wherein the valent metal ion that is non-covalently attached to the polypeptide can be imaged using PET or SPECT.

50. A composition comprising:
a) a polypeptide comprising within its sequence a tissue targeting amino acid sequence, a diagnostic amino acid sequence, and/or a therapeutic amino acid sequence; and
b) a valent metal ion attached to an amino acid residue of the polypeptide.

51. The composition of claim 50, wherein the polypeptide comprises two or more consecutive glutamate residues.

52. The composition of claim 50, wherein the polypeptide comprises two or more consecutive aspartate residues.

53. The composition of claim 50, wherein the polypeptide comprises 5 to 60 consecutive glutamate residues.

54. The composition of claim 50, wherein the polypeptide comprises 5 to 60 consecutive aspartate residues.

55. The composition of claim 50, wherein the valent metal ion is a radionuclide.

56. The composition of claim 50, wherein the valent metal ion is selected from the group consisting of Tc-99m, Cu-60, Cu-61, Cu-62, Cu-67, In-111, Tl-201, Ga-67, Ga-68, As-72, Re-186, Re-188, Ho-166, Y-90, Sm-153, Sr-89, Gd-157, Bi-212, Bi-213, a valent iron ion, a valent manganese ion, a valent cobalt ion, a valent platinum ion, or a valent rhodium ion.

57. The composition of claim 56, wherein the valent metal ion is Tc-99m.

58. The composition of claim 50, wherein the tissue-targeting amino acid sequence is a targeting ligand.

59. The composition of claim 58, wherein the targeting ligand is a disease cell cycle targeting compound, an antimetabolite, a bioreductive agent, a signal transductive therapeutic agent, a cell cycle specific agent, a tumor angiogenesis targeting ligand, a tumor apoptosis targeting ligand, a disease receptor targeting ligand, a drug-based ligand, an antimicrobial, a tumor hypoxia targeting ligand, an agent that mimics glucose, amifostine, angiostatin, an EGF receptor ligand, monoclonal antibody C225, monoclonal antibody CD31, monoclonal antibody CD40, capecitabine, a COX-2 inhibitor, deoxycytidine, fullerene, herceptin, human serum albumin, lactose, leuteinizing hormone, pyridoxal, quinazoline, thalidomide, transferrin, or trimethyl lysine.

60. The composition of claim 50, wherein the diagnostic amino acid sequence is an imaging amino acid sequence.

61. The composition of claim 60, wherein the imaging amino acid sequence is a contrast media selected from the group consisting of a CT contrast media, an MRI contrast media, and optical contrast media, and an ultrasound contrast media.

62. The composition of claim 50, wherein the therapeutic amino acid sequence is an anti-cancer amino acid sequence.

63. The composition of claim 62, wherein the anti-cancer amino acid sequence is capable of chelating to a therapeutic metal selected from the group consisting of arsenic, cobolt, copper, selenium, thallium and platinum.

64. A method of synthesizing an imaging agent, comprising:
a) obtaining a polypeptide comprising within its sequence two or more consecutive amino acids that will function to non-covalently bind valent metal ions; and
b) admixing said polypeptide with one or more valent metal ions and a reducing agent to obtain a valent metal ion-labeled polypeptide, wherein one or more valent metal ions non-covalently attaches to at least one of the two consecutive amino acids.

65. The method of claim 64, wherein the reducing agent is a dithionite ion, a stannous ion, or a ferrous ion.

66. The method of claim 64, wherein the polypeptide comprises 5 to 60 consecutive glutamate residues.

67. The method of claim 64, wherein the polypeptide comprises 5 to 60 consecutive aspartate residues.

68. The method of claim 64, wherein the polypeptide is capable of chelating three to five valent metal ions through coordination to carboxyl moieties of glutamate, aspartate, or the analog thereof.

69. The method of claim 64, wherein the valent metal ion is selected from the group consisting of Tc-99m, Cu-60, Cu-61, Cu-62, Cu-67, In-111, Tl-201, Ga-67, Ga-68, As-72, Re-186, Re-188, Ho-166, Y-90, Sm-153, Sr-89, Gd-157, Bi-212, Bi-213, a valent iron ion, a valent manganese ion, a valent cobalt ion, a valent platinum ion, or a valent rhodium ion.

70. The method of claim 69, wherein the metal ion is Tc-99m.

71. The method of claim 64, wherein the polypeptide is further defined as comprising a second moiety bound to the polypeptide.

72. The method of claim 71, wherein the second moiety is bound in an amide or ester linkage to a carboxyl moiety of the polypeptide.

73. The method of claim 71, wherein the second moiety is a tissue targeting moiety, a diagnostic moiety, or a therapeutic moiety.

74. The method of claim 73, wherein the tissue-targeting moiety is a targeting ligand.

75. The method of claim 74, wherein the targeting ligand is a disease cell cycle targeting compound, an antimetabolite, a bioreductive agent, a signal transductive therapeutic agent, a cell cycle specific agent, a tumor angiogenesis targeting ligand, a tumor apoptosis targeting ligand, a disease receptor targeting ligand, a drug-based ligand, an antimicrobial, a tumor hypoxia targeting ligand, an agent that mimics glucose, amifostine, angiostatin, an EGF receptor ligand, monoclonal antibody C225, monoclonal antibody CD31, monoclonal antibody CD40, capecitabine, a COX-2 inhibitor, deoxycytidine, fullerene, herceptin, human serum albumin, lactose, leuteinizing hormone, pyridoxal, quinazoline, thalidomide, transferrin, or trimethyl lysine.

76. The method of claim 64, further defined as a method of synthesizing an agent for imaging and chemotherapy.

77. The method of claim 64, further defined as a method of synthesizing an agent for dual imaging.

78. The method of claim 77, wherein the imaging is PET, SPECT, MRI, CT, or optical imaging.

79. A method of synthesizing an imaging agent, comprising:
a) obtaining a polypeptide comprising within its sequence a tissue targeting amino acid sequence, a diagnostic amino acid sequence, and/or a therapeutic amino acid sequence; and
b) admixing said polypeptide with one or more valent metal ions and a reducing agent to obtain a valent metal ion-labeled polypeptide.

80. The method of claim 79, wherein the reducing agent is a dithionite ion, a stannous ion, or a ferrous ion.

81. The method of claim 79, wherein the polypeptide comprises 5 to 60 consecutive glutamate residues.

82. The method of claim 79, wherein the polypeptide comprises 5 to 60 consecutive aspartate residues.

83. The method of claim 79, wherein the valent metal ion is selected from the group consisting of Tc-99m, Cu-60, Cu-61, Cu-62, Cu-67, In-111, Tl-201, Ga-67, Ga-68, As-72, Re-186, Re-188, Ho-166, Y-90, Sm-153, Sr-89, Gd-157, Bi-212, Bi-213, a valent iron ion, a valent manganese ion, a valent cobalt ion, a valent platinum ion, or a valent rhodium ion.

84. The method of claim 83, wherein the metal ion is Tc-99m.

85. The method of claim 79, wherein the tissue-targeting amino acid sequence is a targeting ligand.

86. The method of claim 85, wherein the targeting ligand is a disease cell cycle targeting compound, an antimetabolite, a bioreductive agent, a signal transductive therapeutic agent, a cell cycle specific agent, a tumor angiogenesis targeting ligand, a tumor apoptosis targeting ligand, a disease receptor targeting ligand, a drug-based ligand, an antimicrobial, a tumor hypoxia targeting ligand, an agent that mimics glucose, amifostine, angiostatin, an EGF receptor ligand, monoclonal antibody C225, monoclonal antibody CD31, monoclonal antibody CD40, capecitabine, a COX-2 inhibitor, deoxycytidine, fullerene, herceptin, human serum albumin, lactose, leuteinizing hormone, pyridoxal, quinazoline, thalidomide, transferrin, or trimethyl lysine.

87. The method of claim 79, wherein the diagnostic amino acid sequence is an imaging amino acid sequence.

88. The method of claim 87, wherein the imaging amino acid sequence is a contrast media selected from the group consisting of a CT contrast media, an MRI contrast media, and optical contrast media, and an ultrasound contrast media.

89. The method of claim 79, wherein the therapeutic amino acid sequence is an anti-cancer amino acid sequence.

90. The method of claim 89, wherein the anti-cancer amino acid sequence is capable of chelating to a therapeutic metal selected from the group consisting of arsenic, cobolt, copper, selenium, thallium and platinum.

91. A method of imaging a site within a subject comprising the steps of:
a) administering to the subject a diagnostically effective amount of a composition comprising a polypeptide comprising within its sequence two or more consecutive amino acids that will function to non-covalently bind valent metal ions wherein one or more valent metal ions is non-covalently bound to at least one of the two consecutive amino acids; and
b) detecting a signal from the valent metal ion-polypeptide chelate that is localized at the site.

92. The method of claim 91, wherein a signal is detecting using PET, CT, SPECT, MRI, optical imaging, or ultrasound.

93. The method of claim 91, further defined as a method of performing dual imaging and radiochemotherapy.

94. The method of claim 91, further defined as a method of performing dual imaging of a site within a subject.

95. The method of claim 91, wherein detecting a signal comprises using PET, SPECT, MRI, CT, optical imaging, or ultrasound.

96. A kit for preparing an imaging agent, said kit comprising a sealed container including a predetermined quantity of a polypeptide comprising within its sequence two or more consecutive amino acids that will function to non-covalently bind valent metal ions; and a sufficient amount of a reducing agent to non-covalently bind a valent metal ion to at least one of the two consecutive amino acids.

97. A kit for preparing an imaging agent, said kit comprising a sealed container including a predetermined quantity of a polypeptide comprising within its sequence a tissue targeting amino acid sequence, a diagnostic amino acid sequence, and/or a therapeutic amino acid sequence; and a sufficient amount of a reducing agent to attach one or more valent metal ions to the polypeptide.

98. The kit of claim 96 or 97, wherein the polypeptide comprises at least two consecutive glutamate or aspartate residues.

99. The kit of claim 98, wherein the polypeptide comprises at least five consecutive glutamate or aspartate residues.

100. A method of determining the effectiveness of a candidate substance as an imaging agent, said method comprising:
a) obtaining a candidate substance;
b) conjugating or chelating the candidate substance to a polypeptide comprising comprising within its sequence two or more consecutive amino acids that will function to non-covalently bind valent metal ions;
c) introducing the candidate substance-polypeptide conjugate to a subject; and
d) detecting a signal from the candidate substance-polypeptide conjugate to determine the effectiveness of the candidate substance as an imaging agent.


SEQUENCE LISTING (3 sequences)

SEQ ID NO: 1 - protein, 5 aa, Artificial Sequence
  Description of Artificial Sequence  Synthetic Peptide
GGGGS

SEQ ID NO: 2 - protein, 14 aa, Artificial Sequence
  Description of Artificial Sequence  Synthetic Peptide
EGKSSGSGSESKVD

SEQ ID NO: 3 - protein, 18 aa, Artificial Sequence
  Description of Artificial Sequence  Synthetic Peptide
KESGSVSSEQLAQFRSLD